SEQUENCE LISTING (35 sequences)

SEQ ID NO: 1 - DNA, 21 bp, Artificial Sequence
  nucleic acid encoding shRNA
gacggttagatgagcttgaga

SEQ ID NO: 2 - DNA, 21 bp, Artificial Sequence
  DNA encoding shRNA
tggtgttcaatcgcatactat

SEQ ID NO: 3 - DNA, 21 bp, Artificial Sequence
  DNA encoding shRNA
gctgaactgtctcctactact

SEQ ID NO: 4 - DNA, 21 bp, Artificial Sequence
  DNA encoding shRNA
gcgcgatagcgctaataattt

SEQ ID NO: 5 - DNA, 251 bp, Homo sapiens
tcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctcagccctccaggacaggctgcatcagaagaggccatcaagcaggtctgttcc

SEQ ID NO: 6 - DNA, 50 bp, Homo sapiens
gagatcagtgcggagctgtcaaagcgagcaggggtggcgccgggagtggg

SEQ ID NO: 7 - DNA, 50 bp, Homo sapiens
agcttaaatatccttccatcacctaatttacattcataaaattttaggtg

SEQ ID NO: 8 - DNA, 50 bp, Homo sapiens
gagataaatcggggcgaatttacagcccgggaggcacctgccgcgctaat

SEQ ID NO: 9 - DNA, 50 bp, Homo sapiens
gcttctgcctctttcacctctgtcctacttccggcgcgaaagcaggccac

SEQ ID NO: 10 - DNA, 50 bp, Homo sapiens
ttgcctgtctgagaatggctctgtccctgcttccgggtggtaggtggaca

SEQ ID NO: 11 - DNA, 601 bp, Rattus rattus
atccagctatcaatagaaactatgaaacagttccagggacaaagataccaggtccccaacaactgcaactttctgggaaatgaggtggaaaatgctcagccaaggaaaaagagggccttaccctctctgggacaatgattgtgctgtgaactgcttcatcaggccatctggccccttgttaataatctaattaccctaggtctaagtagagttgttgacgtccaatgagcgctttctgcagacttagcactaggcaagtgtttggaaattacagcttcagcccctctcgccatctgcctacctacccctcctagagcccttaatgggccaaacggcaaagtccagggggcagagaggaggtgctttggactataaagctagtggagacccagtaactcccaaccctaagtgaccagctacaatcatagaccatcagcaagcaggtatgtactctcctgggtgagcccggttcccccagccaaaactctagggactttaggaaggatgtgggttcctctcttacatggaccttttcctagcctcaaccctgcctatcttccaggtcattgttccaacatggccctgtggatgcgcttcctgc

SEQ ID NO: 12 - protein, 1368 aa, Streptococcus pyogenes [sequence omitted]

SEQ ID NO: 13 - RNA, 67 nt, Artificial Sequence
  tracrRNA
agcauagcaaguauaaauaaggcuaguccguuaucaacuugaaaaaguggcaccgagucggugcuuu

SEQ ID NO: 14 - DNA, 20 bp, Artificial Sequence
  crRNA
agcagtctcttcacaactgg

SEQ ID NO: 15 - DNA, 20 bp, Artificial Sequence
  crRNA
caccaaatacgatagatcag

SEQ ID NO: 16 - DNA, 20 bp, Artificial Sequence
  crRNA
tatcagagagttgagacacc

SEQ ID NO: 17 - DNA, 20 bp, Artificial Sequence
  crRNA
gagtgagtatagtcatccag

SEQ ID NO: 18 - DNA, 20 bp, Artificial Sequence
  crRNA
ttgaatcaaagtatggacac

SEQ ID NO: 19 - DNA, 20 bp, Artificial Sequence
  crRNA
aagccatctacagtgagtga

SEQ ID NO: 20 - DNA, 20 bp, Artificial Sequence
  crRNA
gcgcttatactacataggtg

SEQ ID NO: 21 - DNA, 249 bp, Artificial Sequence
  U6 promoter
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 22 - DNA, 378 bp, Artificial Sequence
  nucleic acid encoding U6 gRNA
ggcgcgccggatccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagagcgagctcttcggttttagagctagaaatagcaagttaaaataaggctagtccgttatcaacttgaaaaagtggcaccgagtcggtgctttttttggtaccggcgcgcc

SEQ ID NO: 23 - DNA, 83 bp, Artificial Sequence
  nucleic acid encoding tracrRNA
gttttagagctagaaatagcaagttaaaataaggctagtccgttatcaacttgaaaaagtggcaccgagtcggtgcttttttt

SEQ ID NO: 24 - DNA, 20 bp, Artificial Sequence
  targeted smad2 gene sequence
tcttcaggtttcacaccgga

SEQ ID NO: 25 - DNA, 20 bp, Artificial Sequence
  targeted smad2 gene sequence
ctaacccgaatgtgcaccat

SEQ ID NO: 26 - DNA, 20 bp, Artificial Sequence
  targeted smad2 gene sequence
gcatactatgaactaaacca

SEQ ID NO: 27 - DNA, 20 bp, Artificial Sequence
  scrambled control
gtgtagttcgaccattcgtg

SEQ ID NO: 28 - DNA, 22 bp, Artificial Sequence
  primer
ggctctccttcatgtcctctta

SEQ ID NO: 29 - DNA, 22 bp, Artificial Sequence
  primer
agcatattcgcagttttcgatt

SEQ ID NO: 30 - DNA, 22 bp, Artificial Sequence
  primer
aaccaagaatttgctgctcttc

SEQ ID NO: 31 - DNA, 22 bp, Artificial Sequence
  primer
gagaaagtcgaaacttgaacgg

SEQ ID NO: 32 - DNA, 22 bp, Artificial Sequence
  primer
ctgaccctctggtctgtcctat

SEQ ID NO: 33 - DNA, 22 bp, Artificial Sequence
  primer
ctgtgaagccgtctacagtgag

SEQ ID NO: 34 - DNA, 58 bp, Artificial Sequence
  AAV6-GFP-U6-m-SMAD2-shRNA
ccggtggtgttcaatcgcatactatctcgagatagtatgcgattgaacaccatttttg

SEQ ID NO: 35 - DNA, 21 bp, Artificial Sequence
  Targeting sequence
tggtgttcaatcgcatactat

(19) United States
(12) Patent Application Publication
(10) Pub. No.: US 2024/0191226 A1
(43) Pub. Date: Jun. 13, 2024

(54) SMAD2 INHIBITION IN BETA CELLS FOR TYPE 2 DIABETES THERAPY

(71) Applicant: University of Pittsburgh - Of the Commonwealth System of Higher Education, Pittsburgh, PA (US)

(72) Inventors: George Gittes, Gibsonia, PA (US); Krishna Prasadan, Pittsburgh, PA (US)

(73) Assignee: University of Pittsburgh - Of the Commonwealth System of Higher Education, Pittsburgh, PA (US)

(21) Appl. No.: 18/287,791

(22) PCT Filed: Apr. 20, 2022
(86) PCT No.: PCT/US2022/025594

§ 371 Date: Oct. 20, 2023

Related U.S. Application Data
(60) Provisional application No. 63/178,810, filed on Apr. 23, 2021.

(51) Int. Cl. C12N 15/11; A61K 31/16; A61K 31/7088; A61K 38/46; A61P 3/10; C12N 9/22; C12N 15/113; C12N 15/86; C12N 15/90

(52) U.S. Cl. CPC C12N 15/11; A61K 31/16; A61K 31/7088; A61K 38/465; A61P 3/10; C12N 9/22; C12N 15/113; C12N 15/86; C12N 15/907; C12N 2310/20; C12N 2310/531; C12N 2750/14143; C12N 2800/80

(57) ABSTRACT

Methods are disclosed herein for treating a subject with type 2 diabetes. These methods include administering to the subject a therapeutically effective amount of an inhibitor that decreases expression of smad2. In some embodiments, the inhibitor is an inhibitory RNA, a small molecule, or a CRISPR-Cas9 system.

### CROSS REFERENCE TO RELATED APPLICATIONS

This claims the benefit of U.S. Provisional Application No. 63/178,810, filed Apr. 23, 2021, which is incorporated by reference herein.

### STATEMENT OF GOVERNMENT SUPPORT

This invention was made with Government support under project number DK111460 from the National Institutes of Health. The United States Government has certain rights in the invention.

### FIELD OF THE DISCLOSURE

This relates to the field of diabetes, specifically to the use of inhibitor of Mothers against decapentaplegic homolog (SMAD)2 for the treatment of type 2 diabetes (T2DM).

### BACKGROUND

Diabetes is a significant health problem in the United States and worldwide. According to the CDC National Diabetes Statistics Report (2017), in 2015 the prevalence of diabetes in the USA was 30.3 million (9.4%), and 84.1 million Americans aged 18 and older had prediabetes. Diabetes remains the 7th leading cause of death in the USA (Mayer-Davis et al., N Engl J Med. 2017; 376(15):1419-29). With the worsening obesity epidemic, the incidence of T2DM has been rising (Mayer-Davis et al., N Engl J Med. 2017; 376(15):1419-29). Obesity is an insulin-resistant state that places significant stress on the pancreatic β-cells as they augment insulin secretion to overcome the insulin resistance. As long as the β-cells can increase their insulin secretion sufficiently to overcome the insulin resistance, glucose tolerance remains normal (Gastaldeli et al., Diabetologia. 2004; 47(1):31-9). With long-standing insulin resistance, there can be early β-cell dysfunction (Abdul-Ghani et al., Am J Physiol Endocrinol Metab. 2008; 295(2):E401-6) and progressive β-cell loss leading to the onset of overt diabetes (Hanley et al., Endocrinology. 2010; 151(4):1462-72). Studying β-cell biology and signaling pathways that regulate insulin secretion, proliferation and adaptive capacity of β-cells, toward the goal of understanding the pathogenesis of T2DM, can lead to new therapeutic strategies.

### SUMMARY OF THE DISCLOSURE

Methods are disclosed herein for treating a subject with T2DM. These methods include administering to the subject a therapeutically effective amount of an inhibitor that decreases expression of smad2.

The foregoing and other features and advantages of the invention will become more apparent from the following detailed description of several embodiments which proceeds with reference to the accompanying figures.

### BRIEF DESCRIPTION OF THE FIGURES

FIGS. 1A-1D. Generation of β-cell-specific smad2 knockout mice. (A) Schematic for ins1^{cre} mediated deletion of smad2. Smad2^{fx/fx} mice possess loxP sites flanking exon2 of the genomic smad2. By crossing smad2^{fx/fx} mice with ins1^{cre} transgenic mice, β-cell-specific smad2 null mutant (smad2-βKO) mice were generated. (B) Smad2 protein expression was analyzed in isolated islets of 14-week-old smad2^{fx/fx} (n=3) and smad2-βKO mice (n=4). Cropped gels are displayed (left panel). Western blot results were analyzed by densitometry (right panel), showing decreased smad2 protein expression in smad2-βKO (squares). R-Actin was used as a protein loading control for Western analysis. (C) Representative pancreatic tissue sections from 14-week-old smad2^{fx/fx} and smad2-βKO mice (n=3 per group) were immunostained for p-smad2 (Ser-465/467), showing decreased detectable p-smad2 in smad2-βKO mice. Scale bar is 50 μm. (D) Smad2 mRNA by real-time PCR in pancreatic islets of 14-week-old smad2^{fx/fx} (circles) and smad2-βKO (squares) mice; n=3 per group. Values were normalized against the housekeeping gene (Pipia). Data are represented as mean±SD, ** p<0.01, ***p<0.001.

FIGS. 2A-2G. Improved glucose tolerance and GSIS in smad2-βKO mice and isolated islets. (A) IPGTT was done for 14-week-old mice, showing improved glucose tolerance in smad2-βKO mice (squares) compared to their littermate controls (circles); n=7 per group. (B) Analysis of area under the curve (AUC) for the IPGTT (A). (C) In vivo GSIS done for 14-week-old controls (circles) and smad2-βKO (squares) mice; n=4 per group. Smad2-βKO exhibited increased serum insulin levels at 15 and 30 min. (D) IPITT was done for 14-weeks-old did not show significant difference between smad2-βKO mice (squares) and their littermate controls (circles); n=4 per group. (E) ex vivo static GSIS on isolated islets from 14-week-old mice. Islets isolated from smad2-βKO mice (squares) showed higher insulin release in response to high glucose concentration (20 mM) compared to their controls (circles); n=3 per group. Data are represented as mean±SD, *p<0.05, **p<0.01, ***p<0.001, ns=no significance. (F) ex vivo islet perifusion assay with islets harvested from 14-week-old control (circles, n=3) and smad2-βKO mice (squares, n=4). Islets from smad2-βKO showed higher insulin release in response to high glucose concentration (20 mM) and KCl perifusion. The differences between the two groups in each of the three conditions (low glucose, high glucose, and KCl) were analyzed by AUC followed by unpaired Student t-test. Only significant p-values are depicted. (G) Inulin content was compared between islets isolated from 14-week-old smad2 KO mice (squares, n=4) and their littermate controls (circles, n=5). The insulin content was higher in smad2-βKO islets. Data are represented as mean insulin concentration±SD, ***p<0.001.

FIGS. 3A-3E. Loss of smad2 in β-cells enhances expression of genes involved in β-cell function and increases β-cell mass and proliferation. (A) Expression levels of insulin 2 gene, certain β-cell differentiation markers, ATP-sensitive potassium channel subunits (Abcc8, Kcnj11), voltage-gated calcium channel subunits alpha1C and alpha1D (Cacna1c, Cacna1d), Ca²⁺-channel and insulin secretion specific mRNAs; Synaptotagmin-2, -7 and-9 (Syt2, Syt7, Syt9), Piccolo (Pclo) and PICK1(Pick1) were all quantified by RT-PCR in cells from isolated islets of 14-week old smad2^{fx/fx} (white bars, n=3) and smad2-βKO mice (red bars, n=3). Values were normalized against the housekeeping gene (Pipia), with the latter being consistent across all conditions. Data are represented as mean±SD, * p<0.05, **p<0.01. (B) Confocal images of dispersed islet-cells labeled with Fluo-4 (left panel). Mean Fluo-4 intensity were analyzed (right panel) showed that smad2-βKO mice had increased intensity of fluo-4 after high glucose concentration (20 mM) and KCl stimulation compared to their littermate controls, the differences between the two groups were analyzed by AUC followed by unpaired Student t-test, with p<0.001. (C) Calculation of β-cell mass in 14-week-old smad2-βKO mice (squares, n=4) and their littermate controls (circles, n=3), showing a significant increase in β-cell mass in smad2-βKO mice compared to their littermate controls. (D) Representative images and quantification of co-immunostaining for insulin and BrdU in 14-week-old smad2-βKO mice (squares), and their littermate controls (circles); n=4 per group, islets from smad2-βKO mice had higher BrdU⁺/Ins⁺ cells compared to control mice. (E) Partial pancreatectomy (PPX) or sham surgery were done on 14-week-old smad2-βKO mice (squares, n=5 in PPX group and n=4 in sham group) and their littermate controls (circles, n=4 in PPX group and n=3 in sham group). One week after surgery β-cell proliferation was determined by BrdU incorporation assay. Representative images and quantification showed that smad2-βKO islets had higher BrdU⁺/Ins⁺ cells compared to their littermate controls. All data are represented as mean±SD, * p<0.05, *** p<0.001. Scale bar is 100 um.

FIGS. 4A-4H. Loss of smad2 in β-cells improves HFD-induced dysglycemia and improves GSIS in vivo and in isolated islets. (A) Body weight of 18-week-old control smad2-βKO (squares) and their littermate controls (circles) on regular chow (REG) or 60% high fat diet (HFD); n=7 per group. (B) After 12 weeks of HFD feeding, IPGTT was done for 18-week-old mice showing, improved glucose tolerance in smad2-βKO mice (squares, n=9) compared to their littermate controls (circles, n=8). (C) Analysis of AUC for the IPGTT. (D) In vivo GSIS was done, after 12 weeks of HFD, for smad2-βKO mice (squares), and littermate controls (circles); n=6 per group. Compared to controls, smad2-βKO mice exhibited increased serum insulin levels at 15 min. (E) After 12 weeks of HFD feeding, HbA1c was significantly lower in smad2-βKO mice (squares) compared to their littermate controls (circles); n=5 per group. (F) ex vivo GSIS on isolated islets from 18-week-old HFD-fed mice. Islets isolated from smad2-βKO mice (squares) showed higher insulin release in response to high glucose concentration (20 mM) compared to their controls (circles); n=4 per group. All data are represented as mean±SD, ns=no significance, *p<0.05, ** p <0.01, *** p<0.001. (G) ex vivo islet perifusion assay with islets harvested from 18-week-old smad2-βKO mice (squares) and their littermate controls (circles), after 12 weeks of HFD feeding. Islets from smad2-βKO showed higher insulin release in response to high glucose concentration (20 mM). The differences between the two groups in each of the three conditions (low glucose, high glucose, and KCl) were analyzed by AUC followed by unpaired Student t-test. Only significant p-values are depicted. (H) After 12 weeks of HFD, inulin content was compared between islets isolated from 18-week-old smad2 KO mice (squares) and their control littermates (circles); n=4 per group. There was no significant difference between the two groups. Data are represented as mean insulin concentration±SD, ns=no significance.

FIGS. 5A-5D. Loss of smad2 improves insulin sensitivity markers in the liver and peripheral tissues in HFD-mice. (A) IPITT was done after 12 weeks of HFD feeding showed significant improvement in insulin sensitivity in HFD-smad2-βKO mice (squares, n=9) compared to their littermate HFD-controls (circles, n=6). Data present percent change in blood glucose. (B) HOMA-IR. Data are represented as mean±SD, *p<0.05 **p<0.01. (C) Immunoblot analysis of p-Akt (Ser473) and total Akt in the liver, skeletal muscle and adipose tissue of smad2-βKO mice and littermate controls, after 12 weeks HFD. Cropped gels are displayed (left panel). Western blot results were analyzed by densitometry. The bar chart (right panel) represent quantitative comparisons between the two groups (circles=controls, and squares=smad2-βKO). Smad2-βKO had higher expression of p-Akt in liver, muscle and adipose tissue compared to their littermate controls. β-actin was used as a loading and transfer control. The relative density is shown as the mean±SD; n=3 per group, *p<0.05. (D) Representative images (left panel) and quantification for Oil-Red-O staining of liver specimens (right panel) showed significantly lower hepatic fat in smad2-βKO compared to their littermate controls after 12 weeks of HFD (n=4 per group). Data are shown as mean±SD. ***p<0.001. Scale bar is 100 μm.

FIGS. 6A-6D. Loss of smad2 in β-cells decreases ER stress, increases β-cell proliferation and mass in HFD-fed mice. (A) Calculation of R-cell mass in smad2-βKO mice (squares, n=4) and littermate controls (circles, n=3), after 12 weeks HFD, showing a significant increase in R-cell mass in smad2-βKO mice. (B) Representative images and quantification of co-immunostaining for insulin and BrdU in smad2-βKO mice (squares) and littermate controls (circles), after 12 weeks HFD; n=4 per group. Smad2-βKO islets had higher BrdU⁺/Ins⁺ cells compared to control mice. (C) Expression levels of certain ER stress markers (Bip, Ddit3 and Atf4) were quantified by RT-PCR in islets isolated from 18-week-old control mice on regular diet (diamonds, n=3), HFD-control (circles, n=4), and HFD-smad2-βKO mice (squares, n=4), after 12 weeks of HFD. Islets isolated from HFD-smad2-βKO mice showed a significant lower expression of ER-stress markers compared to HFD-controls. Values were normalized against the housekeeping gene (Pipia), with the latter being consistent across all conditions. (D) Islets isolated from 18-week-old control mice on regular diet or HFD, and HFD-smad2-βKO mice were analyzed for the phosphorylation of URP proteins, p-PERK (Thr980) and p-elF2α (Ser51). Cropped gels are displayed (left panel). Densitometry data (right panel) are the phosphorylated protein levels normalized to the housekeeping gene, β-Actin, and relative to its expression in regular diet controls. diamonds=regular diet controls, circles=HFD-controls, and squares=HFD-smad2-βKO; n=3 per group. Data are represented as mean±SD, * p<0.05, **p<0.01, *** p<0.001, ns=no significance.

FIGS. 7A-7D. Loss of smad2 in β-cells is associated with enhanced expression of certain β-cell markers. Representative images (right panels) with quantification (left panels) of co-immunostaining for insulin with MafA (A), Pdx1 (B), Nkx6.1 (C) and NeuroD1 (D) in 14-week-old control (n=3) and smad2-βKO (n=4), showing a higher expression of MafA, Pdx1, and NeuroD1 in smad2-βKO compared to their littermate controls. *p<0.05 **p<0.01, ***p<0.001, ns=no significance. Data are represented as mean±SD. Scale bar is 100 μm.

FIGS. 8A-8B (A) Expression levels of certain ER-stress markers (Bip, Ddit3 and Atf4) were quantified by RT-PCR in islets isolated from 18-week-old control mice (circles, n=3) or smad2-βKO mice (squares, n=4) on regular diet, showing a significant lower expression of ER-stress markers in smad2-βKO islets compared to controls. Values were normalized against the housekeeping gene (Pipia), with the latter being consistent across all conditions. (B) Islets isolated from control mice (n=3) or smad2-βKO mice (n=4) on regular diet, were analyzed for the phosphorylation of URP proteins, p-PERK (Thr980) and p-elF2α (Ser51). Cropped gels are displayed (left panel). Densitometry data (right panel) are the phosphorylated protein levels normalized to the house keeping gene, R-Actin and relative to its expression in controls. There was no significant difference between the 2 groups. Data are represented as mean±SD. *p<0.05 **p<0.01, ns=no significance.

FIG. 9. Intraperitoneal glucose tolerance test (IPGTT) and area under the curve one week before tamoxifen treatment show impaired glucose tolerance in mice containing a cre inducible smad2-βKO insertion that are fed on HFD for 12 weeks.

FIGS. 10A-10F. After tamoxifen-mediated deletion of smad2 expression in beta cells, intraperitoneal glucose tolerance test (IPGTT) and area under the curve at one week (A, B) and three weeks (C, D) and nine weeks (E, F) showed improved glucose tolerance in mice fed HFD for 12 weeks and continued on HFD.

FIGS. 11A-11F. Fifteen weeks after the first dose of tamoxifen-mediated deletion of smad2 expression in beta cells, the mice still on HFD failed the intraperitoneal glucose tolerance test (IPGTT) (A, B). Following repeated IPGTT failures, 22 weeks after the first tamoxifen injection, the mice received a second regimen of tamoxifen. One week after the second dose, loss of beta-cell specific smad2 deletion improved glucose tolerance (C, D) again in mice still on HFD for twenty-three weeks. After the second dose of tamoxifen, the mice maintained on HFD for 29 weeks showed improved glucose tolerance (E, F).

FIGS. 12A-12C. IPGTT shows substantial improvement in glucose clearance following smad2 knockout in adult beta cells (A). In vivo GSIS show improvement in insulin secretion (B). Homeostatic Model Assessment for Insulin Resistance (HOMA-IR) show improved insulin resistance in smad2 knockout mice, similar to littermate mice on regular diet and received Oil treatment instead of tamoxifen (C).

FIGS. 13A-13C. Within three weeks after deleting smad2 expression peripheral insulin sensitivity improved in the knockout mice. ITT performed one week prior to smad2 knockout show increased insulin resistance in mice fed on HFD for 12 weeks, compared to regular CHOW diet (A). ITT showed improved insulin resistance in smad2 knockout mice three weeks after tamoxifen treatment and fed HFD for 12 weeks, compared to Oil control fed on HFD (B). Hemoglobin A1c level show improved A1C in smad2 knockout mice, three weeks after tamoxifen treatment (C).

FIGS. 14A-14D. IPGTT showed improvement in glucose clearance following smad2 knockout in adult beta cells 11 weeks after tamoxifen treatment (A). Homeostatic Model Assessment for Insulin Resistance (HOMA-IR) show improved insulin resistance in smad2 knockout mice (B). In vivo GSIS show improvement in insulin secretion (C). ITT performed fifteen weeks after tamoxifen treatment show improved insulin resistance in smad2 knockout mice fed HFD for 12 weeks compared to Oil control fed on HFD (D).

FIGS. 15A-15D. Repeat intervention restores glucose homeostasis in smad2 knockout mice maintained on a high fat diet (HFD). IPGTT showed improved glucose clearance one week after the second dose of tamoxifen in smad2 knockout mice (A). In vivo GSIS show improvement in insulin secretion one week after the second dose (B). IPGTT showed improved glucose clearance nine weeks after the second dose of tamoxifen in smad2 knockout mice (C). In vivo GSIS showed improvement in insulin secretion nine weeks after the second dose of tamoxifen (D).

FIG. 16. Adgene backbone containing the Smad2 SagRNA and rat insulin promoter.

FIG. 17. Crispr Cas9 targeting strategy that targets exon-4, exon-8 and exon-10 using the three sgRNA constructs (SEQ ID NOs: 22-24).

FIGS. 18A-18C. Crispr-Cas9 mediated knockdown of smad2 in beta-TC and Min-6 cells. Realtime PCR showed Crispr-Cas9 mediated smad2 knockdown in beta-TC cells (A). Realtime PCR showed Crispr-Cas9 mediated smad2 knockdown in MIN-6 cells (B). Primer sequences (SEQ ID NOs: 26-31) used in the experiments (C).

FIGS. 19A-19C. AAV6-GFP-U6-mSMAD2_shRNA treatment in vitro blocks smad2 expression in MIN6 and Beta-TC cell lines. Min6 and beta-TC cells sorted after transfection showed near complete ablation of smad2 expression (A). Phospho smad2 expression is nearly absent in Min6 cells after AAV6-GFP-U6-mSMAD2_shRNA treatment (B). RT-PCR quantification of MIN6 mRNA after treated with AAV6-GFP-U6-mSMAD2_shRNA show complete absence of mRNA in sorted cells (C). Similar experiments can be performed in human beta cell lines (EndoC-bH5, Human Cell Design) and primary human beta cells.

### SEQUENCES

The nucleic and amino acid sequences are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [Sequence_Listing, Apr. 20, 2022, 19.58 KB], which is incorporated by reference herein. In the accompanying sequence listing:
SEQ ID NOs: 1-4 are the nucleic acid sequences of shRNAs.
SEQ ID NOs: 5-11 are the nucleic acid sequences of promoters.
SEQ ID NO: 12 is an amino acid sequence of an exemplary Cas9.
SEQ ID NO: 13 is a nucleic acid sequence of a tracrRNA.
SEQ ID NOs: 14-20 are nucleic acid sequences that is transcribed to a crRNA.
SEQ ID NO: 21 is a nucleic acid sequence of a U6 promoter.
SEQ ID NO: 22 is a nucleic acid sequence that is transcribed to a a U6 gRNA.
SEQ ID NO: 23 is a nucleic acid sequence that is transcribed to a a tracrRNA.
SEQ ID NOs: 24-26 are nucleic acid sequences of targeted smad2 genes.
SEQ ID NO: 27 is a nucleic acid sequence of a DNA that is transcribed to a control RNA.
SEQ ID NOs: 28-33 are the nucleic acid sequences of PCR primers.
SEQ ID NO: 34 is the nucleic acid sequence of AAV6-GFP-U6-m-SMAD2-shRNA.
SEQ ID NO: 35 is a targeting nucleic acid sequence.

### DETAILED DESCRIPTION OF SEVERAL EMBODIMENTS

TGF-β/smad signaling involves binding of TGF-β-superfamily ligands (e.g. TGF-βs 1,2,3, activins, inhibins) to transmembrane-receptors that in turn activate receptor-regulated smads (R-smads, including smad2 and smad3) (Attisano and Lee-Hoeflich, Genome Biol. 2001; 2(8):REVIEWS3010). The activated R-smads form a complex with the common smad (smad4) and translocate to the nucleus to regulate the transcription of target genes (Matsuzaki, Cytokine Growth Factor Rev. 2013; 24(4):385-99; Wrighton et al., Cell Res. 2009; 19(1):8-20). In the pancreas, TGF-β/smad2 signaling regulates endocrine maturation and development; inhibiting smad2 during pancreas development leads to an increased proliferation and expansion of double-hormone-positive immature endocrine cells with reduced endocrine differentiation within the embryonic endocrine compartment (El-Gohary et al., Dev Biol. 2013; 378(2):83-93). In the adult islet, suppression of TGF-β signaling increases β-cell proliferation and mice with pancreas-specific smad2 and -3 gene ablation have robust β-cell proliferation after a non-diabetogenic loss of β-cells (60% partial pancreatectomy) (El-Gohary et al., Diabetes. 2014; 63(1):224-36). TGF-β signaling suppression increases β-cell proliferation after pancreatic duct ligation (El-Gohary et al., Diabetes. 2014; 63(1):224-36). In addition, suppression of TGF-β signaling promotes β-cell proliferation in both mouse pancreas and in human islets transplanted into NOD-scid IL-2Rg^{null} mice (Dhawan et al., Diabetes. 2016; 65(5):1208-18). In addition to a regulatory role in endocrine differentiation and proliferation, insulin expression and release is negatively regulated by TGF-β signaling (Carter et al., Biol Proced Online. 2009; 11:3-31; Matsumura et al., Biochem Biophys Res Commun. 2007; 364(1):151-6; Wang et al., Cell Metab. 2019; 29(3):638-52 e5). It is disclosed herein that (3-cell specific deletion of smad2 expression affects β-cell function and proliferation.

### Terms

Unless otherwise noted, technical terms are used according to conventional usage. Definitions of many common terms in molecular biology may be found in Krebs et al. (eds.), Lewin's genes XII, published by Jones & Bartlett Learning, 2017. The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Unless otherwise indicated, “about” means within five percent. Dates of GENBANK® Accession Nos. referred to herein are the sequences available at least as early as Dec. 31, 2019. All references, patent applications and publications, and GENBANK® Accession numbers cited herein are incorporated by reference.

In order to facilitate review of the various embodiments of this disclosure, the following explanations of specific terms are provided:

Alpha (α) cells: Mature glucagon producing endocrine cells. In vivo, these cells are found in the pancreatic islets of Langerhans.

Beta (β) cells: Mature insulin producing endocrine cells. In vivo, these cells are found in the pancreatic islets of Langerhans,

Delta (δ) cells: Mature somatostatin producing endocrine cells. In vivo, these cells are found in the pancreatic islets of Langerhans.

PP cells: Mature pancreatic polypeptide (PP) producing endocrine cells. In vivo, these cells are found in the pancreatic islets of Langerhans.

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 11 recognized serotypes of AAV (AAV1-11).

Administration: To provide or give a subject an agent by any effective route. Exemplary routes of administration include, but are not limited to, oral, injection (such as subcutaneous, intramuscular, intradermal, intraperitoneal, intravenous, and intraductal), sublingual, rectal, transdermal, intranasal, vaginal and inhalation routes. In some embodiments, administration is to a pancreatic duct.

Agent: Any polypeptide, compound, small molecule, organic compound, salt, polynucleotide, or other molecule of interest. Agent can include a therapeutic agent, a diagnostic agent or a pharmaceutical agent. A therapeutic agent is a substance that demonstrates some therapeutic effect by restoring or maintaining health, such as by alleviating the symptoms associated with a disease or physiological disorder, or delaying (including preventing) progression or onset of a disease, such as T2DM. An agent can be a viral vector including a nucleotide that is transcribed to form an inhibitory RNA.

Anti-diabetic lifestyle modifications: Changes to lifestyle, habits, and practices intended to alleviate the symptoms of diabetes or pre-diabetes. Obesity and sedentary lifestyle may both independently increase the risk of a subject developing T2DM, so anti-diabetic lifestyle modifications include those changes that will lead to a reduction in a subject's body mass index (BMI), increase physical activity, or both. Specific, non-limiting examples include the lifestyle interventions described in Diabetes Care, 22(4):623-34 at pages 626-27, herein incorporated by reference.

Clustered regularly interspaced short palindromic repeats (CRISPR) associated protein 9 (Cas9): An RNA-guided DNA endonuclease enzyme associated with the CRISPR (Clustered Regularly Interspersed Palindromic Repeats) adaptive immunity system in Streptococcus pyogenes, among other bacteria. Cas9 can cleave nearly any sequence complementary to the guide RNA. Includes Cas9 nucleic acid molecules and proteins. Cas9 sequences are publicly available, for example from the GENBANK® sequence database (e.g., Accession Nos. NP_269215.1 and AKS40378.1 provide exemplary Cas9 protein sequences, while Accession No. NC_002737.2 provides an exemplary Cas9 nucleic acid sequence therein). One of ordinary skill in the art can identify additional Cas9 nucleic acid and protein sequences, including Cas9 variants.

Diabetes mellitus: A group of metabolic diseases in which a subject has high blood sugar, either because the pancreas does not produce enough insulin, or because cells do not respond to the insulin that is produced. Type 1 diabetes mellitus (T1DM) results from the body's failure to produce insulin. This form has also been called “insulin-dependent diabetes mellitus” (IDDM) or “juvenile diabetes.” T1DM is characterized by loss of the insulin-producing R cells, leading to insulin deficiency. This type can be further classified as immune-mediated or idiopathic. T2DM results from insulin resistance, a condition in which cells fail to use insulin properly, sometimes combined with an absolute insulin deficiency. This form is also called “non insulin-dependent diabetes mellitus” (NIDDM) or “adult-onset diabetes.” The defective responsiveness of body tissues to insulin is believed to involve the insulin receptor. This disease is characterized by high blood sugar, insulin resistance, and relative lack of insulin. Common symptoms include increased thirst, frequent urination, and unexplained weight loss. T2DM makes up about 90% of the cases of diabetes, and can occur as a result of obesity, lack of exercise, and/or genetic predisposition. Treatment involves lifestyle modifications, including diet and exercise. Anti-diabetic medications of use include metformin. Other classes of medications include sulfonylureas, thiazolidinediones, dipeptidyl peptidase-4 inhibitors, SGLT2 inhibitors, and glucagon-like peptide-1 analogs.

Diabetes mellitus is characterized by recurrent or persistent hyperglycemia, and is diagnosed by demonstrating any one of:
a. Fasting plasma glucose level ≥7.0 mmol/1 (126 mg/dl);
b. Plasma glucose ≥11.1 mmol/1 (200 mg/dL) two hours after a 75 g oral glucose load as in a glucose tolerance test;
c. Symptoms of hyperglycemia and casual plasma glucose ≥11.1 mmol/1 (200 mg/dl);
d. Glycated hemoglobin (Hb A1C) ≥6.5%.

Donor polynucleotide: A polynucleotide that is capable of specifically inserting into a genomic locus.

Downstream: A relative position on a polynucleotide, wherein the “downstream” position is closer to the 3′ end of the polynucleotide than the reference point. In the instance of a double-stranded polynucleotide, the orientation of 5′ and 3′ ends are based on the sense strand, as opposed to the antisense strand.

Endocrine: Tissue which secretes regulatory hormones directly into the bloodstream without the need for an associated duct system.

Enhancer: A nucleic acid sequence that increases the rate of transcription by increasing the activity of a promoter.

Expand: A process by which the number or amount of cells is increased due to cell division. Similarly, the terms “expansion” or “expanded” refers to this process. The terms “proliferate,” “proliferation” or “proliferated” may be used interchangeably with the words “expand,” “expansion,” or “expanded.”

Expressed: Translation of a nucleic acid into a protein. Proteins may be expressed and remain intracellular, become a component of the cell surface membrane, or be secreted into the extracellular matrix or medium.

Exocrine: Secretory tissue which distributes its products, such as enzymes, via an associated duct network. The exocrine pancreas is the part of the pancreas that secretes enzymes required for digestion. The exocrine cells of the pancreas include the centroacinar cells and basophilic cells, which produce secretin and cholecystokinin.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Heterologous: A heterologous sequence is a sequence that is not normally (in the wild-type sequence) found adjacent to a second sequence. In one embodiment, the sequence is from a different genetic source, such as a virus or organism, than the second sequence.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Inhibitory nucleic acid molecules: Includes inhibitory RNA and DNA molecules, such as an antisense oligonucleotide, a small interfering RNA (siRNA), a microRNA (miRNA), a short hairpin (shRNA) or a ribozyme. Any type of inhibitory nucleic acid molecule that specifically targets and regulates expression of a nucleic acid encoding smad2 is contemplated for use. A smad2 antisense compound is one which specifically hybridizes with and modulates expression of a smad2 nucleic acid molecule. These compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self-complementarity to allow for hybridization and formation of a fully or partially double-stranded compound. In further examples, a shRNA is an RNA oligonucleotide that forms a tight hairpin, which is cleaved into siRNA. siRNA molecules are generally about 15-40 nucleotides in length, such as 20-25 nucleotides in length, and may have a 0 to 5 nucleotide overhang on the 3′ or 5′ end, or may be blunt ended. Generally, one strand of a siRNA is at least partially complementary to a nucleic acid molecule encoding smad2 protein.

Inhibiting or treating a disease: Inhibiting a disease, such as diabetes, refers to inhibiting the full development of a disease. In several examples, inhibiting a disease refers to lessening symptoms of the disease, such as diabetes. “Treatment” refers to a therapeutic intervention that ameliorates a sign or symptom of a disease or pathological condition related to the disease, such as glucose intolerance.

Inhibitor: An agent that decreases expression or function, such as of smad2. Inhibition does not need to be complete for an agent to be an inhibitor. In some embodiments, an inhibitor can decrease expression or function of smad2 by about 30%, 40%, 50%, 60%, 70%, 80%, 90%, 95%, or 100%. Inhibitors include inhibitory RNAs, small molecules and CRISPR/Cas9 systems.

Insulin: A protein hormone involved in the regulation of blood sugar levels that is produced by pancreatic beta cells. In vivo, insulin is produced as a precursor proinsulin, consisting of the B and A chains of insulin linked together via a connecting C-peptide. Insulin itself includes only the B and A chains. Exemplary insulin sequences are provided in GENBANK® Accession NO. NM_000207.2 (human) and NM_008386.3 (mouse), as available on Apr. 1, 2015, and are incorporated by reference herein. Exemplary nucleic acid sequences encoding insulin are provided in GENBANK® Accession No: NM_000207.2 (human) and NM_008386.3 (mouse), as available on Apr. 1, 2015, and are incorporated by reference herein. The term insulin also encompasses species variants, homologues, allelic forms, mutant forms, and equivalents thereof, including conservative substitutions, additions, deletions therein not adversely affecting the structure of function.

Islets of Langerhans: Small discrete clusters of pancreatic endocrine tissue. In vivo, in an adult mammal, the islets of Langerhans are found in the pancreas as discrete clusters (islands) of pancreatic endocrine tissue surrounded by the pancreatic exocrine (or acinar) tissue. In vivo, the islets of Langerhans consist of the a cells, R cells, 6 cells, PP cells, and F cells. Histologically, in rodents, the islets of Langerhans consist of a central core of R cells surrounded by an outer layer of α cells, δ cells, and PP cells. The structure of human islets of Langerhans is different and distinct from rodents. The islets of Langerhans are sometimes referred to herein as “islets.”

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

Mothers against decapentaplegic homolog (smad)2: A protein also known as SMAD family member 2, and MAD homolog 2, encoded by the smad2 gene. Smad2 mediates the signal of the transforming growth factor (TGF)-β, and thus regulates multiple cellular processes, such as cell proliferation, apoptosis, and differentiation. Smad2 is recruited to the TGF-β receptors through its interaction with the smad anchor for receptor activation (SARA) protein. In response to TGF-β signal, smad2 is phosphorylated by the TGF-β receptors. The phosphorylation induces the dissociation of smad2 with SARA and the association with the family member smad4. The association with smad4 is important for the translocation of smad2 into the cell nucleus, where it binds to target promoters and forms a transcription repressor complex with other cofactors. Smad2 can also be phosphorylated by activin type 1 receptor kinase, and mediates the signal from the activin. An exemplary nucleic acid encoding human smad2 is disclosed in NCBI Ref. Seq. No. NM_001003652.4, Feb. 20, 2021, incorporated herein by reference, and an exemplary protein sequence is disclosed in NCBI Ref. Seq. No. NP_001003652.1, Feb. 20, 2021, both incorporated herein by reference. An exemplary nucleic acid sequence encoding murine smad2 is disclosed in NCBI Ref. Seq. No. NM_001252481.1, Mar. 16, 2021, and an exemplary protein sequence for murine smad2 is disclosed in NCBI Ref. Seq. No. NP_001239410, Mar. 16, 2021, both incorporated herein by reference.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes.

Lentivirus: A genus of retroviruses. The virions are enveloped, slightly pleomorphic, spherical and measure 80-100 nm in diameter. Projections of envelope make the surface appear rough, or tiny spikes (about 8 nm) may be dispersed evenly over the surface. Lentiviruses have gag, pol and env genes, coding for viral proteins in the order: 5′-gag-pol-env-3′, similar to all other retroviruses. However, unlike other retroviruses, lentivirus genomes also encode two regulatory genes, tat and rev, and may also have additional accessory genes depending on the virus (e.g., for HIV-1: vif, vpr, vpu, nef) whose products are involved in regulation of synthesis and processing viral RNA and other replicative functions. The Long terminal repeat (LTR) is about 600 nt long, of which the U3 region is 450, the R sequence 100 and the U5 region some 70 nt long. The structural proteins of lentiviruses include the gp120 surface envelope protein, gp41 transmembrane envelope protein, p24 capsid protein, p17 matrix protein, and p7/p9 capsid protein. Enzymes encoded by lentiviruses include reverse transcriptase, integrase, protease, and dUTPase. Gene regulatory proteins encoded by lentiviruses include tat and rev. Accessory proteins encoded by lentiviruses include nef, vpr, vif, vpu/vpx, and pg.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

A “norpholino” oligonucleotide refers to a polymeric molecule having a backbone which supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen (see, U.S. Pat. No. 7,888,012, incorporated herein by reference).

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. “Transcribed to form” indicates that the DNA is transcribed into an RNA, such as, but not limited to, an inhibitory nucleic acid molecule. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, for example, Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, such as version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (http://www.ncbi.nlm.nih.gov/). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

ORF (open reading frame): A series of nucleotide triplets (codons) coding for amino acids without any termination codons. These sequences are usually translatable into a peptide.

Pancreatic endocrine cell: An endocrine cell of pancreatic origin that produces one or more pancreatic hormone, such as insulin, glucagon, somatostatin, or pancreatic polypeptide. Subsets of pancreatic endocrine cells include the a (glucagon producing), R (insulin producing) 6 (somatostatin producing) or PP (pancreatic polypeptide producing) cells. In some embodiments, pancreatic endocrine cells produce ghrelin. Additional subsets produce more than one pancreatic hormone, such as, but not limited to, a cell that produces both insulin and glucagon, or a cell that produces insulin, glucagon, and somatostatin, or a cell that produces insulin and somatostatin.

Pharmaceutically acceptable carriers: The pharmaceutically acceptable carriers useful in this invention are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or a composition capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Pre-diabetes: A state in which some, but not all, of the criteria for diabetes are met. For example, a subject can have impaired fasting glycaemia or impaired fasting glucose (IFG). Subjects with fasting glucose levels of 100 or higher but less than 126 mg/dl (6.1 to 6.9 mmol/1) are considered to have impaired fasting glucose. Subjects with plasma glucose at or above 140 mg/dL (7.8 mmol/L), but not over 200 mg/dL (11.1 mmol/L), two hours after a 75 g oral glucose load are considered to have impaired glucose tolerance. Subjects with an elevated HbA1c level (5.7%-6.5%) are considered pre-diabetic. Pre-diabetes can be diagnosed by:
A1C 5.7% to <6.5%
Impaired fasting glucose: fasting glucose ≥100 but <126 mg/dL.
Impaired glucose tolerance: 2-h plasma glucose ≥140 but <200 mg/dL during an OGTT, when the test is performed as described by the World Health Organization, using a glucose load containing the equivalent of 1.75 mg/kg (max 75 g) anhydrous glucose dissolved in water.

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell. An “epitope” is a region of a polypeptide capable of binding an immunoglobulin generated in response to contact with an antigen. Thus, smaller peptides containing the biological activity of insulin, or conservative variants of the insulin, are thus included as being of use.

The term “soluble” refers to a form of a polypeptide that is not inserted into a cell membrane.

The term “substantially purified polypeptide” as used herein refers to a polypeptide which is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative substitutions replace one amino acid with another amino acid that is similar in size, hydrophobicity, etc. Variations in the cDNA sequence that result in amino acid changes, whether conservative or not, should be minimized in order to preserve the functional and immunologic identity of the encoded protein. The immunologic identity of the protein may be assessed by determining if it is recognized by an antibody; a variant that is recognized by such an antibody is immunologically conserved. Any cDNA sequence variant can introduce no more than twenty, such as fewer than ten amino acid substitutions into the encoded polypeptide. Variant amino acid sequences may, for example, be 80, 90 or even 95% or 98% identical to the native amino acid sequence.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). A promoter that is “macrophage specific” is increased expression in macrophage cells as compared to other cell types, such as, but not limited to, lymphocytes, natural killer cells and neutrophils.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, such as by genetic engineering techniques. Similarly, a recombinant protein is one encoded for by a recombinant nucleic acid molecule. In addition, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” refers to an AAV particle in which a recombinant nucleic acid molecule (such as a recombinant nucleic acid molecule encoding Pdx1 and MafA) has been packaged.

Recombination: A process of exchange of genetic information between two polynucleotides. “Homologous recombination (HR)” refers to the specialized form of an exchange that takes place, for example, during repair of double-strand breaks in cells. Nucleotide sequence homology is utilized in recombination, for example using a “donor” molecule to template repair of a “target” molecule (i.e., the one that experienced the double-strand break), and is variously known as “non-crossover gene conversion” or “short tract gene conversion,” because it leads to the transfer of genetic information from the donor to the target.

Selectively hybridize: Hybridization under moderately or highly stringent conditions that excludes non-related nucleotide sequences.

In nucleic acid hybridization reactions, the conditions used to achieve a particular level of stringency will vary, depending on the nature of the nucleic acids being hybridized. For example, the length, degree of complementarity, nucleotide sequence composition (for example, GC v. AT content), and nucleic acid type (for example, RNA versus DNA) of the hybridizing regions of the nucleic acids can be considered in selecting hybridization conditions. An additional consideration is whether one of the nucleic acids is immobilized, for example, on a filter.

A specific example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can determine variations on these conditions (e.g., Molecular Cloning: A Laboratory Manual, 2nd ed., vol. 1-3, ed. Sambrook et al., Cold Spring Harbor Laboratory Press, Cold Spring Harbor, N Y, 1989). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Sequence identity of amino acid sequences: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of proteins are typically characterized by possession of at least about 75%, for example at least about 80%, 90%, 95%, 96%, 97%, 98% or 99% sequence identity counted over the full length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: Any mammal, such as humans, non-human primates, pigs, sheep, cows, rodents and the like which is to be the recipient of the particular treatment. In two non-limiting examples, a subject is a human subject or a murine subject. In some embodiments, the subject has T2DM.

Therapeutic agent: Used in a generic sense, it includes treating agents, prophylactic agents, and replacement agents. A therapeutic agent can be a smad2 inhibitor.

Therapeutically effective amount: A quantity of a specified pharmaceutical or therapeutic agent (e.g. a smad2 inhibitor) sufficient to achieve a desired effect in a subject, or in a cell, being treated with the agent, such as increasing insulin production or glucose responsiveness in a subject with diabetes. The effective amount of the agent will be dependent on several factors, including, but not limited to the subject or cells being treated, and the manner of administration of the therapeutic composition.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses {Wolff, J. A., ed, Gene Therapeutics, Birkhauser, Boston, USA (1994)}. In the case of infection by retroviruses, the infecting retrovirus particles are absorbed by the target cells, resulting in reverse transcription of the retroviral RNA genome and integration of the resulting provirus into the cellular DNA. Methods for the introduction of genes into the pancreatic endocrine cells are known (e.g. see U.S. Pat. No. 6,110,743, herein incorporated by reference). These methods can be used to transduce a pancreatic endocrine cell produced by the methods described herein, or an artificial islet produced by the methods described herein.

Genetic modification of the target cell is an indicium of successful transfection. “Genetically modified cells” refers to cells whose genotypes have been altered as a result of cellular uptakes of exogenous nucleotide sequence by transfection. A reference to a transfected cell or a genetically modified cell includes both the particular cell into which a vector or polynucleotide is introduced and progeny of that cell.

Transgene: An exogenous gene supplied by a vector.

Upstream: A relative position on a polynucleotide, wherein the “upstream” position is closer to the 5′ end of the polynucleotide than the reference point. In the instance of a double-stranded polynucleotide, the orientation of 5′ and 3′ ends are based on the sense strand, as opposed to the antisense strand.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like. In some embodiments herein, the vector is an adenovirus vector or an AAV vector.

Virus: Microscopic infectious organism that reproduces inside living cells. A virus consists essentially of a core of a single nucleic acid surrounded by a protein coat and has the ability to replicate only inside a living cell. “Viral replication” is the production of additional virus by the occurrence of at least one viral life cycle. Viral vectors are known in the art, and include, for example, adenovirus, AAV, lentivirus and herpes virus.

It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

### Smad2 Inhibitors

It is disclosed herein that smad2 inhibitors are of use for treating T2DM. The smad2 inhibitor can be, for example, an inhibitory nucleic acid molecule, such as a small interfering (si)RNA, a short hairpin (sh)RNA, or a ribozyme. The inhibitor can be a small molecule. A smad2 inhibitor can be administered to a subject to treat T2DM.

### A. Inhibitory Nucleic Acid Molecules

Inhibitory nucleic acids that decrease the expression and/or activity of smad2 protein can be used in the methods disclosed herein. In some examples, such inhibitor nucleic acid molecules decrease smad2 protein expression or activity by at least 20%, at least 40%, at least 50%, at least 75%, at least 80%, at least 90%, at least 95%, at least 98% or even 100%. One embodiment is an RNA interference (RNAi), such as, but not limited to, small interfering RNA (siRNA) or short hairpin RNA (shRNA), which can be used for interference or inhibition of expression of a target gene. RNAs that specifically target smad2, such as siRNA and shRNA, are commercially available, for example from Santa Cruz Biotechnology, Inc., ThermoFisher Scientific, and Origene.

Generally, siRNAs are generated by the cleavage of relatively long double-stranded RNA molecules by Dicer or DCL enzymes (Zamore, Science, 296:1265-1269, 2002; Bernstein et al., Nature, 409:363-366, 2001). In animals and plants, siRNAs are assembled into RISC and guide the sequence specific ribonucleolytic activity of RISC, thereby resulting in the cleavage of mRNAs or other RNA target molecules in the cytoplasm. In the nucleus, siRNAs also guide heterochromatin-associated histone and DNA methylation, resulting in transcriptional silencing of individual genes or large chromatin domains. shRNAs, as opposed to siRNAs, are synthesized in the nucleus of cells, further processed and transported to the cytoplasm. shRNA can be transcribed by either RNA polymerase II or III through RNA polymerase II or III promoters on the expression cassette. The primary transcript generated from RNA polymerase TT promoter contains a hairpin like stem-loop structure that is processed in the nucleus. The steps of processing of a shRNA are disclosed, for example, in Rao et al., Adv. Drug. Del. Rev. 61: 746-759, 2009. Exemplary DNA that are transcribed to form shRNAs of use include, but are not limited to:

pEnter-RIP-mir30-shRNA_smad2
(SEQ ID NO: 1)
GACGGTTAGATGAGCTTGAGA
pEnter-RIP-mir30-shRNA_smad2
(SEQ ID NO: 2)
TGGTGTTCAATCGCATACTAT
pEnter-RIP-mir30-shRNA_smad2
(SEQ ID NO: 3)
GCTGAACTGTCTCCTACTACT
pEnter-RIP-mir30-shRNA_scramble
(SEQ ID NO: 4)
GCGCGATAGCGCTAATAATTT
DNA transcribed to form shRNA

The present disclosure utilizes RNA suitable for interference or inhibition of expression of smad2 protein, which RNA includes double stranded RNA of about 19 to about 40 nucleotides with the sequence that is substantially identical to a portion of an mRNA or transcript of a target gene, such as smad2 protein, for which interference or inhibition of expression is desired. For purposes of this disclosure, a sequence of the RNA “substantially identical” to a specific portion of the mRNA or transcript of the target gene for which interference or inhibition of expression is desired differs by no more than about 30%, and in some embodiments no more than about 10% or no more than 5% from the specific portion of the mRNA or transcript of the target gene. In particular embodiments, the sequence of the RNA is exactly identical to a specific portion of the mRNA or transcript of the target gene (e.g., smad2 protein transcripts).

Thus, siRNAs disclosed herein include double-stranded RNA of about 15 to about 40 nucleotides in length and a 3′ or 5′ overhang having a length of 0 to 5-nucleotides on each strand, wherein the sequence of the double stranded RNA is substantially identical to (see above) a portion of a mRNA or transcript of a nucleic acid encoding smad2 protein. In particular examples, the double stranded RNA contains about 19 to about 25 nucleotides, for instance 20, 21, or 22 nucleotides substantially identical to a nucleic acid encoding smad2 protein. In additional examples, the double stranded RNA contains about 19 to about 25 nucleotides 100% identical to a nucleic acid encoding smad2 protein. It should be noted that in this context “about” refers to integer amounts only. In one example, “about” 20 nucleotides refers to a nucleotide of 19 to 21 nucleotides in length.

Regarding the overhang on the double-stranded RNA, the length of the overhang is independent between the two strands, in that the length of one overhang is not dependent on the length of the overhang on other strand. In specific examples, the length of the 3′ or 5′ overhang is 0-nucleotide on at least one strand, and in some cases it is 0-nucleotide on both strands (thus, a blunt dsRNA). In other examples, the length of the 3′ or 5′ overhang is 1-nucleotide to 5-nucleotides on at least one strand. More particularly, in some examples the length of the 3′ or 5′ overhang is 2-nucleotides on at least one strand, or 2-nucleotides on both strands. In particular examples, the dsRNA molecule has 3′ overhangs of 2-nucleotides on both strands.

Thus, in one particular embodiment, the double-stranded RNA contains 20, 21, or 22 nucleotides, and the length of the 3′ overhang is 2-nucleotides on both strands. In embodiments of the RNAs provided herein, the double-stranded RNA contains about 40-60% adenine+uracil (AU) and about 60-40% guanine+cytosine (GC). More particularly, in specific examples the double-stranded RNA contains about 50% AU and about 50% GC.

In some examples, an antisense oligonucleotide is a single stranded antisense compound, such that when the antisense oligonucleotide hybridizes to a mRNA encoding smad2 protein and the resulting duplex is recognized by RNaseH, resulting in cleavage of the mRNA. In some examples, a siRNA is a single-stranded RNA molecule, such as about 21-23 nucleotides in length that is at least partially complementary to an mRNA molecule that regulates gene expression through an RNAi pathway. Antisense compounds specifically targeting a smad2 gene can be prepared by designing compounds that are complementary to a target nucleotide sequence, such as an mRNA sequence.

Also disclosed herein are RNAs that further include at least one modified ribonucleotide, for instance in the sense strand of the double-stranded RNA. In particular examples, the modified ribonucleotide is in the 3′ overhang of at least one strand, or more particularly in the 3′ overhang of the sense strand. It is contemplated that examples of modified ribonucleotides include ribonucleotides that include a detectable label (for instance, a fluorophore, such as rhodamine or FITC), a thiophosphate nucleotide analog, a deoxynucleotide (considered modified because the base molecule is ribonucleic acid), a 2′-fluorouracil, a 2′-aminouracil, a 2′-aminocytidine, a 4-thiouracil, a 5-bromouracil, a 5-iodouracil, a 5-(3-aminoallyl)-uracil, an inosine, or a 2′O-Me-nucleotide analog.

Antisense and ribozyme molecules for smad2 protein are of use in the methods disclosed herein. Antisense nucleic acids are DNA or RNA molecules that are complementary to at least a portion of a specific mRNA molecule (Weintraub, Scientific American 262:40, 1990). In the cell, the antisense nucleic acids hybridize to the corresponding mRNA, forming a double-stranded molecule. The antisense nucleic acids interfere with the translation of the mRNA, since the cell will not translate an mRNA that is double-stranded. Antisense oligomers of about 15 nucleotides can be used, since they are easily synthesized and are less likely to cause problems than larger molecules when introduced into the target cell producing smad2 protein. The use of antisense methods to inhibit the in vitro translation of genes is known (see, for example, Marcus-Sakura, Anal. Biochem. 172:289, 1988).

An antisense oligonucleotide can be, for example, about 5, 10, 15, 20, 25, 30, 35, 40, 45 or 50 nucleotides in length. Antisense compounds need not be 100% complementary to the nucleic acid molecule encoding smad2 to specifically hybridize and regulate expression of the target. For example, the antisense compound, or antisense strand of the compound if a double-stranded compound, can be at least 75%, at least 80%, at least 85%, at least 90%, at least 95%, at least 99% or 100% complementary to a nucleic acid molecule encoding smad2. Methods of screening antisense compounds for specificity are known (see, for example, U.S. Publication No. 2003-0228689).

An antisense nucleic acid can be constructed using chemical synthesis and enzymatic ligation reactions. For example, an antisense nucleic acid molecule can be chemically synthesized using naturally occurring nucleotides or variously modified nucleotides designed to increase the biological stability of the molecules or to increase the physical stability of the duplex formed between the antisense and sense nucleic acids, such as phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine-e, 5-carboxymethylaminomethyluracil, dihydrouracil, beta-D-galactosylqueosine, inosine, amongst others.

An antisense compound can be a morpholino antisense compound. Such antisense compounds are substantially uncharged antisense compounds (i) composed of morpholino subunits and phosphorus-containing intersubunit linkages joining a norpholino nitrogen of one subunit to a 5′ exocyclic carbon of an adjacent subunit: (ii) capable of uptake by target beta cells in the subject; (iii) containing between 10-40 nucleotide bases; (iv) having a base sequence effective to hybridize to a nucleic acid encoding smad2 to form a heteroduplex complex having a Tm substantially greater than 37° C., such as at least 45° C., and (vi) nuclease resistance. Morpholino oligonucleotides, particularly phosphoramidate- or phosphorodiamidate-linked morpholino oligonucleotides have been shown to have high binding affinities for complementary or near-complementary nucleic acids. Morpholino oligormers also exhibit little or no non-specific antisense activity, afford good water solubility, are immune to nucleases, and are designed to have low production costs (Summerton and Weller 1997) . Morpholino oligonucleotides (including antisense oligomers) are detailed, for example, in co-owned U.S. Pat. Nos. 5,698,685, 5,217,866, 5,142,047, 5,034,506, 5,166,315, 5,185A44, 5,521,063, and 5,506,337, all of which are incorporated by reference herein.

Use of an oligonucleotide to stall transcription is known as the triplex strategy where an oligonucleotide winds around double-helical DNA, forming a three-strand helix. Therefore, these triplex compounds can be designed to recognize a unique site on a chosen gene (Maher, et al., Antisense Res. and Dev. 1(3):227, 1991; Helene, C., Anticancer Drug Design 6(6):569), 1991. This type of inhibitory oligonucleotide is also of use in the methods disclosed herein.

Ribozymes, which are RNA molecules possessing the ability to specifically cleave other single-stranded RNA in a manner analogous to DNA restriction endonucleases, are also of use. Through the modification of nucleotide sequences, which encode these RNAs, it is possible to engineer molecules that recognize specific nucleotide sequences in an RNA molecule and cleave it (Cech, J. Amer. Med. Assn. 260:3030, 1988). An advantage of this approach is that, because they are sequence-specific, only mRNAs with particular sequences are inactivated.

There are two basic types of ribozymes namely, tetrahymena-type (Hasselhoff, Nature 334:585, 1988) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-base recognition sequences are preferable to shorter recognition sequences.

Various delivery systems are known and can be used to administer the siRNAs and other inhibitory nucleic acid molecules as therapeutics. Such systems include, for example, encapsulation in liposomes, microparticles, microcapsules, nanoparticles, recombinant cells capable of expressing the therapeutic molecule(s) (see, e.g., Wu et al., J. Biol. Chem. 262, 4429, 1987), construction of a therapeutic nucleic acid as part of a retroviral or other vector, and the like, see below.

### B. Vectors

Disclosed herein are vectors, such as a viral vector, such as a retroviral vector (for example, a lentiviral vector), an adenoviral vector, or an adeno-associated vector (AAV) that encodes an inhibitory nucleic acid molecule. These vectors include a nucleotide acid molecule transcribed to form an inhibitory nucleic acid molecule (e.g. inhibitory RNA, such as siRNA and shRNA), operably linked to a beta cell specific promoter. These promoters include, but are not limited to, an insulin promoter, nkx6.1 promoter, nkx6.2 promoter, neuroD promoter, or a pax4 promoter. In some non-limiting examples, the promoter is an insulin promoter. Exemplary sequences for these promoters are provided below:

Insulin
(SEQ ID NO: 5)
FP015412 INS_1: +U EU:NC; range −200 to 50. From Transcription start site (TSS)
TCCTGAGGAAGAGGTGCTGACGACCAAGGAGATCTTCCCACAGACCCAGCACCAGGGAAA
TGGTCCGGAAATTGCAGCCTCAGCCCCCAGCCATCTGCCGACCCCCCCACCCCAGGCCCT
AATGGGCCAGGCGGCAGGGGTTGAGAGGTAGGGGAGATGGGCTCTGAGACTATAAAGCCA
GCGGGGGCCCAGCAGCCCTCAGCCCTCCAGGACAGGCTGCATCAGAAGAGGCCATCAAGC
AGGTCTGTTCC
Pdx-1
>hg38_encodeCcreCombined range = chr13:27919800-27920049 5′ pad = 0
3′pad = 0 strand = + repeatMasking = none
(SEQ ID NO: 6)
GAGATCAGTGCGGAGCTGTCAAAGCGAGCAGGGGTGGCGCCGGGAGTGGG
Nkx6.1
>hg38_dna range = chr13:84498401-84498650 5′pad = 0 3′pad = 0 strand = -
repeatMasking = none
>hg38_dna range = chr13:84498401-84498650 5′pad = 0 3′pad = 0 strand = -
repeatMasking = none
(SEQ ID NO: 7)
AGCTTAAATATCCTTCCATCACCTAATTTACATTCATAAAATTTTAGGTG
Nkx6.2
>hg38_dna range = chr10:132786098-132786347 5′pad = 0 3′pad = 0 strand = -
repeatMasking = none
(SEQ ID NO: 8)
GAGATAAATCGGGGCGAATTTACAGCCCGGGAGGCACCTGCCGCGCTAAT
NeuroD
>hg38_dna range = chr2:181680827-181681076 5′pad = 0 3′pad = 0 strand = -
repeatMasking = none
(SEQ ID NO: 9)
GCTTCTGCCTCTTTCACCTCTGTCCTACTTCCGGCGCGAAAGCAGGCCAC
Pax4
>hg38_dna range = chr7:127618093-127618342 5′pad = 0 3′pad = 0 strand = -
repeatMasking = none
(SEQ ID NO: 10)
TTGCCTGTCTGAGAATGGCTCTGTCCCTGCTTCCGGGTGGTAGGTGGACA
Rat Insulin
>FP001510 Ins1_1: +U EU:NC; range −400 to 200.
(SEQ ID NO: 11)
atccagctatcaatagaaactatgaaacagttccagggacaaagataccaggtccccaac
aactgcaactttctgggaaatgaggtggaaaatgctcagccaaggaaaaagagggcctta
ccctctctgggacaatgattgtgctgtgaactgcttcatcaggccatctggccccttgtt
aataatctaattaccctaggtctaagtagagttgttgacgtccaatgagcgctttctgca
gacttagcactaggcaagtgtttggaaattacagcttcagcccctctcgccatctgccta
cctacccctcctagagcccttaatgggccaaacggcaaagtccagggggcagagaggagg
tgctttggactataaagctagtggagacccagtaactcccAACCCTAAGTGACCAGCTAC
AATCATAGACCATCAGCAAGCAGGTATGTACTCTCCTGGGTGAGCCCGGTTCCCCCAGCC
AAAACTCTAGGGACTTTAGGAAGGATGTGGGTTCCTCTCTTACATGGACCTTTTCCTAGC
CTCAACCCTGCCTATCTTCCAGGTCATTGTTCCAACATGGCCCTGTGGATGCGCTTCCTG
C.
In SEQ ID NO: 11,

lower case indicates sequences upstream from the transcription start site, and uppercase indicates sequences downstream from the transcription start site.

The promoter can include, or consist of, one of SEQ ID NOs: 5-11. The promoter can be at least 95%, 96%, 97%, 98% or 99% identical to one of SEQ ID NOs: 5-11, provided the sequence retains the function of the corresponding promoter. The promoter can include a deletion or addition of 1, 2, 3, 4, or 5 nucleotides at one or both ends of one of SEQ ID NOs: 5-11, provided the nucleic acid sequence retains the function of the corresponding promoter.

In some examples, the viral vector transcribed to form the inhibitory nucleic acid can be replication-competent. For example, the viral vector can have a mutation (e.g., insertion of nucleic acid encoding the protomer) in the viral genome that attenuates, but does not completely block viral replication in host cells. Suitable vectors are known in the art, and include viral vectors such as retroviral, lentiviral, adenoviral vectors, and AAV. In specific, non-limiting examples, the vector is a lentiviral vector, gammaretroviral vector, self-inactivating retroviral vector, adenoviral vector, or adeno-associated vector (AAV).

Various viral vectors which can be utilized for nucleic acid-based therapy as taught herein include adenovirus or adeno-associated virus, herpes virus, vaccinia, or an RNA virus such as a retrovirus (including HVJ; see Kotani et al., Curr. Gene Ther. 4:183-194, 2004). In one embodiment, the retroviral vector is a derivative of a murine or avian retrovirus, or a human or primate lentivirus. Examples of retroviral vectors in which a foreign gene can be inserted include, but are not limited to: Moloney murine leukemia virus (MoMLV), Harvey murine sarcoma virus (HaMuSV), murine mammary tumor virus (MuMTV), and Rous Sarcoma Virus (RSV). In one embodiment, when the subject is a human, a vector such as the gibbon ape leukemia virus (GaLV) can be utilized. A pseudotyped retroviral vector can be utilized that includes a heterologous envelope gene. A lentiviral vector is also of use.

A number of additional retroviral vectors can incorporate multiple genes. All of these vectors can transfer or incorporate a gene for a selectable marker so that transduced cells can be identified and generated. By inserting a nucleic acid transcribed to form the inhibitory nucleic acid molecule into the viral vector, along with another gene which can serve as viral envelope protein and also can encode the ligand for a receptor on a specific target cell, for example, the vector is now target specific. Retroviral vectors can be made target specific by modifications of the envelope protein by attaching, for example, a sugar, a glycolipid, or a protein. In one specific, non-limiting example, targeting is accomplished by using an antibody to target the retroviral vector.

Since recombinant retroviruses are non-replicating by design, they require assistance in order to produce infectious vector particles. This assistance can be provided, for example, by using helper cell lines that contain plasmids encoding all of the structural genes of the retrovirus under the control of regulatory sequences within the long terminal repeat (LTR). These plasmids are missing a nucleotide sequence which enables the packaging mechanism to recognize an RNA transcript for encapsidation. Helper cell lines which have deletions of the packaging signal include, but are not limited to ψ2, PA317, and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively, NIH 3T3 or other tissue culture cells can be directly transfected with plasmids encoding the retroviral structural genes gag, pol and env, by conventional transfection methods. These cells are then transfected with the vector plasmid containing the genes of interest. The resulting cells release the retroviral vector into the culture medium.

Viral vectors of use also include adenovirus or adeno-associated virus (AAV). Defective viruses, that entirely or almost entirely lack viral genes, can be used. In some examples, the vector is an attenuated adenovirus vector, such as the vector described by Stratford-Perricaudet et al. (J. Clin. Invest., 90:626-630 1992; La Salle et al., Science 259:988-990, 1993); and a defective adeno-associated virus vector (Samulski et al., J. Virol., 61:3096-3101, 1987; Samulski et al., J. Virol., 63:3822-3828, 1989; Lebkowski et al., Mol. Cell. Biol., 8:3988-3996, 1988).

Adenoviral vectors and/or adeno-associated viral vectors can be used in the methods disclosed herein. AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency. In some embodiments the AAV DNA includes a nucleic acid transcribed to form an inhibitor RNA, operably linked to a beta cell specific promoter. Further provided are recombinant vectors, such as recombinant adenovirus vectors and recombinant adeno-associated virus (rAAV) vectors comprising a nucleic acid molecule disclosed herein. In some embodiments, the AAV is rAAV8 and/or AAV2. However, the AAV serotype can be any other suitable AAV serotype, such as AAV1, AAV2, AAV3, AAV4, AAV5, AAV6, AAV7, AAV9, AAV10, AAV11 or AAV12, or a hybrid of two or more AAV serotypes (such as, but not limited to AAV2/1, AAV2/7, AAV2/8 or AAV2/9). The vector can be an AAV3 or AAVDJ vector.

In some embodiments, a modified AAV vector that encodes a microRNA and smad2 shRNA molecule will be inserted into the adenoviral/AAV under the control of insulin or another beta-cell specific promoter. Without being bound by theory, the pre-shRNA is processed in the nucleus by Dicer and taken up by the RNA-induced silencing complex (RISC). The antisense strand guides RISC to a complementary mRNA sequence to silence either by cleaving the mRNA or repressing translation.

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21(4):583-593, 2008). In some embodiments, these elements are included in the AAV vector.

The left ORF of AAV contains the Rep gene, which encodes four proteins—Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008). In some embodiments, these elements are included in the AAV vector

Exemplary AAV of use are AAVDJ, AAV2, AAV3, AAV5, AAV6, AAV8 and AAV9. Adenovirus, AAV2 and AAV8 are capable of transducing cells in the pancreas. Thus, any AAV vector can be used in the methods disclosed herein. In some non-limiting examples, rAAV3, rAAVDJ, and rAAV9 vectors are of use. In other non-limiting examples, the vector is an rAAV3 or rAAVDJ vector.

In some embodiments, the AAV vector includes only the ITR and the gene of interest, specifically the macrophage promoter operably linked to the nucleic acid molecule transcribed to form an inhibitor RNA. In these embodiments, Rep and Cap are provided by a host cell, or on another vector, for in vitro production of the virus, but the resulting virus does not include nucleic acid molecules encoding Rep or Cap. In some embodiments, rAAV particles are generated by transfecting producer cells with a plasmid (AAV cis-plasmid) containing a cloned recombinant AAV genome composed of foreign DNA flanked by the 145 nucleotide-long AAV ITRs, and a separate construct expressing in trans the viral rep and cap genes. The adenovirus helper factors, such as E1A, E1B, E2A, E40RF6 and VA RNAs, can be provided by either adenovirus infection or transfecting into production cells a third plasmid that provides these adenovirus helper factors. In some embodiments, HEK293 cells are utilized. These are commonly used AAV production cells, which include the E1A/E1b gene; the helper factors that need to be provided are E2A, E40RF6 and VA RNAs. Methods, vectors, and cells of use, are disclosed, for example, in U.S. Pat. Nos. 6,566,118; 6,686,200; 6,924,128, 7,091,029, and 7,208,315, which are all incorporated herein by reference.

In some embodiments, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing a rAAV can be delivered to the packaging host cell in the form of any genetic element which transfer the sequences carried thereon. The selected genetic element may be delivered by any suitable method, including those described herein. The methods used to construct vectors are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al., J. Virol., 70:520-532 (1993), U.S. Pat. No. 5,478,745, and PCT Publication No. and WO 2005/033321, incorporated herein by reference. In some embodiments, selected AAV components can be readily isolated using techniques available to those of skill in the art from an AAV serotype, including AAV3 or AAVDJ. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GENBANK®.

Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. AAV6 preferentially infects macrophages. Because of the advantageous features of AAV, an rAAV are of use in the methods disclosed herein. However, this is not limiting.

AAV possesses several additional desirable features for a gene therapy vector, including the ability to bind and enter target cells, enter the nucleus, the ability to be expressed in the nucleus for a prolonged period of time, and low toxicity. AAV can be used to transfect cells, and suitable vector are known in the art, see for example, U.S. Published Patent Application No. 2014/0037585, incorporated herein by reference. Methods for producing rAAV suitable for gene therapy are well known in the art (see, for example, U.S. Published Patent Application Nos. 2012/0100606; 2012/0135515; 2011/0229971; and 2013/0072548; and Ghosh et al., Gene Ther 13(4):321-329, 2006), and can be utilized with the methods disclosed herein.

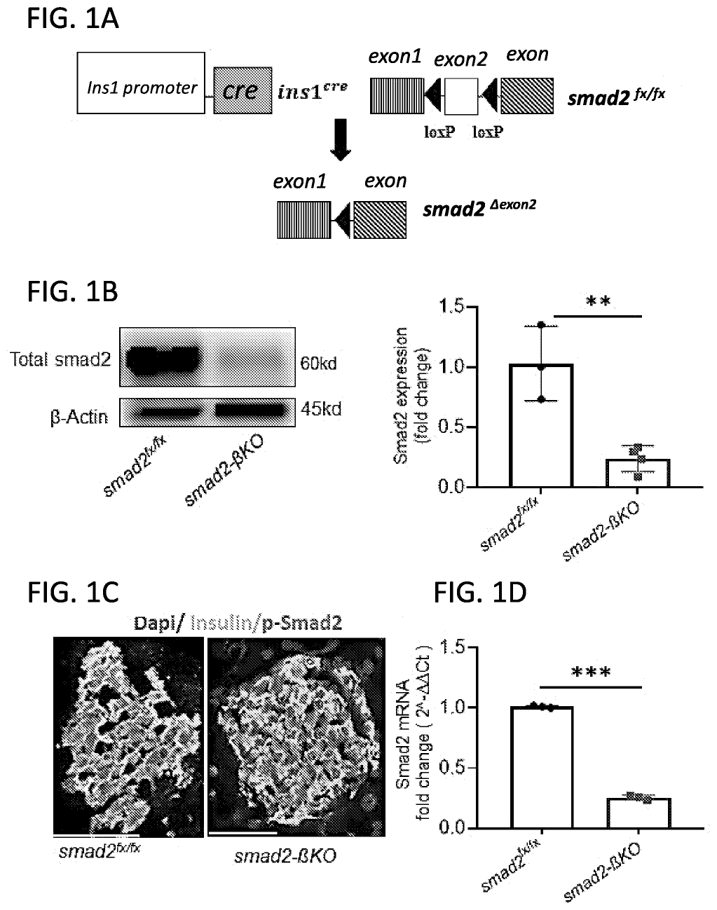

The vectors of use in the methods disclosed herein can contain nucleic acid sequences encoding an intact AAV capsid which may be from a single AAV serotype (e.g., AAVDJ, AAV2, AAV3, AAV, 6, AAV8 or AAV9). As disclosed in U.S. Pat. No. 6,156,303, vectors of use also can be recombinant, and thus can contain sequences encoding artificial capsids which contain one or more fragments of one AAV capsid fused to heterologous AAV or non-AAV capsid proteins (or fragments thereof). These artificial capsid proteins are selected from non-contiguous portions of a capsid from an AAV serotype of use. For example, a rAAV vector may have a capsid protein comprising one or more of the AAV3 capsid regions selected from the VP2 and/or VP3, or from VP1, or fragments thereof, see FIG. 1 of U.S. Pat. No. 6,156,303. In another example, it may be desirable to alter the start codon of the VP3 protein to GTG.

In some embodiments, a rAAV is generated having an AAV serotype 3 capsid. To produce the vector, a host cell which can be cultured that contains a nucleic acid sequence encoding an adeno-associated virus (AAV) serotype 3 capsid protein, or fragment thereof, as defined herein; a functional rep gene; a minigene composed of, at a minimum, AAV inverted terminal repeats (ITRs) and a transgene, such as a beta cell specific promoter, such as an insulin promoter, operably linked to a nucleic acid molecule transcribed to form the inhibitor RNA; and sufficient helper functions to permit packaging in the AAV3 capsid protein (or any other serotype of interest). The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. In some embodiments, a stable host cell will contain the required component(s) under the control of an inducible promoter. However, the required component(s) also can be under the control of a constitutive promoter.

In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing a rAAV can be delivered to the packaging host cell in the form of any genetic element which transfer the sequences carried thereon. The selected genetic element may be delivered by any suitable method, including those described herein. The methods used to construct vectors are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al., J. Virol., 70:520-532 (1993) and U.S. Pat. No. 5,478,745. In some embodiments, selected AAV components can be readily isolated using techniques available to those of skill in the art from an AAV serotype, including AAV3 or AAVDJ. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GENBANK®.

In some embodiments, the vector is a double-stranded self-complementary virus, or “scAAV vector.” scAAV vectors are disclosed in McCarty et al., 2001, Gene Ther. 8: 1248-1254; Carter PCT Publication No. WO 2001/011034; and Samulski, PCT Publication No. WO 2001/092551, all of which are incorporated by reference herein. As disclosed in PCT Publication No. “duplexed” DNA parvovirus vectors can be advantageously employed for gene delivery. Duplexed parvovirus can provide improved transduction to particle ratios, more rapid transgene expression, a higher level of transgene expression, and/or more persistent transgene expression. The duplexed parvovirus vectors can be used for gene delivery to host cells that are typically refractory to AAV transduction. Thus, duplexed parvovirus vectors, such as AAV3, can have a different host range than ssAAV (single-stranded) vectors.

These vectors are dimeric self-complementary (sc) polynucleotides (typically, DNA) packaged within a viral capsid, such as a parvovirus capsid, for example an AAV capsid, such as, but not limited to, AAV6. In some respects, the viral genome that is packaged within the capsid is essentially a “trapped” replication intermediate that cannot be resolved to produce the plus and minus polarity parvovirus DNA strands. Accordingly, the duplexed parvovirus vectors can circumvent the need for host cell mediated synthesis of complementary DNA inherent in conventional recombinant AAV (ssAAV) vectors.

This result is accomplished by allowing the virus to package essentially dimeric inverted repeats of the single-stranded parvovirus (e.g., ssAAV, such as ssAAV3) vector genome such that both strands, joined at one end, are contained within a single infectious capsid. Upon release from the capsid, the complementary sequences re-anneal to form transcriptionally active double-stranded DNA within the target cell.

The duplexed parvovirus vectors are fundamentally different from ssAAV vectors, and from the parent parvovirus in that the vDNA may form a double-stranded hairpin structure due to intrastrand base pairing, and that both DNA strands are encapsidated. Thus, the duplexed parvovirus vector is functionally similar to double-stranded DNA virus vectors rather than the parvovirus (e.g., ssAAV) from which it was derived.

The viral capsid may be from any parvovirus, either an autonomous parvovirus or dependovirus. In some embodiments, the viral capsid is an AAV capsid. The choice of parvovirus capsid may be based on a number of considerations as known in the art, e.g., the target cell type, the desired level of expression, the nature of the heterologous nucleotide sequence to be expressed, issues related to viral production, and the like.

The parvovirus particle may be a “hybrid” particle in which the viral terminal repeats (TRs) and viral capsid are from different parvoviruses. In some embodiments, the viral TRs and capsid are from different serotypes of AAV (e.g., as described in PCT Publication No. WO 00/28004 and Chao et al., Molecular Therapy 2:619, 2000; the disclosures of which are incorporated herein in entirety). In some embodiments, the virus has a “chimeric” capsid (e.g., containing sequences from different parvoviruses) or a “targeted” capsid (e.g., a directed tropism) as described in these publications. As used herein, a “duplexed parvovirus particle” encompasses hybrid, chimeric and targeted virus particles. In some embodiments, the duplexed parvovirus particle has an AAV capsid, which may further be a chimeric or targeted capsid.

A duplexed parvovirus vector can be produced by any suitable method. In some embodiments, the template for producing the vDNA is one that gives rise to a duplexed, rather than monomeric vDNA (i.e., the majority of vDNA produced are duplexed) which has the capacity to form a double-stranded vDNA. In some embodiments, at least about 50%, 60%, 70%, 80%, 90%, 95%, 98%, 99% or more of the replication products from the template are duplexed. In one embodiment, the template is a DNA molecule comprising one or more terminal repeat (TR) sequences. The template also comprises a modified TR that cannot be resolved (i.e., nicked) by the parvovirus Rep proteins. During replication, the inability of Rep protein to resolve the modified TR will result in a stable intermediate with the two “monomers” covalently attached by the non-resolvable TR. This “duplexed” molecule may be packaged within the parvovirus (AAV) capsid to produce a novel duplexed parvovirus vector, such as a scAAV6 vector.

While not wishing to be held to any particular theory, it is likely that the virion genome is retained in a single-stranded form while packaged within the viral capsid. Upon release from the capsid during viral infection, the dimeric molecule “snaps back” or anneals to form a double-stranded molecule by intra-strand base pairing, with the non-resolvable TR sequence forming a covalently-closed hairpin structure at one end. This double-stranded vDNA obviates host cell mediated second-strand synthesis, which may be a rate-limiting step for AAV transduction.

In some embodiments, the template further comprises a heterologous nucleotide sequence(s) to be packaged for delivery to a target cell. According to this particular embodiment, the heterologous nucleotide sequence is located between the viral TRs at either end of the substrate. In further preferred embodiments, the parvovirus (e.g., AAV) cap genes and rep genes are deleted from the template (and the vDNA produced therefrom). This configuration maximizes the size of the heterologous nucleic acid sequence(s) that can be carried by the parvovirus capsid. This can be the beta cell specific promoter operably linked to a nucleic acid molecule transcribed to form an inhibitory RNA.

In one embodiment, the template for producing the duplexed parvovirus vectors contains at least one TR at the 5′ and 3′ ends, flanking a heterologous nucleotide sequence of interest (such as the beta cell specific promoter operably linked to the nucleic acid molecule transcribed to form the inhibitory mRNA). The TR at one end of the substrate is non-resolvable, i.e., it cannot be resolved (nicked) by Rep protein. During replication, the inability of Rep protein to resolve one of the TRs will result in a stable intermediate with the two “monomers” covalently attached by the non-functional (i.e., non-resolvable) TR. The heterologous nucleotide sequence may be in either orientation with respect to the non-resolvable TR.

The term “flanked” is not intended to indicate that the sequences are necessarily contiguous. For example, in the example in the previous paragraph, there may be intervening sequences between the heterologous nucleotide sequence and the TR. A sequence that is “flanked” by two other elements, indicates that one element is located 5′ to the sequence and the other is located 3′ to the sequence; however, there may be intervening sequences between.

According to this embodiment, the template for producing the duplexed parvovirus vDNA is about half of the size of the wild-type (wt) parvovirus genome (e.g., AAV) corresponding to the capsid into which the vDNA will be packaged. In some embodiments, the template is from about 40% to about 55% of wt, such as from about 45% to about 52% of wt. Thus, the duplexed vDNA produced from this template can have a total size that is approximately the size of the wild-type parvovirus genome (e.g., AAV) corresponding to the capsid into which the vDNA will be packaged, e.g., from about 80% to about 105% of wt. In the case of AAV, the AAV capsid disfavors packaging of vDNA that substantially deviate in size from the wt AAV genome. In the case of an AAV capsid, the template can be approximately 5.2 kb in size or less. In other embodiments, the template is greater than about 3.6, 3.8, 4.0, 4.2, or 4.4 kb in length and/or less than about 5.4, 5.2, 5.0 or 4.8 kb in length.

In some embodiments, the heterologous nucleotide sequence(s) is less than about 2.5 kb in length (such as less than about 2.4 kb, for example less than about 2.2 kb in length, or less than about 2.1 kb in length) to facilitate packaging of the duplexed template by the parvovirus (e.g., AAV) capsid. In another embodiment, the template itself is duplexed, i.e., is a dimeric self-complementary molecule. According to this embodiment, the template comprises a resolvable TR at either end. The template further comprises a centrally-located non-resolvable TR. In some embodiments, each half of the template on either side of the non-resolvable TR is approximately the same length. Each half of the template (i.e., between the resolvable and non-resolvable TR) comprises one or more heterologous nucleotide sequence(s) of interest. The heterologous nucleotide sequence(s) in each half of the molecule is flanked by a resolvable TR and the central non-resolvable TR.

The sequences in either half of the template are substantially complementary (e.g., at least about 90%, 95%, 98%, 99% nucleotide sequence complementarity or more), so that the replication products from the template may form double-stranded molecules due to base-pairing between the complementary sequences. In other words, the template is essentially an inverted repeat with the two halves joined by the non-resolvable TR.

In some non-limiting examples, the heterologous nucleotide sequence(s) in each half of the template are essentially completely self-complementary (i.e., contains an insignificant number of mis-matched bases, or even no mismatched bases). In additional non-limiting examples, the two halves of the nucleotide sequence are essentially completely self-complementary.

The TR(s) (resolvable and non-resolvable) can be AAV sequences, such as serotypes 1, 2, 3, 4, 5, 6, 7, 8, or 9. The term “terminal repeat” includes synthetic sequences that function as an AAV inverted terminal repeat, such as the “double-D sequence” as described in U.S. Pat. No. 5,478,745, incorporated by reference. Resolvable AAV TRs need not have a wild-type TR sequence (e.g., a wild-type sequence may be altered by insertion, deletion, truncation or missense mutations), as long as the TR mediates the desired functions, such as virus packaging, integration, and/or provirus rescue, and the like. In some embodiments, the TRs are from the same parvovirus, e.g., both TR sequences are from AAV3 or another serotype of interest.

The viral Rep protein(s) used for producing the duplexed vectors are selected with consideration for the source of the viral TRs. For example, the AAV3 TR interacts more efficiently with the AAV3 Rep protein.

The genomic sequences of the various autonomous parvoviruses and the different serotypes of AAV, as well as the sequences of the TRs, capsid subunits, and Rep proteins are known in the art. Such sequences may be found in the literature or in public databases such as GENBANK®. See, e.g., GENBANK® Accession Numbers NC 002077, NC 001863, NC 001862, NC 001829, NC 001729, NC 001701, NC 001510, NC 001401, AF063497, U89790, AF043303, AF028705, AF028704, J02275, J01901, J02275, X01457, AF288061, AH009962, AY028226, AY028223, NC 001358, NC 001540; the disclosures of which are incorporated by references as available on Dec. 30, 2019. See also, e.g., Chiorini et al., (1999) J. Virology 73:1309; Xiao et al., (1999) J. Virology 73:3994; Muramatsu et al., (1996) Virology 221:208; PCT Publication Nos. WO 00/28061, WO 99/61601, WO 98/11244; and U.S. Pat. No. 6,156,303, all incorporated by reference herein.

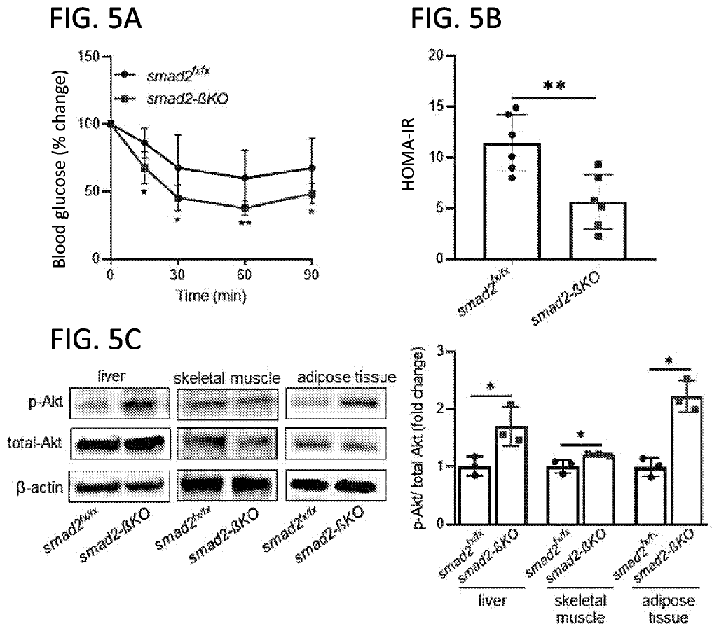

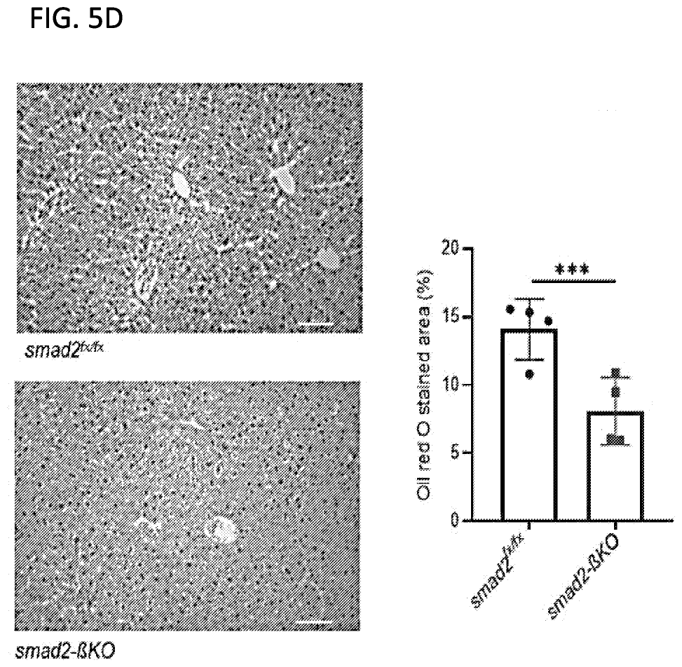

The non-resolvable TR may be produced by any method known in the art. For example, insertion into the TR will displace the nicking site (i.e., trs) and result in a non-resolvable TR. The designation of the various regions or elements within the TR are known in the art. An illustration of the regions within the AAV TR is provided in Fields et al., Virology, volume 2, chapter 69, FIG. 5, 3d ed., Lippincott-Raven Publishers. The insertion can be made into the sequence of the terminal resolution site (trs). Alternatively, the insertion may be made at a site between the Rep Binding Element (RBE) within the A element and the trs in the D element. The core sequence of the AAV trs site is known in the art and has been described by Snyder et al., (1990) Cell 60:105; Snyder et al., (1993) J. Virology 67:6096; Brister & Muzyczka, (2000) J. Virology 74:7762; Brister & Muzyczka, (1999) J. Virology 73:9325 (the disclosures of which are hereby incorporated by reference in their entireties). For example, Brister & Muzyczka, (1999) J. Virology 73:9325 describes a core trs sequence of 3′-CCGGT/TG-5* in the D element. Snyder et al., (1993) J. Virology 67:6096 identified the minimum trs sequence as 3′-GGT/TGA-5′, which substantially overlaps the sequence identified by Brister & Muzyczka. In some embodiments, the insertion is in the region of the trs site. The insertion may be of any suitable length that will reduce or substantially eliminate (e.g., by 60%, 70%), 80%. 90%, 95% or greater) resolution of the TR. In some embodiments, the insertion is at least about 3, 4, 5, 6, 10, 15, 20 or 30 nucleotides or more. There are no particular upper limits to the size of the inserted sequence, as long as suitable levels of viral replication and packaging are achieved (e.g., the insertion can be as long as 50, 100, 200 or 500 nucleotides or longer).

In another embodiment, the TR may be rendered non-resolvable by deletion of the trs site. The deletions can extend 1, 3, 5, 8, 10, 15, 20, 30 nucleotides or more beyond the trs site, as long as the template retains the desired functions. In addition to the trs site, some or all of the D element may be deleted. Deletions may further extend into the A element, however those skilled in the art will appreciate that it may be advantageous to retain the RBE in the A element, e.g., to facilitate efficient packaging. Deletions into the A element may be 2, 3, 4, 5, 8, 10, or 15 nucleotides in length or more, as long as the non-resolvable TR retains any other desired functions. It is further preferred that some or all of the parvovirus (e.g., AAV) sequences going beyond the D element outside the TR sequence (e.g., to the right of the D element) be deleted to prevent gene conversion to correct the altered TR.

As still a further alternative, the sequence at the nicking site may be mutated so that resolution by Rep protein is reduced or substantially eliminated. For example, A and/or C bases may be substituted for G and/or T bases at or near the nicking site. The effects of substitutions at the terminal resolution site on Rep cleavage have been described by Brister & Muzyczka, (1999) J. Virology 73:9325 (the disclosure of which is hereby incorporated by reference). As a further alternative, nucleotide substitutions in the regions surrounding the nicking site, which have been postulated to form a stem-loop structure, may also be used to reduce Rep cleavage at the terminal resolution site.

Those skilled in the art will appreciate that the alterations in the non-resolvable TR may be selected so as to maintain desired functions, if any, of the altered TR (e.g., packaging, Rep recognition, site-specific integration, and the like). In some embodiments, the TR will be resistant to the process of gene conversion as described by Samulski et al., (1983) Cell 33:135. Gene conversion at the non-resolvable TR will restore the trs site, which will generate a resolvable TR and result in an increase in the frequency of monomeric replication products. Gene conversion results by homologous recombination between the resolvable TR and the altered TR.

One strategy to reduce gene conversion is to produce virus using a cell line (e.g., a mammalian cell line) that is defective for DNA repair, as known in the art, as these cell lines will be impaired in their ability to correct the mutations introduced into the viral template. Alternatively, templates that have a substantially reduced rate of gene conversion can be generated by introducing a region of non-homology into the non-resolvable TR. Non-homology in the region surrounding the trs element between the non-resolvable TR and the unaltered TR on the template will reduce or even substantially eliminate gene conversion.

Any suitable insertion or deletion may be introduced into the non-resolvable TR to generate a region of non-homology, as long as gene conversion is reduced or substantially eliminated. Strategies that employ deletions to create non-homology are preferred. It is further preferred that the deletion does not unduly impair replication and packaging of the template. In the case of a deletion, the same deletion may suffice to impair resolution of the trs site as well as to reduce gene conversion. In some embodiments, gene conversion can be reduced by insertions into the non-resolvable TR or, alternatively, into the A element between the RBE and the trs site. The insertion can be at least about 3, 4, 5, 6, 10, 15, 20 or 30 nucleotides or more nucleotides in length. There is no particular upper limit to the size of the inserted sequence, which may be as long as 50, 100, 200 or 500 nucleotides or longer, however, it is preferred that the insertion does not unduly impair replication and packaging of the template.

In some embodiments, the non-resolvable TR may be a naturally-occurring TR (or altered form thereof) that is non-resolvable under the conditions used. For example, the non-resolvable TR may not be recognized by the Rep proteins used to produce the vDNA from the template. To illustrate, the non-resolvable TR may be an autonomous parvovirus sequence that is not recognized by AAV Rep proteins. As a yet further alternative, the non-resolvable sequence may be any inverted repeat sequence that forms a hairpin structure and cannot be cleaved by the Rep proteins.

In other embodiments, a half-genome size template may be used to produce a parvovirus particle carrying a duplexed vDNA, produced from a half-genome sized template, as described in Hirata & Russell, (2000) J. Virology 74:4612, which describes packaging of paired monomers and transient RF intermediates when AAV genomes were reduced to less than half-size of the wtAAV genome (<2.5 kb). These investigators found that monomeric genomes were the preferred substrate for gene correction by homologous recombination, and that duplexed genomes functioned less well than did monomeric genomes in this assay. This report did not investigate or suggest the use of duplexed genomes as vectors for gene delivery.

In some embodiments, the template will be approximately one-half of the size of the vDNA that can be packaged by the parvovirus capsid. For example, for an AAV capsid, the template can be approximately one-half of the wt AAV genome in length, as described above. The template (as described above) is replicated to produce a duplexed vector genome (vDNA), which is capable of forming a double-stranded DNA under appropriate conditions. The duplexed molecule is substantially self-complementary so as to be capable of forming a double-stranded viral DNA (e.g., at least 90%, 95%, 98%, 99% nucleotide sequence complementarity, or more). Base-pairing between individual nucleotide bases or polynucleotide sequences is well-understood in the art. In some embodiments, the duplexed parvovirus viral DNA is essentially completely self-complementary (i.e., contains no or an insignificant number of mis-matched bases). In particular, it is preferred that the heterologous nucleotide sequence(s) (e.g., the sequences to be transcribed by the cell) are essentially completely self-complementary.

In general, the duplexed parvoviruses may contain non-complementarity to the extent that expression of the heterologous nucleotide sequence(s) from the duplexed parvovirus vector is more efficient than from a corresponding monomeric vector.

The duplexed parvoviruses provide the host cell with a double-stranded molecule that addresses the need for the host cell to convert the single-stranded rAAV vDNA into a double-stranded DNA.

The presence of any substantial regions of non-complementarity within the virion DNA, in particular, within the heterologous nucleotide sequence(s) will likely be recognized by the host cell, and will result in DNA repair mechanisms being recruited to correct the mismatched bases, thereby counteracting the advantageous characteristics of the duplexed parvovirus vectors, e.g., the vectors reduce or eliminate the need for the host cell to process the viral template.

The vectors disclosed herein, such as the adenovirus and AAV vectors, include a beta cell specific promoter operably linked to a nucleic acid transcribed to form the inhibitor RNA. The promoter can be, for example, an insulin, nkx6.1, nkx6.2, neuroD or pax4 promoter. In one specific non-limiting example, the vector is an AAV vector and the promoter is an insulin promoter.

### C. Chemical Compounds, Small Molecules and Caspase Inhibitors

Smad2 inhibitors include molecules that are identified from large libraries of both natural product or synthetic (or semi-synthetic) extracts or chemical libraries. Screening methods that detect decreases in smad2 protein activity are useful for identifying compounds from a variety of sources for activity. The initial screens may be performed using a diverse library of compounds, a variety of other compounds and compound libraries. Thus, molecules that bind smad2 protein, that inhibit the expression of smad2 protein, and molecules that inhibit the activity of smad2 protein can be identified. These small molecules can be identified from combinatorial libraries, natural product libraries, or other small molecule libraries. In addition, smad2 inhibitor can be identified as compounds from commercial sources, as well as commercially available analogs of identified inhibitors. The smad2 inhibitor can be tested, for example, in an assay to confirm it affects, for example, transcription, translation, or protein activity.

The precise source of test extracts or compounds is not critical to the identification of smad2 protein small molecule antagonists. Accordingly, smad2 protein inhibitors can be identified from virtually any number of chemical extracts or compounds. Examples of such extracts or compounds that can be smad2 protein inhibitors include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). smad2 inhibitors can be identified from synthetic compound libraries that are commercially available from a number of companies including Maybridge Chemical Co. (Trevillet, Cornwall, UK), Comgenex (Princeton, N. J.), Brandon Associates (Merrimack, N.H.), and Microsource (New Milford, Conn.). Smad2 protein inhibitors can be identified from a rare chemical library, such as the library that is available from Aldrich (Milwaukee, Wis.). Smad2 protein inhibitors can be identified in libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Natural and synthetically produced libraries and compounds are readily modified through conventional chemical, physical, and biochemical means.

Useful compounds that function as inhibitors can be found within numerous chemical classes, though typically they are organic compounds, including small organic compounds. Small organic compounds have a molecular weight of more than 50 yet less than about 2,500 daltons, such as less than about 750 daltons or less than about 350 daltons can be utilized in the methods disclosed herein. Exemplary classes include heterocycles, peptides, saccharides, steroids, and the like. The compounds may be modified to enhance efficacy, stability, pharmaceutical compatibility, and the like.

Compounds of use in the disclosed methods include those that block TGFβ signaling. See, for example, Lucaciu et al., Eur J Gastroenterol Hepatol. 2020 June; 32(6):669, PMID: 32282548; Wang et al., Eur J Med Chem. 2020 Apr. 1; 191:112154, PMID: 32092587; and Wang et al., Cell Metab. 2019 Mar. 5; 29(3):638-652.e5, PMID: 30581122, all incorporated herein by reference.

### Disruption of the Smad2 Gene

Methods and compositions are disclosed herein for altering the smad2 gene in beta cells in the endocrine pancreas. The methods and compositions described herein introduce one or more breaks near the site of the smad2 gene to decrease the production of functional smad2 protein, for example, in a pancreatic beta cell.

An exemplary nucleic acid encoding human smad2 is disclosed in NCBI Ref. Seq. No. NM_001003652.4, Feb. 20, 2021, incorporated herein by reference, and an exemplary protein sequence is disclosed in NCBI Ref. Seq. No. NP_001003652.1, Feb. 20, 2021, both incorporated herein by reference. Additional sequences are provided, for example, in RefSeq NM_00590, as available on Apr. 19, 2021. Other examples are provided herein. Exemplary results are provided in the Examples section.

A typical CRISPR system is composed of two components, a CRISPR-associated nuclease 9 (Cas9) and one or more guide RNAs (gRNAs), each of which contain a CRISPR RNA (crRNA) and a trans-activating CRISPR RNA (tracrRNA). Simple gene disruptions can be generated by cleavage of the target site, followed by alteration of nucleic acids, such as a deletion, and repair by the non-homologous-end-joining pathway (NHEJ). Target recognition by crRNAs occurs through complementary base pairing with target DNA, which directs cleavage of foreign sequences by means of Cas proteins. In some embodiments, DNA recognition by guide RNA and consequent cleavage by the endonuclease requires complementary base-pairing with a protospacer adjacent motif (PAM) (e.g. 5′-NGG-3′) and with a protospacer region in the target. (Jinek et. al., Science. 337:816-821, 2012). The PAM motif recognized by a Cas9 varies for different Cas9 proteins. Any Cas9 protein can be used in the systems and methods disclosed herein.

One Cas9 of use is from Streptococcus pyogenes as depicted in below
(SEQ ID NO: 12)
MDKKYSIGLDIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSLG
LTPNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKLINGIRDKQSG
KTILDFLKSDGFANRNFMQLIHDDSLTFKEDIQKAQVSGQGDSLHEHIANLAGSPAIKKGIL
QTVKVVDELVKVMGRHKPENIVIEMARENQTTQKGQKNSRERMKRIEEGIKELGSQILKE
HPVENTQLQNEKLYLYYLQNGRDMYVDQELDINRLSDYDVDHIVPQSFLKDDSIDNKVLT
RSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKVEKGKSKKLKS
VKELLGITIMERSSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKEVLD
ATLIHQSITGLYETRIDLSQLGGD

In other embodiments, the Streptococcus pyogenes Cas9 peptide can include one or more of the mutations described in the literature, including but not limited to the functional mutations described in: Fonfara et al., Nucleic Acids Res. 2014 February; 42(4):2577-90; Nishimasu et al. Cell. 2014 Feb. 27; 156(5):935-49; Jinek M et al. Science. 2012 Aug. 17; 337(6096):816-21; and Jinek et al. Science. 2014 Mar. 14; 343(6176). Thus, in some embodiments the systems and methods disclosed herein can be used with the wild type Cas9 protein having double-stranded nuclease activity, Cas9 mutants that act as single stranded nickases, or other mutants with modified nuclease activity.

The Cas9 includes a catalytically active nuclease domain. In some embodiments, the Cas9 nuclease includes an HNH-like endonuclease and a RuvC-like endonuclease. In some embodiments, to generate a double-stranded DNA break, the HNH-like endonuclease cleaves the DNA strand complementary to the gRNA, and the RuvC-like domain cleaves the non-complementary DNA strand. A Cas9 endonuclease can be guided to specific genomic targets using specific gRNA (see below).

In other embodiments of the systems and methods disclosed herein, a promoter, such as a beta cell specific promoter, for example, the insulin, nkx6.1, nkx6.2, neuroD, or pax4 promoter, is operably linked to the nucleic acid encoding Cas9. The beta cell specific promoter provides for cell specific expression of Cas9 in beta cells.

Exemplary beta cell promoters are disclosed above. An exemplary promoters of use are provided in SEQ ID NOs: 5-11. The promoter can include 1, 2, 3, 4, 5, 6, 7, 8, 9 or 10 substitutions in any one of SEQ ID NOs: 5-11, provided the promoter allows for expression in beta cells. The promoter can be at least 95%, at least 96%, at least 97%, at least 98% or at least 99% identical to one of SEQ ID NOs: 5-11, provided that the promoter allows for expression in beta cells. The promoter can be an insulin promoter. The promoter can be a Pdx-1, NKX6.1, NKX6.2, NeuroD or Pax-4 promoter. Other beta cell specific promoters are also of use.

Optionally, a nucleic acid molecule encoding a marker also can be operably linked to the beta cell specific promoter, such as the insulin promoter. Markers include, but are not limited to, enzymes and fluorescent proteins. In one specific non-limiting example, the marker is tdTomato fluorescent protein. In other embodiments, a nucleic acid molecule encoding a marker is not operably linked to the beta cell specific promoter, but is expressed from a different promoter or is not included in the construct.

As noted above, the Cas9 RNA guide system includes a mature crRNA that is base-paired to a trans-activating crRNA (tracrRNA), forming a two-RNA structure that directs Cas9 to the locus of a desired double-stranded (ds) break in target DNA, namely the smad2 gene. In some embodiments, the base-paired tracrRNA:crRNA combination is engineered as a single RNA chimera to produce a guide sequence (e.g., gRNA) which preserves the ability to direct sequence-specific Cas9 dsDNA cleavage (see Jinek et al., Science. 337:816-821, 2012). In some embodiments, the Cas9-guide sequence complex results in cleavage of one or both strands at a target sequence within the smad2 gene, such as in exons of the smad2 gene. Thus, the Cas9 endonuclease (Jinek et al., Science. 337:816-821, 2012; Mali et. al., Nat Methods. 2013 October; 10(10): 1028-1034) and the gRNA molecules are used sequence-specific target recognition, cleavage, and genome editing of the smad2 gene. In one embodiment, the cleavage site is at a specific nucleotide, such as, but not limited to the 16, 17, or 18^{th}, nucleotide of a 20 nucleotide target. In one non-limiting example, the cleavage site is at the 17^{th} nucleotide of a 20-nt target sequence. The cleavage can be a double stranded cleavage.

In some embodiments, the gRNA molecule is selected so that the target genomic targets bear a protospacer adjacent motif (PAM). In some embodiments, DNA recognition by guide RNA and consequent cleavage by the endonuclease requires the presence of a protospacer adjacent motif (PAM) (e.g., 5′-NGG-3′) in immediately after the target. The PAM is present in the targeted nucleic acid sequence but not in the crRNA that is produced to target it. In some embodiments, the proto-spacer adjacent motif (PAM) corresponds to 2 to 5 nucleotides starting immediately or in the vicinity of the proto-spacer at the leader distal end. The PAM motif also can be NNAGAA, NAG, NGGNG, AWG, CC, CC, CCN, TCN, or TTC.

In some embodiments, cleavage occurs at a site about 3 base-pairs upstream from the PAM. In some embodiments, the Cas9 nuclease cleaves a double stranded nucleic acid sequence.

In some embodiments, the guide sequence is selected to reduce the degree of secondary structure within the sequence. Secondary structure may be determined by any suitable polynucleotide folding algorithm. Some programs are based on calculating the minimal Gibbs free energy. An example of one such algorithm is mFold (Zuker and Stiegler, Nucleic Acids Res. 9 (1981), 133-148). Another example folding algorithm is the online webserver RNAfold, which uses the centroid structure prediction algorithm (see e.g., Gruber et al., 2008, Cell 106(1): 23-24; and Can and Church, 2009, Nature Biotechnology 27(12): 1151-62). Guide sequences can be designed using the MIT CRISPR design tool found at crispr.mit.edu, Harvard and University of Bergen CHOPCHOP web tool found at chopchop.cbu.uib.no, or the E-CRISP tool found at www.e-crisp.org/E-CRISP. Additional tools for designing tracrRNA and guide sequences are described in Naito et al., Bioinformatics. 2014 Nov. 20, and Ma et al. BioMed Research International, Volume 2013 (2013), Article ID 270805. The crRNA can be 18-48 nucleotides in length. The crRNA can be 18, 19, 20, 21, 22, 23, 24 or 25 nucleotides in length. In one example, the crRNA is 20 nucleotides in length. In additional embodiments, the tracrRNA is pre-optimized, and is 83 nucleotides in length, see, e.g., SEQ ID NO: 13.

(SEQ ID NO: 13 PMID: 31695072)
AGCAUAGCAAGUaUAAAUAAGGCUAGUCCGUUAUCAACUUGAAAAAGUG
GCACCGAGUCGGUGCUUU

In some embodiments, the human smad2 gene is targeted, and the crRNA is encoded by a nucleic acid sequence set forth as one of SEQ ID NOs: 14-20. In specific non-limiting examples, the crRNA is encoded by one of the DNA sequences below. The PAM, which is recognized by Cas9 is next to the target DNA sequence.

SMAD2 exon 2 crRNA:
(SEQ ID NO: 14)
AGCAGTCTCTTCACAACTGG
SMAD2 exon 3 crRNA:
(SEQ ID NO: 15)
CACCAAATACGATAGATCAG
SMAD2 exon 4 crRNA
(SEQ ID NO: 16)
TATCAGAGAGTTGAGACACC
SMAD2 exon 5 crRNA:
(SEQ ID NO: 17)
GAGTGAGTATAGTCATCCAG
SMAD2 exon 6 crRNA
(SEQ ID NO: 18)
TTGAATCAAAGTATGGACAC
SMAD2 exon 8 crRNA:
(SEQ ID NO: 19)
AAGCCATCTACAGTGAGTGA
SMAD2 exon 9 crRNA:
(SEQ ID NO: 20)
GCGCTTATACTACATAGGTG.

Additional sequences are provided in SEQ ID NOs: 24-26. These included two parts, crispr RNA (crRNA), a 17-20 nucleotide sequence complementary to the target DNA, and a tracr RNA, which serves as a binding scaffold for the saCas nuclease.

The system disclosed herein introduces double stranded DNA breaks at the smad2 gene, such that the smad2 target is cleaved by Cas9. This results in functional smad2 protein not being produced.

The system disclosed herein can include a promoter, such as, but not limited to, a U6 or H1 promoter operably linked to one or more nucleotide sequences transcribed to form one or more CRISPR-Cas RNAs.

The U6 promoter can include the following nucleic acid sequence:

GAGGGCCTATTTCCCATGATTCCTTCATATTTGCATATACGATACAAGG
CTGTTAGAGAGATAATTGGAATTAATTTGACTGTAAACACAAAGATATT
AGTACAAAATACGTGACGTAGAAAGTAATAATTTCTTGGGTAGTTTGCA
GTTTTAAAATTATGTTTTAAAATGGACTATCATATGCTTACCGTAACTT
GAAAGTATTTCGATTTCTTGGCTTTATATATCTTGTGGAAAGGACGAAA
CACC (SEQ ID NO: 21, see also GENBANK ® Accession
No. X07425.1, incorporated herein by reference).

Disclosed below is a nucleic acid transcribed to form a U6 gRNA sequence, wherein the tracrRNA is underlined. The tracer sequence includes seven thymidines for terminating RNA transcription. The small “g,” “ga,” and the second “g” border the SapIrev and SapI sites where the nucleic acid transcribed to form the gRNA is inserted.

(SEQ ID NO: 22)
GGCGCGCCGGATCCGAGGGCCTATTTCCCATGATTCCTTCATATTTGCA
TATACGATACAAGGCTGTTAGAGAGATAATTGGAATTAATTTGACTGTA
AACACAAAGATATTAGTACAAAATACGTGACGTAGAAAGTAATAATTTC
TTGGGTAGTTTGCAGTTTTAAAATTATGTTTTAAAATGGACTATCATAT
GCTTACCGTAACTTGAAAGTATTTCGATTTCTTGGCTTTATATATCTTG
AAATAGCAAGTTAAAATAAGGCTAGTCCGTTATCAACTTGAAAAAGTGG
CTGGAAAGGACGAAACACCgGAAGAGCgaGCTCTTCgGTTTTAGAGCTA
GACCGAGTCGGTGCTTTTTTTGGTACCGGCGCGCC

In one example, the tracrRNA is encoded by the nucleic acid sequence set forth as:

(SEQ ID NO: 23)
GTTTTAGAGCTAGAAATAGCAAGTTAAAATAAGGCTAGTCCGTTATCAA
CTTGAAAAAGTGGCACCGAGTCGGTGCTTTTTTT.

In some embodiments, more than one DNA break can be introduced by using more than one gRNA. For example, two gRNAs can be utilized, such that two breaks are achieved. When two or more gRNAs are used to position two or more cleavage events, in a target nucleic acid, it is contemplated that in an embodiment the two or more cleavage events may be made by the same or different Cas9 proteins. For example, when two gRNAs are used to position two double strand breaks, a single Cas9 nuclease may be used to create both double strand breaks.

In some embodiments, the disclosed methods include the use of one or more vectors comprising: a) a beta cell specific promoter operably linked to a nucleotide sequence encoding a Type II Cas9 nuclease, b) a U6 promoter operably linked to one or more nucleotide sequences transcribed to form one or more CRISPR-Cas guide RNAs that hybridize with the smad2 gene in a target cell, such as a human cell. Components (a) and (b) can be located on same or different vectors, whereby the one or more guide RNAs target the smad2 gene in the target cell and the Cas9 protein cleaves the smad2 gene. In specific non-limiting examples, the one or more vectors are viral vectors such as lentiviral vectors. In other non-limiting examples, the viral vectors are adenovirus vectors, adeno-associated virus vectors, or retroviral vectors.

Lentiviral vectors are retroviral vectors that are able to transduce or infect non-dividing cells and typically produce high viral titers. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. One non-limiting example of a lentiviral vector is the lentiCRISPRv2 vector (Adgene Plasmid #52961, see the addgene website, addgene.org/52961/).

Retroviral vectors of use in this embodiment also include murine leukemia virus (MuLV) vectors, gibbon ape leukemia virus (GaLV) vectors, Simian Immunodeficiency virus (SIV) vectors, human immuno deficiency virus (HIV) vectors, and combinations thereof (see, e.g., Buchscher et al., (1992) J. Virol. 66:2731-2739; Johanti et al., (1992) J. Virol. 66: 1635-1640; Sommnerfeit et al., (1990) Virol. 176:58-59; Wilson et al., (1998) J. Virol. 63:2374-2378; Miller et al., (1991) J. Virol. 65:2220-2224; PCT/US94/05700). The use of lentiviral vectors for the delivery of Cas9 and sgRNAs is disclosed in U.S. Published Patent Application No. US20150191744, which is incorporated herein by reference. Other vectors are use are disclosed above.

Methods are disclosed herein for altering expression of smad2 in a subject. The method included introducing into an endocrine cell, such as a human beta cell comprising a gene encoding smad2, an engineered, non-naturally occurring Clustered Regularly Interspaced Short Palindromic Repeats (CRISPR)-associated (Cas) system comprising one or more viral vectors, such as lentiviral vectors. The one or more viral vectors include a) an beta cells specific promoter (such as, but not limited to, the insulin promoter) operably linked to a nucleotide sequence encoding a Cas9 protein, and b) a U6 promoter operably linked to at least one nucleotide sequence transcribed to form a CRISPR-Cas guide RNA that hybridizes with the smad2 gene, wherein components (a) and (b) are located on same or different vectors of the system, whereby the guide RNA targets the smad2 gene and the Cas9 protein cleaves the smad2 gene in beta cells.

In some embodiments, the Cas9 protein is expressed in a recombinant cell, such as E. coli, and purified. The resulting purified Cas9 protein, along with an appropriate guide molecule specific for the target, is then introduced into a cell or organism where one or genomic sequences can be targeted. In some examples, the Cas9 protein and guide nucleic acid molecule (i.e., gRNA) are introduced as separate components into the target cell. In other examples, the purified Cas9 protein is complexed with the guide nucleic acid, and this ribonucleoprotein (RNP) complex is introduced into target cells (e.g., using transfection or injection). In some examples, the Cas9 protein and guide molecule are injected into the cell of interest. Once the Cas9 protein and guide nucleic acid molecule are in the cell, one or more genomic sequences can be targeted. Methods for introduction into the pancreatic duct are disclosed below.

### Methods of Treatment and Pharmaceutical Compositions

Methods are disclosed for treating T2DM in a subject. The subject can be any mammalian subject, including human and veterinary subjects. The subject can be an adult. The method can include selecting a subject of interest, such as a subject with T2DM.

The subject can also be administered metformin. The disclosed methods can also include having the subject make lifestyle modifications, such as increased physical activity, low fat diet, low sugar diet, and smoking cessation. In some embodiments, the subject can be administered an effective dose of one or more anti-diabetic agents (such as biguanides, thiazolidinediones, or incretins) and/or lipid lowering compounds (such as statins or fibrates).

In some examples, a subject with diabetes may be clinically diagnosed by a fasting plasma glucose (FPG) concentration of greater than or equal to 7.0 millimole per liter (mmol/L) (126 milligram per deciliter (mg/dL)), or a plasma glucose concentration of greater than or equal to 11.1 mmol/L (200 mg/dL) at about two hours after an oral glucose tolerance test (OGTT) with a 75 gram (g) load, or in a patient with classic symptoms of hyperglycemia or hyperglycemic crisis, a random plasma glucose concentration of greater than or equal to 11.1 mmol/L (200 mg/dL), or HbA1c levels of greater than or equal to 6.5%. Additional information can be found in Standards of Medical Care in Diabetes—2010 (American Diabetes Association, Diabetes Care 33:S11-61, 2010, incorporated herein by reference).

The disclosed pharmaceutical compositions including can be delivered to humans or other animals by any means, including orally, intravenously, intramuscularly, intraperitoneally, intranasally, intradermally, intrathecally, subcutaneously, via inhalation or via suppository. In one non-limiting example, the composition is administered into the pancreatic duct of a subject in vivo.

Appropriate doses depend on the subject being treated (e.g., human, non-human primate, or other mammal), age and general condition of the subject to be treated, the severity of the condition being treated, the agent selected, and the mode of administration, among other factors. An appropriate effective amount can be readily determined by one of skill in the art. Thus, a “therapeutically effective amount” will fall in a relatively broad range that can be determined through clinical trials. The method can include measuring an outcome, such as insulin production, improvement in a fasting plasma glucose tolerance test, or symptoms of the subject. In some examples, the therapeutically effective amount of the inhibitor is an amount that decreases the expression of smad2, thereby treating the T2DM. Decreasing expression of smad2 can be tissue or cell-type specific, for example, in some embodiments smad2 expression is decreased in beta cells in the endocrine pancreas. In some examples, administering the therapeutically effective amount of the inhibitor reduces plasma glucose levels or reduces glucose intolerance in the subject.

The dosage form of the pharmaceutical composition will be determined by the mode of administration chosen. For instance, in addition to injectable fluids, inhalation, oral and suppository formulations can be employed. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, PA, 21^{st} Edition (2005). For instance, parenteral formulations usually comprise injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

A therapeutically effective amount of the pharmaceutical composition can be administered in a single dose, twice daily, weekly, or in several doses, for example daily, or during a course of treatment. However, the therapeutically effective amount will be dependent on the subject being treated, the severity and type of the affliction, and the manner of administration of the therapeutic(s).

For in vivo injection of a vector, such as a lentiviral or AAV vector, i.e., injection directly to the subject, a therapeutically effective dose will be on the order of from about 10⁵ to 10¹⁶ of the virions, such as 10⁸ to 10¹⁴ virions. The dose depends on the efficiency of transduction, promoter strength, the stability of the message and the protein encoded thereby, and clinical factors. Effective dosages can be readily established by one of ordinary skill in the art through routine trials establishing dose response curves.

For delivery of virions, dosage treatment may be a single dose schedule or a multiple dose schedule to ultimately deliver the amount specified above. Moreover, the subject may be administered as many doses as appropriate. In some embodiments, the subject may be given, e.g., 10⁵ to 10¹⁶ AAV virions in a single dose, or two, four, five, six or more doses that collectively result in delivery of, e.g., 10⁵ to 10¹⁶ AAV virions. In some embodiments, the subject may be given, e.g., 10⁵ to 10¹⁶ lentiviral virions in a single dose, or two, four, five, six or more doses that collectively result in delivery of, e.g., 10⁵ to 10¹⁶ lentiviral virions. One of skill in the art can readily determine an appropriate number of doses to administer.

In some embodiments, the AAV or lentivirus is administered at a dose of about 1×10¹¹ to about 1×10¹⁴ viral particles (vp)/kg. In some examples, the AAV or lentivirus is administered at a dose of about 1×10¹² to about 8×10¹³ vp/kg. In other examples, the AAV or lentivirus is administered at a dose of about 1×10¹³ to about 6×10¹³ vp/kg. In specific non-limiting examples, the AAV or lentivirus is administered at a dose of at least about 1×10¹¹, at least about 5×10¹¹, at least about 1×10¹², at least about 5×10¹², at least about 1×10¹³, at least about 5×10¹³, or at least about 1×10¹⁴ vp/kg. In other non-limiting examples, the AAV or lentivirus is administered at a dose of no more than about 5×10¹¹, no more than about 1×10¹², no more than about 5×10¹², no more than about 1×10¹³, no more than about 5×10¹³, or no more than about 1×10¹⁴ vp/kg. In one non-limiting example, the AAV or lentivirus is administered at a dose of about 1×10¹² vp/kg. The AAV or lentivirus can be administered in a single dose, or in multiple doses (such as 2, 3, 4, 5, 6, 7, 8, 9 or 10 doses) as needed for the desired therapeutic results, such as the treatment of T2DM.

Pharmaceutical compositions include sufficient genetic material to produce reduce expression of smad2. In some embodiments, virions will be present in the subject compositions in an amount sufficient to provide a therapeutic effect, such as a change in insulin secretion, glucose tolerance, plasma glucose levels, and/or treatment of diabetes, such as T2DM, when given in one or more doses.

Virons, such as AAV or lentiviral virions can be provided as lyophilized preparations and diluted in a stabilizing compositions for immediate or future use. Alternatively, the virions can be provided immediately after production and stored for future use.

The pharmaceutical compositions can contain the vector, such as the AAV vector, lentiviral vector and/or virions, and a pharmaceutically acceptable excipient. Similarly, small molecules can also be included in a pharmaceutically acceptable excipient. In some embodiments, the excipients confer a protective effect on the virion such that loss of virions, as well as transduceability resulting from formulation procedures, packaging, storage, transport, and the like, is minimized. These excipient compositions are therefore considered “virion-stabilizing” in the sense that they provide higher virion titers and higher transduceability levels than their non-protected counterparts, as measured using standard assays, see, for example, Published U.S. Application No. 2012/0219528, incorporated herein by reference. These Compositions therefore demonstrate “enhanced transduceability levels” as compared to compositions lacking the particular excipients described herein, and are therefore more stable than their non-protected counterparts.

Exemplary excipients that can used to protect the virion from activity degradative conditions include, but are not limited to, detergents, proteins, e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, with molecular weights of 1500 to 6000 preferred, propylene glycols (PG), sugar alcohols, such as a carbohydrate, for example, sorbitol. The detergent, when present, can be an anionic, a cationic, a zwitterionic or a nonionic detergent. An exemplary detergent is a nonionic detergent. One suitable type of nonionic detergent is a sorbitan ester, e.g., polyoxyethylenesorbitan monolaurate (TWEEN®-20) polyoxyethylenesorbitan monopalmitate (TWEEN®-40), polyoxyethylenesorbitan monostearate (TWEEN®-60), polyoxyethylenesorbitan tristearate (TWEEN®-65), polyoxyethylenesorbitan monooleate (TWEEN®-80), polyoxyethylenesorbitan trioleate (TWEEN®-85), such as TWEEN®-20 and/or TWEEN®-80. These excipients are commercially available from a number of vendors, such as Sigma, St. Louis, Mo.

The amount of the various excipients present in any of the disclosed compositions varies and is readily determined by one of skill in the art. For example, a protein excipient, such as BSA, if present, will can be present at a concentration of between 1.0 weight (wt.) % to about 20 wt. %, such as 10 wt. %. If an amino acid such as glycine is used in the formulations, it can be present at a concentration of about 1 wt. % to about 5 wt. %. A carbohydrate, such as sorbitol, if present, can be present at a concentration of about 0.1 wt % to about 10 wt. %, such as between about 0.5 wt. % to about 15 wt. %, or about 1 wt. % to about 5 wt. %. If polyethylene glycol is present, it can generally be present on the order of about 2 wt. % to about 40 wt. %, such as about 10 wt. % top about 25 wt. %. If propylene glycol is used in the subject formulations, it will typically be present at a concentration of about 2 wt. % to about 60 wt. %, such as about 5 wt. % to about 30 wt. %. If a detergent such as a sorbitan ester (TWEEN®) is present, it can be present at a concentration of about 0.05 wt. % to about 5 wt. %, such as between about 0.1 wt. % and about 1 wt %, see U.S. Published Patent Application No. 2012/0219528, which is incorporated herein by reference. In one example, an aqueous virion-stabilizing formulation comprises a carbohydrate, such as sorbitol, at a concentration of between 0.1 wt. % to about 10 wt. %, such as between about 1 wt. % to about 5 wt. %, and a detergent, such as a sorbitan ester (TWEEN®) at a concentration of between about 0.05 wt. % and about 5 wt. %, such as between about 0.1 wt. % and about 1 wt. %. Virions are generally present in the composition in an amount sufficient to provide a therapeutic effect when given in one or more doses, as defined above.

The pharmaceutical compositions can include a contrast dye is administered in addition to a vector, such an adenoviral vector or lentivirus vector, including a beta cell-specific promoter operably lined to a nucleic acid molecule transcribed to form an inhibitory mRNA, or in addition to a small molecule or CRISPR-Cas9 system. The contrast dye can be a low-osmolar low-viscosity non-ionic dye, a low-viscosity high-osmolar dye, or a dissociable high-viscosity dye. In specific non-limiting examples, the dye is Iopromid, Ioglicinate, or Ioxaglinate. Thus, provided herein is a pharmaceutical composition including a) a vector, such as a AAV vector or a lentiviral vector, comprising a beta cell-specific promoter, such as an insulin, nkx6.1, nkx6.2, neurD, or pax4 promoter, operably linked to a nucleic acid molecule transcribed to form an inhibitor RNA specific for smad2; b) a buffer; and c) a contrast dye for endoscopic retrograde cholangiopancreatography. Also provided herein is a pharmaceutical composition including a) small molecule inhibitor of smad2; b) a buffer; and c) a contrast dye for endoscopic retrograde cholangiopancreatography. Further provided herein is a pharmaceutical composition including a) CRISPR/Cas9 system specific for smad2; b) a buffer; and c) a contrast dye for endoscopic retrograde cholangiopancreatography. The composition can be formulated for administration to the pancreatic duct

One exemplary method for intraductal administration is Endoscopic Retrograde Cholangiopancreatography (ERCP). ERCP is an endoscopic technique that involves the placement of a side-viewing instrument (generally either an endoscope or duodenoscope) within the descending duodenum. The procedure eliminates the need for invasive surgical procedures for administration to the pancreatic duct.

In an ERCP procedure, the patient will generally lie on their side on an examining table. The patient will then be given medication to help numb the back of the patient's throat, and a sedative to help the patient relax during the examination. The patient then swallows the endoscope. The thin, flexible endoscope is passed carefully through the alimentary canal of the patient. The physician guides the endoscope through the patient's esophagus, stomach, and the first part of the small intestine known as the duodenum. Because of the endoscope's relatively small diameter, most patients can tolerate the unusualness of having the endoscope advanced through the opening of their mouth.

The physician stops the advancement of the endoscope when the endoscope reaches the junction where the ducts of the biliary tree and pancreas open into the duodenum. This location is called the papilla of Vater, or also commonly referred to as the ampulla of Vater. The papilla of Vater is a small mound of tissue looking and acting similarly to a nipple. The papilla of Vater emits a substance known as bile into the small intestine, as well as pancreatic secretions that contain digestive enzymes. Bile is a combination of chemicals made in the liver and is necessary in the act of digestion. Bile is stored and concentrated in the gallbladder between meals. When digestive indicators stimulate the gallbladder, however, the gallbladder squeezes the bile through the common bile duct and subsequently through the papilla of Vater. The pancreas secretes enzymes in response to a meal, and the enzymes help digest carbohydrates, fats, and proteins.

The patient will be instructed (or manually maneuvered) to lie flat on their stomach once the endoscope reaches the papilla of Vater. For visualization or treatment specifically within the biliary tree, the distal end of the endoscope is positioned proximate the papilla of Vater. A catheter is then advanced through the endoscope until the distal tip of the catheter emerges from the opening at the endoscope's distal end. The distal end of the catheter is guided through the endoscope's orifice to the papilla of Vater (located between the sphincter of Oddi) and advanced beyond the common channel and into the common bile duct. In the case of pancreas-specific delivery of reagents, the pancreatic duct proper can be entered.

ERCP catheters can be constructed from Teflon, polyurethane and polyaminde. ERCP catheters also can also be constructed from resin comprised of nylon and PEBA (see U.S. Pat. No. 5,843,028), and can be constructed for use by a single operator (see U.S. Pat. No. 7,179,252). At times, a spring wire guide may be placed in the lumen of the catheter to assist in cannulation of the ducts. A stylet, used to stiffen the catheter, must first be removed prior to spring wire guide insertion. An inflatable balloon tip catheter may be used to prevent back flow out of the targeted ductal system.

A dual or multi-lumen ERCP catheter in which one lumen could be utilized to accommodate the spring wire guide or diagnostic or therapeutic device, and in which a second lumen could be utilized for contrast media and/or dye infusion and or for administration of a pharmaceutical composition of use in the disclosed methods. In some embodiments, a contrast dye is administered. The contrast dye can be a low-osmolar low-viscosity non-ionic dye, a low-viscosity high-osmolar dye, or a dissociable high-viscosity dye. In specific non-limiting examples, the dye is Iopromid, Ioglicinate, or Ioxaglinate. Endoscopes have been designed for the delivery of more than one liquid solution, such as a first liquid composition including a pharmaceutical composition, and a second liquid composition including dye, see U.S. Pat. No. 7,597,662, which is incorporated herein by reference. Thus, the pharmaceutical composition can be delivered in the same or separate liquid compositions. Methods and devices for using biliary catheters for accessing the biliary tree for ERCP procedures are disclosed in U.S. Pat. Nos. 5,843,028, 5,397,302 5,320,602, which are incorporated by reference herein.

In additional examples, the vector is administered using a viral infusion technique into a pancreatic duct. Suitable methods are disclosed, for example, in Guo et al. Laboratory Invest. 93: 1241-1253, 2013, incorporated by reference herein.

In some examples, treating diabetes includes one or more of increasing glucose tolerance, decreasing insulin resistance (for example, decreasing plasma glucose levels, decreasing plasma insulin levels, or a combination thereof), decreasing serum triglycerides, decreasing free fatty acid levels, and decreasing HbA1c levels in the subject. In some embodiments, the disclosed methods include measuring glucose tolerance, insulin resistance, plasma glucose levels, plasma insulin levels, serum triglycerides, free fatty acids, and/or HbA1c levels in a subject. The method can include measuring beta cell function.

In other embodiments, the disclosed methods include comparing one or more indicator of diabetes (such as glucose tolerance, triglyceride levels, free fatty acid levels, or HbA1c levels) to a control, wherein an increase or decrease in the particular indicator relative to the control (as discussed above) indicates effective treatment of diabetes. The control can be any suitable control against which to compare the indicator of diabetes in a subject. In some embodiments, the control is a sample obtained from a healthy subject (such as a subject without diabetes). In some embodiments, the control is a historical control or standard reference value or range of values (such as a previously tested control sample, such as a group of subjects with diabetes, or group of samples from subjects that do not have diabetes). In further examples, the control is a reference value, such as a standard value obtained from a population of normal individuals that is used by those of skill in the art. Similar to a control population, the value of the sample from the subject can be compared to the mean reference value or to a range of reference values (such as the high and low values in the reference group or the 95% confidence interval). In other examples, the control is the subject (or group of subjects) treated with placebo compared to the same subject (or group of subjects) treated with the therapeutic compound in a cross-over study. In further examples, the control is the subject (or group of subjects) prior to treatment.

### EXAMPLES

Understanding signaling pathways that regulate the β-cell function to produce, store, and release insulin, and pathways that control β-cell proliferation can lead to the identification of new treatments for diabetes. TGF-β signaling is involved in regulating a broad range of cellular functions and physiological processes in pancreatic β-cells. The TGF-β signaling pathway involves direct intracellular mediators including smad2 and smad3. As disclosed herein, a role of TGF-β/smad2 signaling was demonstrated in regulating mature β-cell proliferation and function using β-cell-specific smad2 null mutant mice. Smad2 null mutant mice exhibited improved glucose tolerance and increased insulin secretion. Also, smad2-deficient mice had an increased β-cell proliferation and β-cell mass. Similarly, smad2 knockout improved glucose tolerance, insulin secretion and insulin sensitivity in a diet-induced dysglycemic mouse model. Additionally, ex vivo analysis of smad2-deficient islets showed an increased glucose stimulated insulin secretion and an upregulation of insulin synthetic and insulin secretory genes.

### Example 1

### Generation of β-Cell-Specific Smad2 Knockout (Smad2-βKO) Mice

To study the role of smad2 in pancreatic β-cells, mice carrying conditional smad2^{fx/fx} alleles (Ju et al., Mol Cell Biol. 2006; 26(2):654-67) were crossed with ins1^{cre} transgenic mice, expressing cre-recombinase in β-cells under control of the ins1 promoter (FIG. 1A) (Thorens et al., Diabetologia. 2015; 58(3):558-65.). Deletion of smad2 protein was confirmed by Western blot analysis and immunohistochemistry (FIGS. 1B & 1C). A significant decrease was seen in the amount of smad2 protein in smad2-,6KO mouse islets. Next, the expression of smad2 mRNA was examined in isolated smad2-βKO (ins1^{cre};smad2^{fx/fx}) islets by real time-PCR (FIG. 1D). Some smad2 mRNA expression was detected in smad2-βKO islets, likely due to the presence of endothelial cells, pericytes and other non-β endocrine cells (alpha, gamma, and delta cells), which are all ins1^{cre} negative.

### Example 2

### Enhanced Glucose Tolerance in Smad2-βKO Mice with Improved GSIS in Smad2-βKO Mice and Isolated Islets

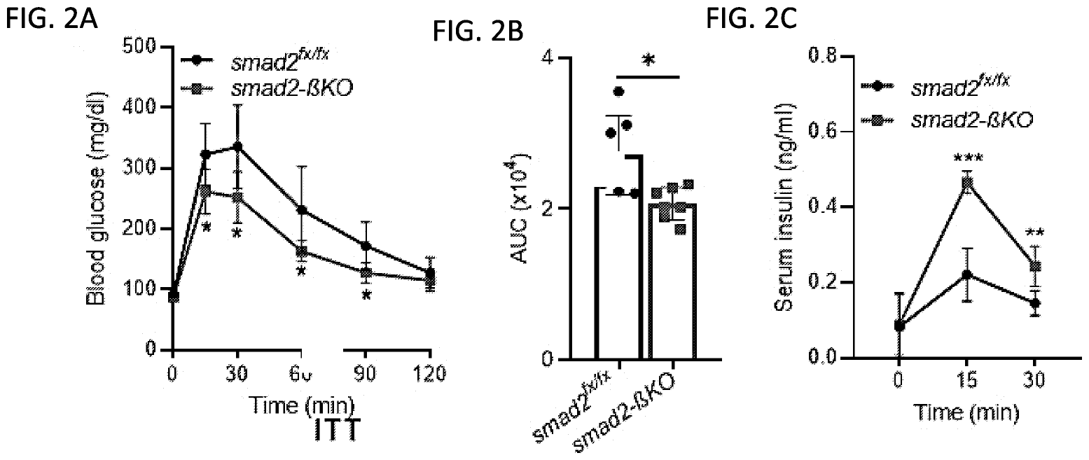

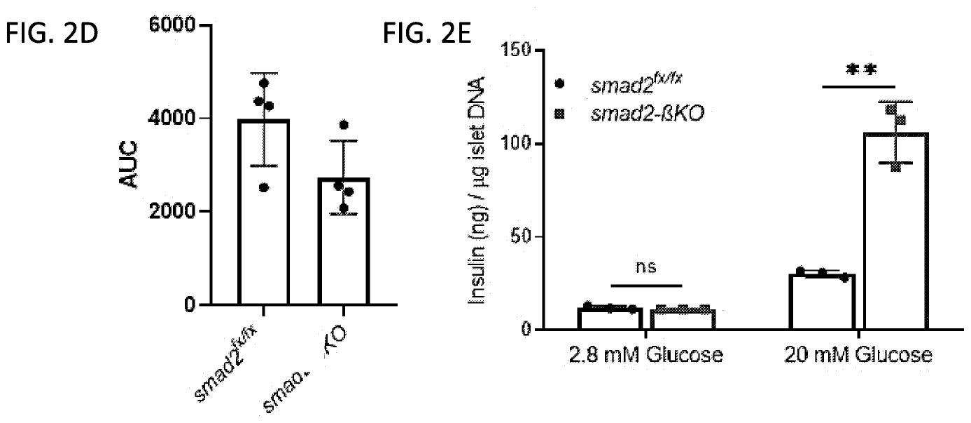

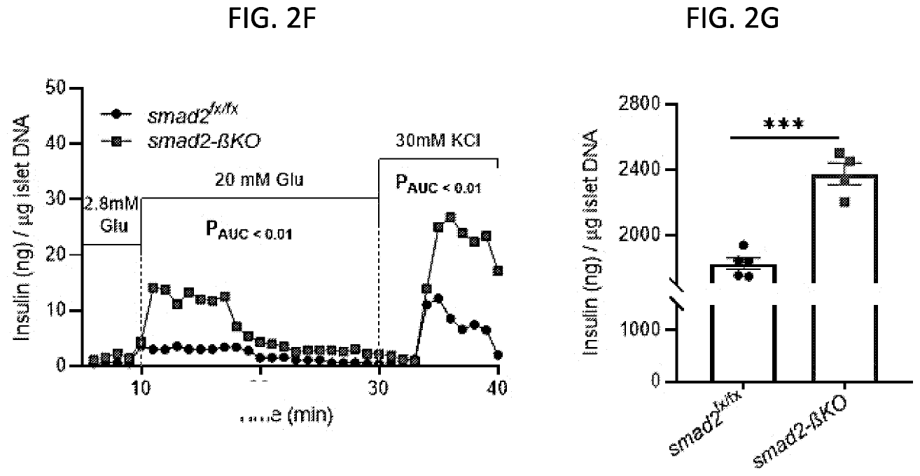

Smad2-βKO mice were indistinguishable in appearance from their cre-negative control littermates. They are viable and fertile with bodyweights comparable to their littermates for up to 1 year of observation. To examine the effect of the absence of smad2 on glucose homeostasis in vivo, intraperitoneal glucose tolerance test (IPGTT), GSIS, and intraperitoneal insulin tolerance testing (IPITT) were performed. Smad2-βKO mice showed improved glucose tolerance compared to littermate controls (FIGS. 2A & 2B). Also, smad2-βKO mice had a significant increase in the GSIS when compared to control mice (FIG. 2C). Sensitivity to insulin measured by IPITT was not significantly affected in smad2-βKO mice (FIG. 2D), which suggests that increased insulin secretion is the primary cause for improved glucose tolerance noticed in smad2-βKO mice.

Glucose stimulated insulin release was then studied in smad2-βKO isolated islets using a static GSIS assay (FIG. 2E), and an islet perifusion assay was used to assess dynamic insulin release (FIG. 2F). Smad2-βKO islets showed enhanced insulin release in response to high glucose concentration (20 mM) in the static GSIS (FIG. 2E). Similarly, in the dynamic perifusion study, there was a significant increase in insulin secretion in response to a high glucose concentration, with a robust burst of insulin release in response to KCl (FIG. 2F). To determine whether the increased insulin secretion correlates with greater insulin synthesis within the islets, the insulin content of islets isolated from smad2-βKO mice was assessed. Insulin content was significantly increased in smad2-βKO islets (FIG. 2G). These data suggest that smad2 may have an inhibitory effect on insulin synthesis, and on insulin secretion in response to glucose, both in vivo and ex vivo.

### Example 3

### Loss of Smad2 in β-Cells Enhances Expression of the Insulin Gene and Increases β-Cell Proliferation and β-Cell Mass

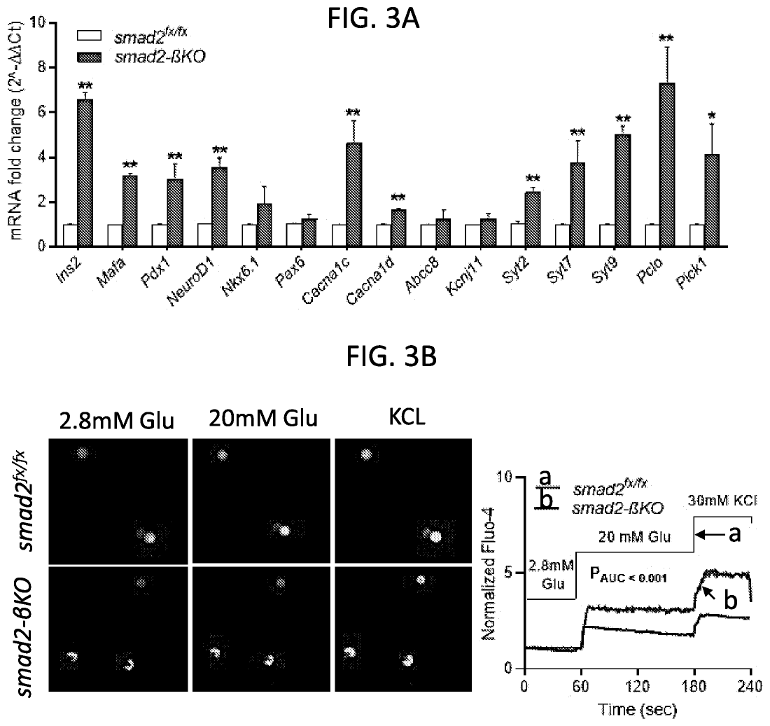

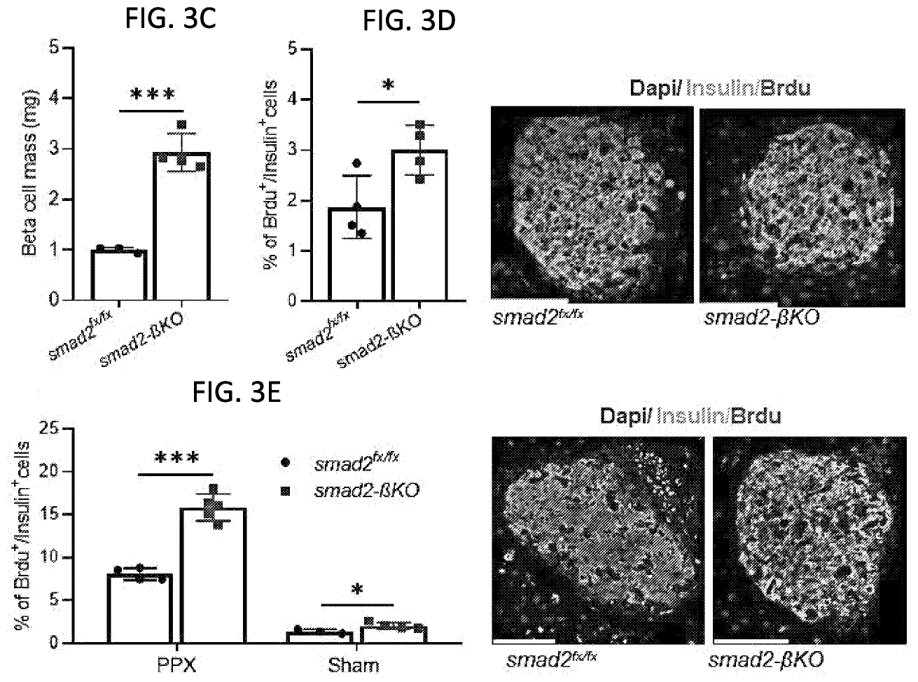

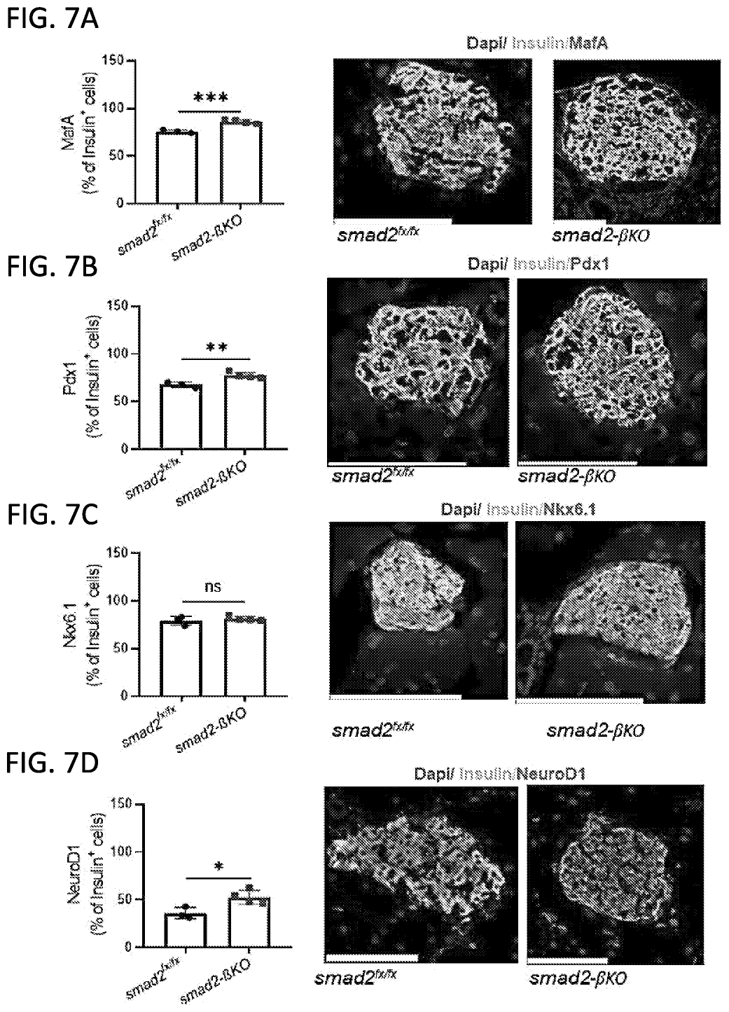

The role of smad2 in insulin synthesis, and its role in the expression of transcription factors that are crucial for R-cell function and proliferation, were investigated. Islets isolated from smad2-βKO mice had significantly higher expression of insulin2 mRNA compared to their wild-type littermate controls (FIG. 3A). Similarly, factors that directly regulate insulin gene expression (El Gohary et al., Dev Biol. 2013; 378(2):83-93), including MafA, Pdx1, and NeuroD1 are upregulated in smad2-βKO islets compared to controls (FIG. 3A). In keeping with the RT-PCR data, immunostaining of pancreas sections showed increased percentages of MafA⁺/Insulin⁺, Pdx1⁺/Insulin⁺ and NeuroD1⁺/Insulin⁺ cells in smad2-βKO (FIGS. 7A, 7B, 7D). Nkx6.1, a transcription factor required for maintaining β-cells in their differentiated state (Taylor et al., Cell Rep. 2013; 4(6):1262-75), and Pax 6, a transcription factor required for β-cell differentiation and optimal β-cell function (Gosman et al., Mol Endocrinol. 2012; 26(4):696-709), were similar in the smad2-βKO islets and the controls (FIG. 3A & FIG. 7C).

To study the effect of smad2 loss on β-cell proliferation, mice were treated with BrdU in the drinking water for one week to label proliferating β-cells. Quantification of BrdU⁺/insulin⁺ percentages showed a 1.6-fold increase in baseline β-cell proliferation in the knockout mice compared to controls (FIG. 3C). In addition, smad2-βKO mice had a higher β-cell mass compared to controls (FIG. 3D).

To further confirm that smad2 loss increases β-cell proliferation, since β-cell proliferation is low at baseline, 60% partial pancreatectomy (PPX) was used, a model for workload induced β-cell proliferation. After PPX, which increased β-cell proliferation 4-fold normally, there was a further 2.6-fold increase in the number of BrdU⁺ cells in smad2-βKO compared to controls (FIG. 3E). These data show that the overall suppression of TGF-β/smad2 signaling increases insulin synthesis, upregulates transcription factors involved in insulin gene expression, and induces β-cell proliferation.

Given the enhanced insulin secretion observed in smad2-βKO mice and their isolated islets, the effect of smad2 loss on the expression of potassium and calcium channels known to be involved in GSIS (Ashcroft et al., Nature. 1984; 312(5993):446-8; Straub and Sharp, Diabetes Metab Res Rev. 2002; 18(6):451-63) was assessed. The mRNA expression levels of the ATP-sensitive potassium (K-ATP) channel subunits (Kcnj11 and Abcc8) was similar in the smad2-βKO islets and controls. However, the mRNA expression of: 1) Cacna1c and Cacna1d (voltage-gated calcium channel subunits alpha1C and alpha1D), 2) Synaptotagmin-2, -7 and-9 and Piccolo (calcium sensor genes that facilitate calcium induced exocytosis (El Gohary et al., Dev Biol. 2013; 378(2):83-93; Gustavsson et al., Proc Natl Acad Sci USA. 2008; 105(10):3992-7; How et al., Vitam Horm. 2009; 80:473-506)) and 3) PICK1 (a PDZ domain-containing peripheral membrane protein that regulates the trafficking of insulin granules (Cao et al., PLoS Biol. 2013; 11(4):e1001541)), were all higher in smad2-βKO islets compared to controls (FIG. 3A).

Furthermore, the calcium flux was imaged in dispersed islet cells using the calcium indicator Fluo-4; individual cells were imaged during the stimulation by low glucose (2.8 mM), high glucose (20 mM) and KCl (20 mM). Smad2-βKO islet cells showed significantly increased Fluo-4 fluorescence in response to high glucose and KCl compared to control islets, indicating increased calcium influx (FIG. 3B). These data show that the suppression TGF-3/smad2 signaling enhances GSIS by increasing calcium-mediated insulin granule exocytosis.

### Example 4

### Deletion of Smad2 in β-Cells Mitigates HFD-Induced Glucose Intolerance and Improves Insulin Secretion In Vivo and in Isolated Islets

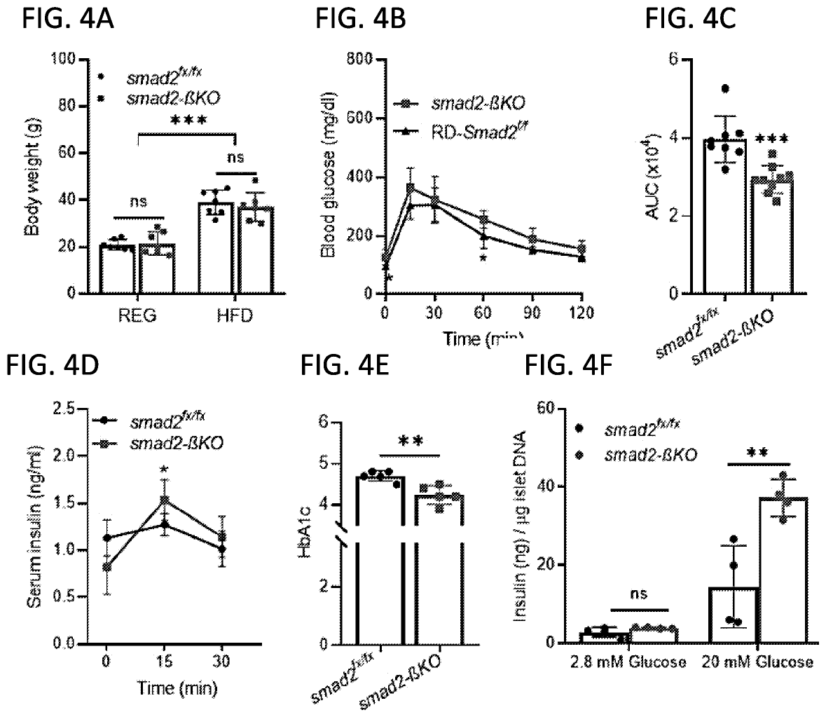

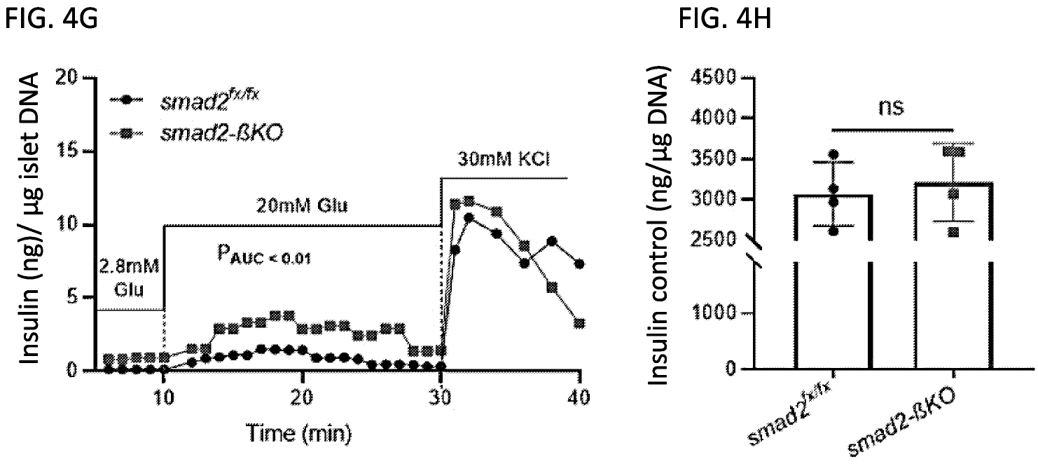

A mouse model was used in the studies disclosed herein that replicates obesity-induced dysglycemia in humans. HFD-induced obesity in C57BL/6J mice mirrors the human metabolic derangements of obesity (Eizirik et al., Proc Natl Acad Sci USA. 1994; 91(20):9253-6; Collins et al., Physiol Behav. 2004; 81(2):243-8; Mosser et al., Am J Physiol Endocrinol Metab. 2015; 308(7):E573-82). To investigate the potential involvement of TGFβ/smad signaling in HFD-induced hyperglycemia, 6-week old smad2-βKO mice and their littermate controls were put on 60% HFD or normal chow for 12 weeks. HFD-fed mice had significantly increased weight gain compared to controls, but no difference in body weight between smad2-βKO and littermates in either feeding regimens (FIG. 4A). After 12 weeks of HFD, HFD-smad2-βKO mice showed improved glucose tolerance compared to HFD-controls (FIG. 4B & 4C). In vivo GSIS done at the same time-point revealed a significant increase in the insulin secretion at 15 min in smad2-βKO mice compared to controls (FIG. 4D). It was also observed that HFD-smad2-βKO mice had lower fasting insulin levels with a trend towards significance (p=0.057) compared to HFD-controls (FIG. 4D), possibly indicating an improvement in the abnormal increase in endogenous hepatic glucose production reported in this model (Gastaldelli et al. (2000) Diabetes 49(8):1367-73). Furthermore, to investigate the effect of R-cell specific smad2 loss on long-term glucose homeostasis glycated hemoglobin (HbA1c) was measured. The HFD-smad2-βKO mice exhibited a slight but significant decrease in HbA1c compared to HFD-controls (FIG. 4H).

Next, islets isolated from smad2-βKO mice and their littermate controls were analyzed after 12 weeks of HFD. In keeping with the in vivo data, islets isolated from smad2-βKO mice showed enhanced insulin release in response to high glucose (20 mM) in both static GSIS (FIG. 4F) and dynamic perifusion studies (FIG. 4G), but no difference was observed between the two groups in insulin release after KCl stimulation (FIG. 4G). There was no difference observed for insulin content in islets isolated from HFD-smad2-βKO mice and HFD-controls (FIG. 4H).

### Example 5

### Improved Insulin Sensitivity Markers in the Liver and Other Peripheral Tissues in Smad2-βKO Mice after HFD Feeding

By IPITT, HFD-smad2-βKO mice had improved insulin sensitivity compared to HFD-controls (FIG. 5A). In support of these ITT results, calculation of the homeostasis model assessment of insulin resistance (HOMA-IR) index, a surrogate marker of insulin resistance (Fraulob et al., J Clin Biochem Nutr. 2010; 46(3):212-23), showed that HFD-smad2-βKO mice had a significantly lower HOMA-IR index than HFD-controls (FIG. 5B). To further investigate the improved insulin sensitivity in HFD-smad2-βKO, the protein expression levels of phosphorylated Akt (p-Akt), a key mediator of insulin-receptor signaling (Czech, Nat Med. 2017; 23(7):804-14), in the liver, skeletal muscle (quadriceps) and white adipose tissue (gonadal fat pad) was examined after 12 weeks of HFD. Immunoblotting showed an increased p-Akt/total Akt ratio in smad2-βKO when compared with controls (FIG. 5C).

The effect of smad2-βKO was also studied on hepatic steatosis in HFD mice (Matsuzaka and Shimano, J Diabetes Investig. 2011; 2(3):170-5). Oil-red-O staining of liver tissue was used to quantify the hepatocyte lipid-droplet accumulation (FIG. 5D), which showed a significantly lower percent area of liver tissue consisting of lipid droplets in HFD-smad2-βKO mice compared to HFD-controls. This finding indicates that smad2-βKO improves the fatty liver in HFD mice. The reported link between hepatic steatosis and hepatic insulin resistance (Jornayvaz et al., Proc Natl Acad Sci USA. 2011; 108(14):5748-52) further supports the finding of improved hepatic insulin sensitivity in HFD-smad2-βKO.

### Example 6

### Loss of Smad2 in β-Cells Decreases ER Stress, Increases β-Cell Proliferation and Mass in HFD-Fed Mice

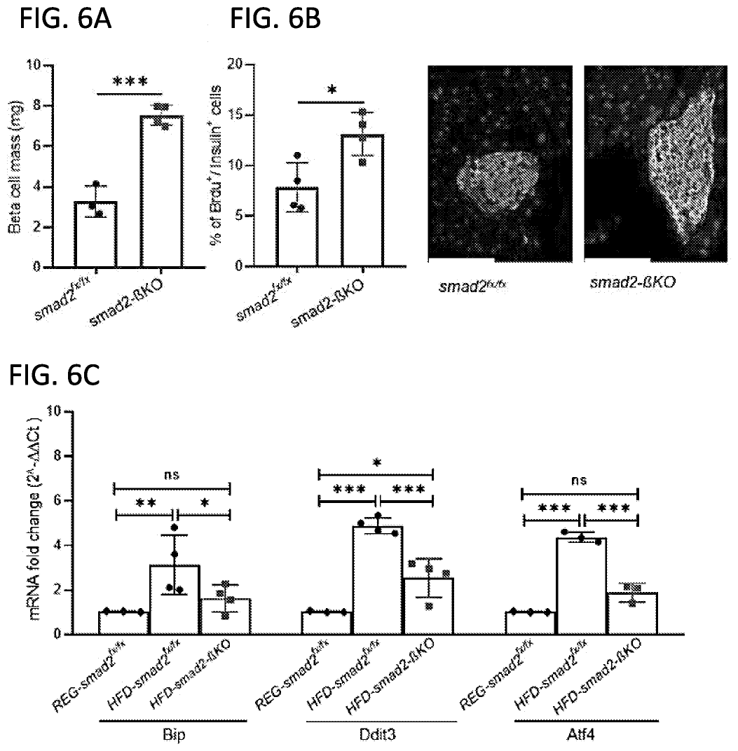

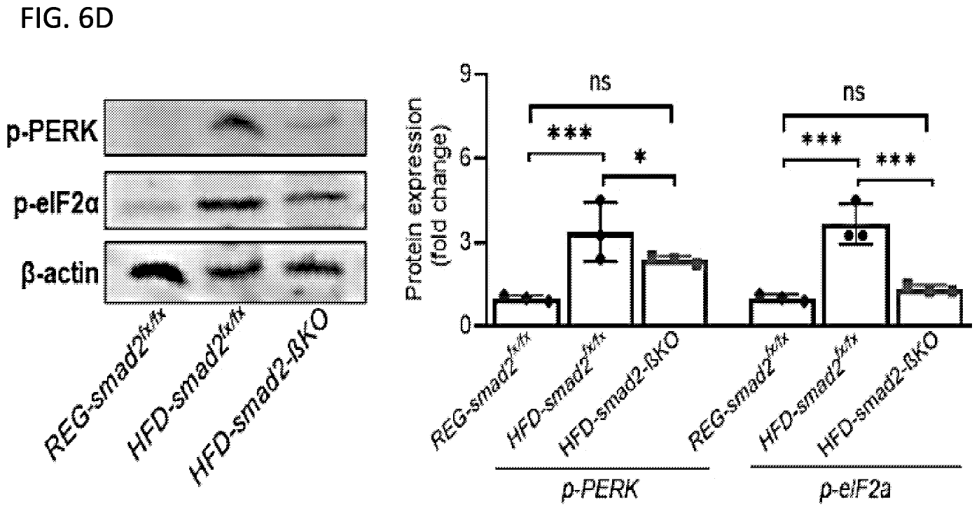

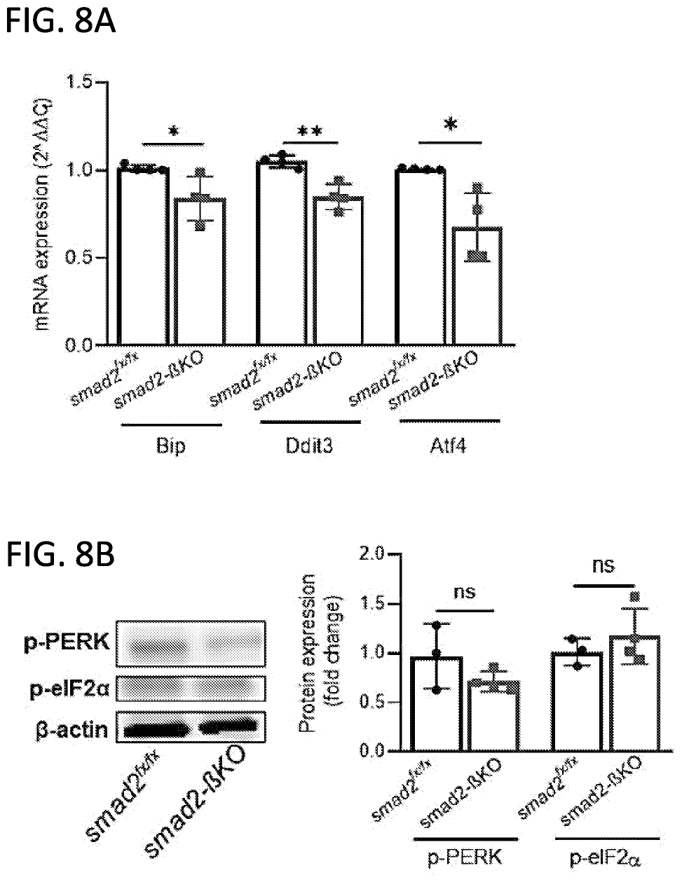

To investigate the possible mechanisms behind the improved adaptation of smad2-βKO mice to diet-induced metabolic stress, first, the changes in R-cell proliferation were investigated using BrdU incorporation after 12 weeks of HFD. R-cell proliferation in HFD-smad2-βKO mice was 1.6-fold higher compared to HFD-controls (FIG. 6A). In addition, there was a significant increase in the calculated β-cell mass of HFD-smad2-βKO mice compared to HFD-controls (FIG. 6B). Since β-cell mass expansion is well established in long-term HFD studies, with later occurrence of functional failure and ER stress (Mosser et al., Am J Physiol Endocrinol Metab. 2015; 308(7):E573-82; Stamateris et al., Am J Physiol Endocrinol Metab. 2013; 305(1):E149-59; Gupta et al., J Biol Chem. 2017; 292(30):12449-59), the effect of the absence of β-cell smad2 on diet-induced ER stress was investigated. The expression levels of various ER stress markers in isolated islets were assessed by qPCR (for BiP/Grp78, Chop (Ddit3) and Atf4), and protein blotting (for phosphorylated PERK and elF2α). Smad2-βKO on regular chow showed decreased BiP, Ddit3 and Atf4 gene expression in their islets, without significant change in the protein levels of p-PERK or p-elF2α when compared to regular chow controls (FIGS. 8A & 8B). However, islets isolated from HFD-smad2-βKO mice showed a significant decrease in the expression of ER stress markers compared to HFD-controls, but there was still an increase in ER stress markers in HFD-smad2-βKO islets when compared to islets from mice on regular chow (FIGS. 6C & 6D).

Thus, it was demonstrated that loss of smad2 in β-cells improves glucose tolerance and enhances insulin secretion, which reflects the improvement in β-cell function and increased β-cell mass observed in this model. In the experiments disclosed herein, loss of smad2 led to improved (3-cell function demonstrated by increased GSIS in vivo (FIG. 2C) and in isolated islets (FIGS. 2E & 2F). Moreover, smad2 knockout in β-cells led to upregulation of the insulin gene, insulin transcription factors, genes involved in insulin secretion and exocytosis (FIG. 3A), and increased insulin content (FIG. 2G).

Another study appeared contradictory to the findings presented herein, where Rip-cre/smad2^{fx/fx} mice showed impaired glucose tolerance, impaired insulin secretion, and islet hyperplasia (Nomura et al., Diabetologia. 2014; 57(1):157-66). In the mouse model used in that study, hGH is synthesized and secreted from the Rip-cre islets, causing cre-independent alterations in β-cell function (Brouwers et al., Cell Metab. 2014; 20(6):979-90). Also, several reports have shown that Rip-cre mice alone exhibit glucose intolerance (Lee et al., J Biol Chem. 2006; 281(5):2649-53; Pomplun et al., Horm Metab Res. 2007; 39(5):336-40). The inslere mouse model used in our study has been shown to induce effective and selective recombination of floxed genes in β-cells, without recombination in the central nervous system (Thorens et al., Diabetologia. 2015; 58(3):558-6), as opposed to Rip-cre mice, which is known to express cre in the hypothalamus (Collins et al., Physiol Behav. 2004; 81(2):243-8; Gannon et al., Genesis. 2000; 26(2):139-42), which may affect the weight and the feeding behavior of these transgenic mice (Nomura et al., Diabetologia. 2014; 57(1):157-66). These issues all raised concerns about the cell-type specificity and the interpretation of the phenotypes found in Rip-cre mice (Estall et al., Endocrinology. 2015; 156(7):2365-7).

The present work shows that loss of β-cell-specific smad2 caused an increase in β-cell mass and proliferation (FIGS. 3C & 3D). It was also demonstrated that PPX in smad2-βKO mice led to robust β-cell proliferation, which suggests that TGF-β/smad2 signaling has a significant effect on proliferation when there is an increase in the workload demand. The effect of smad2 deletion in a HFD-fed C57BL/6 mouse model that mirrors the metabolic derangements that occur in humans with obesity (Collins et al., Physiol Behav. 2004; 81(2):243-8; Mosser et al., Am J Physiol Endocrinol Metab. 2015; 308(7):E573-82). In line with the data obtained in non-obese mice, loss of smad2 in HFD-induced obese mice improved glucose tolerance, insulin secretion and insulin sensitivity, and increased beta-cell mass. Also, the improvement in glucose homeostasis parameters in the HFD-smad2-βKO mice was associated with a significant reduction in the fatty liver changes reported to happen in HFD-fed mice. Additionally, improved β-cell function observed in smad2-βKO obese mice was associated with decreased ER stress markers in the isolated islets (FIGS. 6C & 6D), suggesting that blocking TGF-3/smad signaling is protective against diet-induced ER stress in β-cells. Thus, evidence was provided that smad2 plays a vital role in regulating β-cell function and proliferation.

### Example 7

### The Acute Deletion of Smad2 in β-Cells (Acute Smad2-βKO) Mitigates High-Fat Diet-Induced (HFD-Induced) Glucose Intolerance Showing the Therapeutic Potential of this Strategy

HFD-has been used to induce obesity in C57BL/6J mice, and it mirrors the human metabolic derangements of obesity. To investigate the potential involvement of TGFβ/smad signaling in HFD-induced dysglycemia, 6-week-old mice were treated with a cre inducible smad2-βKO insertion with either a 60% HFD or standard chow for 12 weeks.

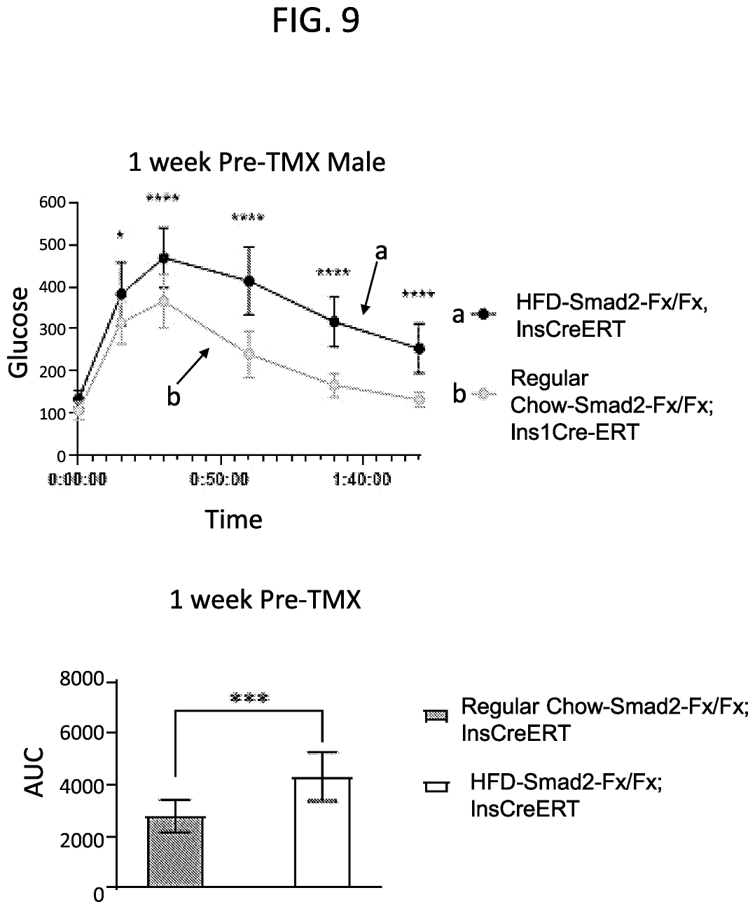

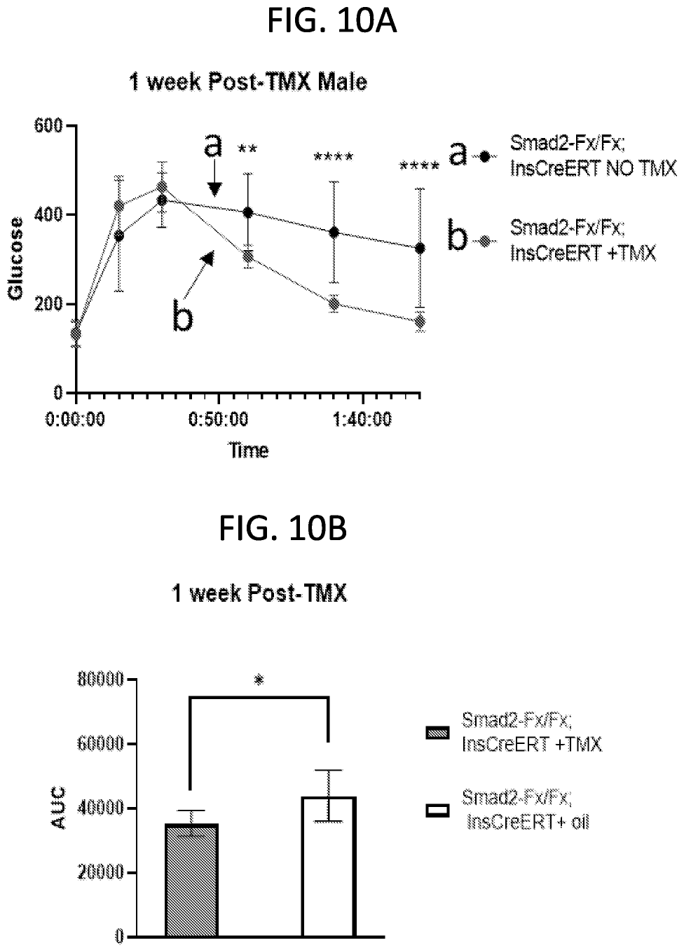

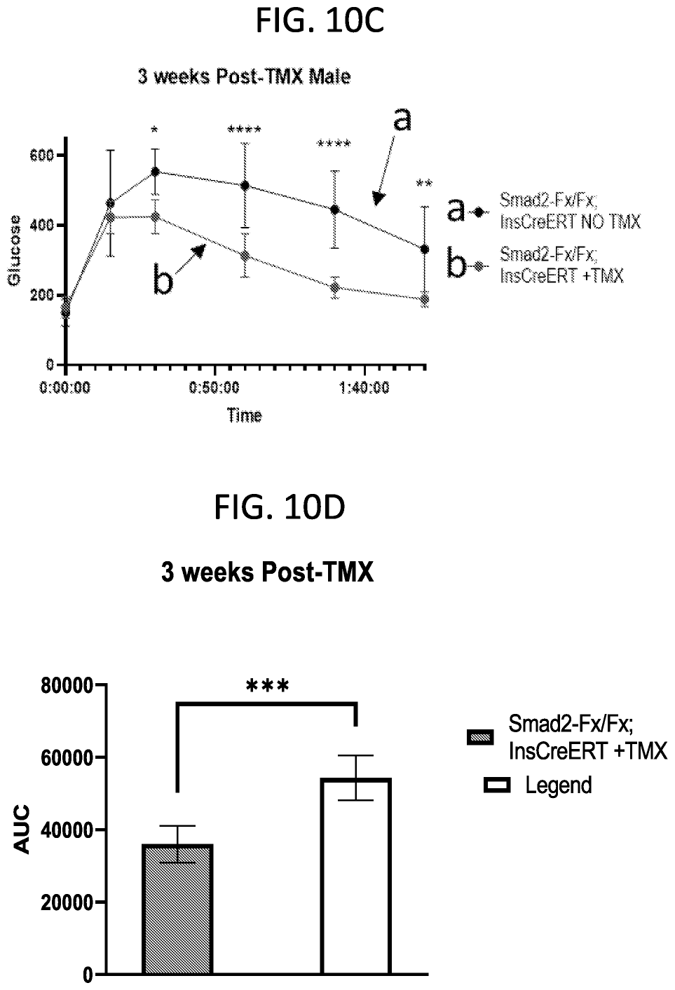

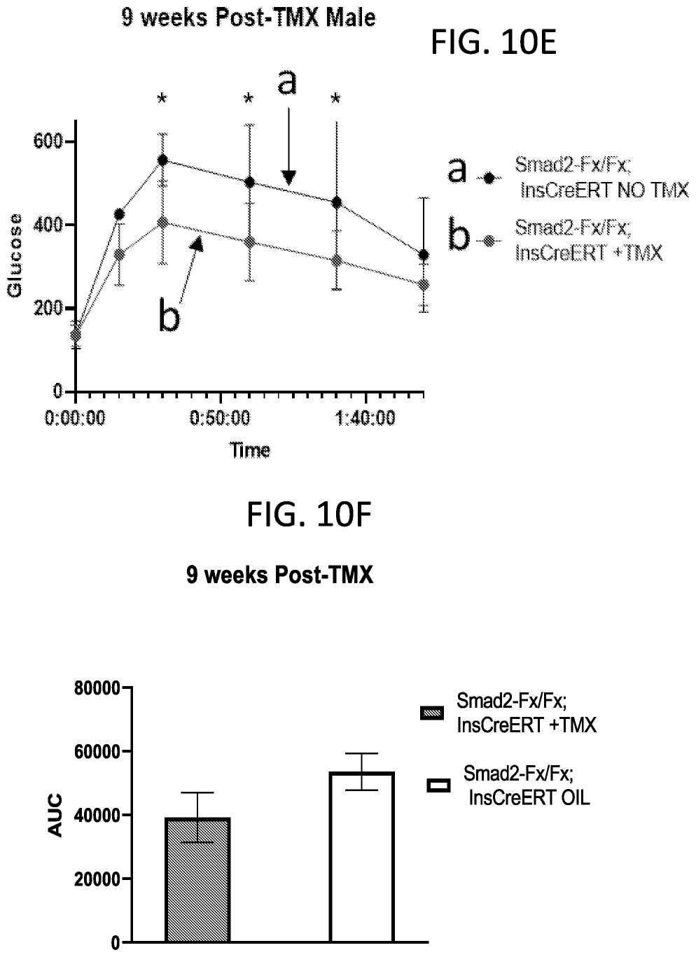

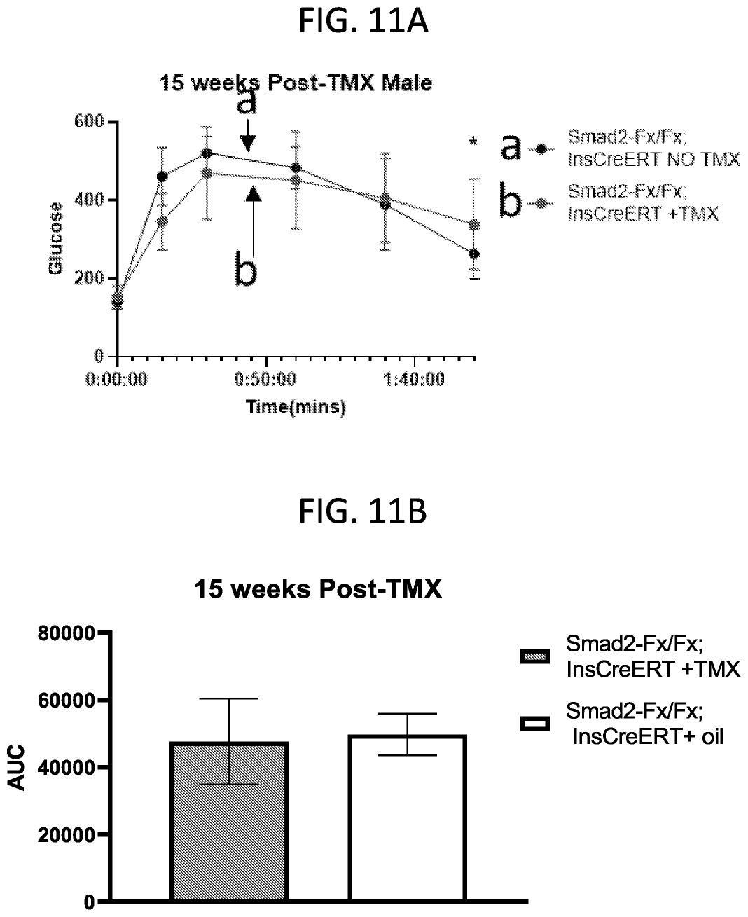

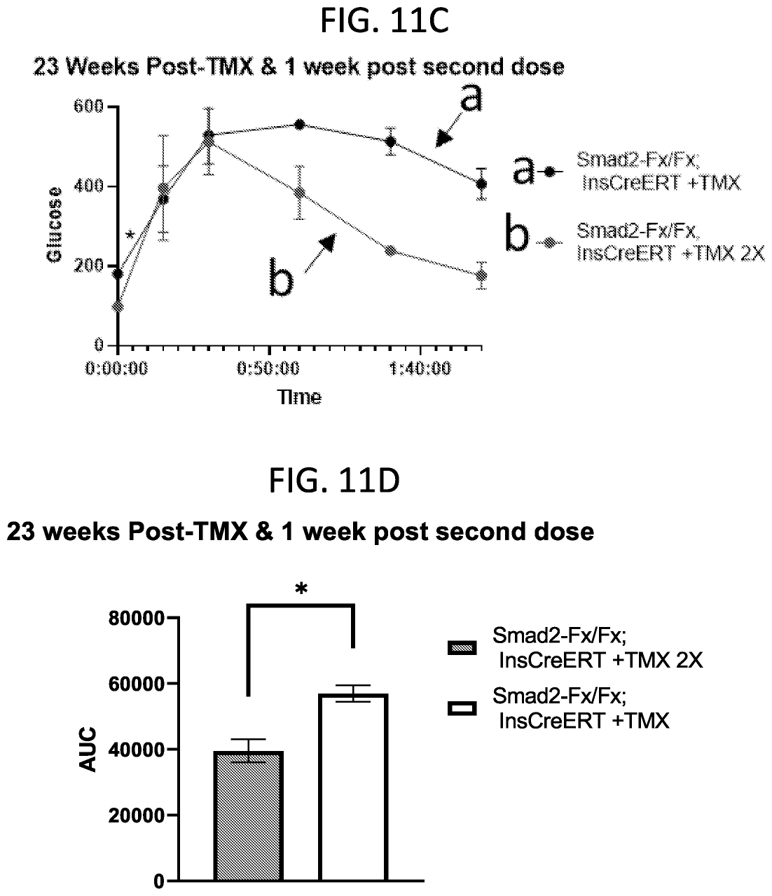

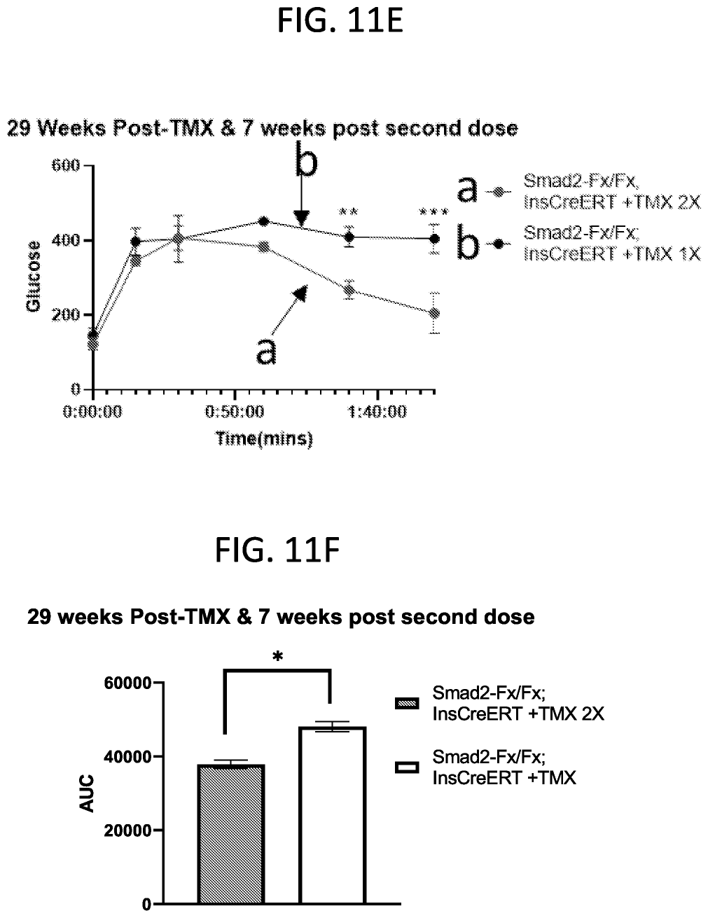

HFD-fed mice had significant weight gain. After 12 weeks of HFD the mice developed impaired glucose tolerance by IPGTT (see FIG. 9). Tamoxifen was given intraperitoneally to glucose-intolerant smad2-βKO mice to induce a beta cell-specific acute smad2-βKO. The acute loss of smad2 expression in the beta cells showed improved glucose tolerance compared to HFD-controls within one week (see FIGS. 10A-10F) and showed continued improvement in glucose tolerance up to nine weeks while the mice are still on HFD (see FIGS. 10C-10F). Beyond nine weeks, a loss of glucose tolerance was detected (see FIG. 11A), possibly due to the development of new beta cells that are smad2-positive. To test whether those new beta cells could be targeted through repeat tamoxifen injection, a second regimen of tamoxifen treatment was given at 22 weeks. Again, within one week after the second tamoxifen treatment, improved glucose tolerance was seen in the mice (see FIGS. 11C, 11D). Continued monitoring for up to 29 (see FIGS. 11E, 11F) and 31 weeks showed the acute smad2-βKO mice retained improved glucose tolerance on HFD.

### Example 8

### Experimental Procedures for Examples 1-7

Mouse Manipulations: Floxed smad2 knock-in (smad2^{fx/fx}) and insulin1^{cre} mice in a pure C57BL/6J background were obtained from the Jackson Laboratory (Bar Harbor, ME). Ins1^{cre} mice were crossed with smad2^{fx/fx} to generate β-cell-specific smad2 knockout mice (smad2-βKO). Equal numbers of males and females were used in each group, aged from 12 to 14 weeks old. For the high fat diet (HFD) experiments, 6-week-old mice were placed on regular chow or HFD (60% kcal from fat, D12492; Research Diets, New Brunswick, NJ) for 12 weeks. All animals were housed under specific pathogen-free conditions.

### Intraperitoneal Glucose Tolerance Test (IPGTT), Glucose Stimulated Insulin Secretion (GSIS), Intraperitoneal Insulin Tolerance Test (IPITT) and Homeostatic Model Assessment-Estimated Insulin Resistance (HOMA-IR):

IPGTT: After overnight fast, mice were injected with 2 g/kg glucose (Sigma-Aldrich, St. Louis, MO). Blood glucose levels were measured at 0, 15, 30, 60, 90, and 120 min after the glucose injection using a glucometer (Contour next EZ).

GSIS: Serum insulin concentrations were measured during IPGTT. Approximately 50 μl blood were collected from the tail vein at 0, 15 and 30 minutes via Microvette CB300Z, clotting activator/serum tubes (Sarstedt, Germany). Collected blood was centrifuged then serum was collected for insulin measurement using mouse ELISA kit (80-INSMSU-E01, Alpco, Salem, NH).

IPITT: After 6 hours fasting mice were injected with (0.075 U/kg) insulin and blood glucose levels were measures at 0, 15, 30, 60 and 90 min after insulin injection.

HOMA-IR: index was calculated using the IPGTT and the GSIS fasting values according to the following formula: HOMA-IR=[fasting glucose (mmol/L)×fasting insulin (mU/L)]÷22.5 (15).

Measurement of Glycated Hemoglobin (HbA1c): Approximately 10 μl of blood was collected from the tail vein in conscious mice and used to measure HbA1c. Mouse HbA1c was determined using DCA Vantage analyzer (Siemens, Tarrytown, NY). This system automatically measures both HbA1c and total hemoglobin, the percent HbA1c in the sample is then calculated as follows: % HbA1c=([HbA1c]/[Total Hemoglobin])×100, and the ratio reported as percent HbA1c (16, 17).

Pancreas Digestion and Islet Isolation: For ex vivo analysis, we isolated the control and smad2-βKO islets using a previously published protocol (Carter et al., Biol Proced Online. 2009; 11:3-31). Briefly, the pancreatic duct was perfused, and the pancreas subsequently digested with Type V Collagenase (1.4 mg/mL). Islets were separated from the exocrine tissue with Histopaque 1100 gradient solution (100 ml Histopaque 1077 and 120 ml Histopaque 1119) (Sigma-Aldrich, St. Louis, MO) and then washed with Hanks' Balanced Salt Solution (Gibco, Grand Island, NY) containing 20 mM HEPES buffer (Gibco, Grand Island, NY) containing 0.2% bovine serum albumin (Sigma-Aldrich, St. Louis, MO). Islets were then handpicked to eliminate any contamination from exocrine tissue.

Islet Perifusion Assay: Isolated islets were left to recover overnight at 37° C. (5% CO₂) in RPMI 1640 medium (Gibco, Grand Island, NY) containing 10% FBS. One-day post harvesting, groups of 50 islets are first placed in a dynamic perifusion system (Amersham Biosciences AKTA FPLC System). The perifusion performed with KREB buffer at basal 2.8 mM glucose at a flow rate of 1 ml/min for 15 min to establish stable basal insulin secretion. Next, islets were perifused with 2.8 mM glucose for 10 min, and fractions of (500 μl) collected every 30 sec. Then, the glucose concentration was increased to 20 mM and fractions of (500 μl) collected every 30 sec for 20 min. Lastly, islets were perfused with 30 mM KCl and fractions of (500 μl) collected every 30 sec for 10 min. After the perifusion, the islets were recollected from the column for genomic DNA measurement. Insulin in the effluent was measured by mouse ELISA kit (80-INSMSU-E0, Alpco, Salem, NH). The fractional insulin secretion rate was calculated as secreted insulin per minute normalized to DNA content.

Ex vivo Glucose Stimulated Insulin Secretion (Static GSIS): Isolated islets (groups of 30 islets) were left to recover overnight at 37° C. (5% CO₂) in RPMI 1640 medium (Gibco, Grand Island, NY) containing 10% FBS. The next day islets were incubated in 2.8 mM glucose for 30 min at 37° C. to establish stable basal insulin secretion, then washed by KREB buffer two times. Islets were then transferred into a new well containing 2 ml of 2.8 mM glucose and 100 μl media collected for time-point 0 insulin measurement. Islets were then incubated in low glucose for 30 min at 37° C. and 100 μl media collected for time-point 1, then transferred into a new well containing 2 ml of 20 mM glucose solution for 30 min at 37° C. and 100 μl media collected for time-point 2. Islets were then recovered for genomic DNA measurement. Insulin levels in the collected media were measured by mouse ELISA kit (80-INSMSU-E0, Alpco, Salem, NH) and normalized to DNA content.

Insulin Content in the Isolated Islets: Ten equal-sized islets per mouse were incubated in 30 μl acid/ethanol (75% ethanol, 0.15M HCl) at 4° C. overnight with gentle rotation to extract insulin, then centrifuged at 14,000 rpm for 10 min. The supernatant was diluted 1:50 and insulin content was measured by mouse ELISA kit (80-INSMSU-E0, Alpco, Salem, NH) and normalized to total islet DNA content.

Calcium Imaging: Isolated islets were dispersed into single cells and plated on glass-bottom culture dishes (MatTek, Ashland, MA). The cells were loaded with 1 μM Fluo-4 (Thermo Fisher Scientific, Waltham, MA) at 37° C. for 30 min then washed with KREB buffer. The cells then were incubated in 2.8 mM glucose for 1 min then exposed to 20 mM glucose for 2 min and lastly to 20 mM KCl for 1 min. The intensity of Fluo-4 at 488 nm was monitored by live imaging of a 20× objective lens of a Zeiss LSM710 confocal microscope and data was analyzed by Zeiss ZEN software.

Imaging was done for on an average of 5 cells per field, three mice were used/group. Isolation of RNA and Quantitative RT-PCR: Total RNA was extracted from isolated islets using RNeasy® Plus extraction kit (Qiagen, Hilden, Germany). RNA was reverse transcribed into cDNA using iScript™ reverse transcriptase (Bio-Rad, Hercules, CA). Quantitative gene expression was analyzed by using TaqMan® Gene expression assays (Thermo Fisher Scientific, MA). Gene expression was normalized using the ΔΔCₜ method, where the amount of target, normalized to an endogenous reference and relative to a calibrator, is given by 2^{-ΔΔCt}, where Cₜ is the cycle number of the detection threshold.

Protein extraction: For protein extraction, liver, skeletal muscle (quadriceps muscle), adipose tissue (gonadal fat pad) samples were placed in approximately 600 μl of NP40 Cell Lysis Buffer (FNN0021) with 1 mM PMSF (36978) and protease inhibitor cocktail (1862209) (Thermo Fisher Scientific). After homogenization on ice, the tissue lysates were incubated on ice for 30 min. After centrifugation at 10,000 g for 30 min at 4° C., the supernatants were collected. Protein concentration was determined by bicinchoninic acid (BCA) protein assay QuantiPro™ BCA Assay Kit (QPBCA, Millipore-Sigma).

Western Blot: Lysates from isolated islets, liver, skeletal muscle and adipose tissue were separated on SDS-PAGE gels and subsequently transferred to PVDF membranes (Millipore). Membranes were blocked in 5% milk in TBST for 1 h then incubated with rabbit monoclonal antibodies (Cell Signaling Technology) against total Smad-2/3 (8685S), Akt (4691S), Phospho-Akt (4060S), Phospho-PERK (3179S), Phospho-eIF2α (3398S), and β-actin (4970S) overnight at 4° C. Membranes were washed and incubated with horseradish peroxidase-conjugated anti-rabbit 10 (1705046, Bio-Rad) for 1 h. Bands were detected using Luminescent Image Analyzer LAS-3000™. The density of each band was quantified using ImageJ software.

Immunohistochemistry: All pancreas samples were fixed with 4% paraformaldehyde (PFA) in PBS for 48 h, dehydrated using 30% sucrose overnight, then embedded in optimal cutting temperature (OCT) compound, snap-frozen by liquid nitrogen, and sectioned at 7 μM. For IHC, antigen retrieval was performed (heat and/or acid buffer). Slides were incubated with primary antibodies at 4° C. overnight and were then incubated with Fluorescent conjugated (FITC, CY3) secondary antibodies (Jackson ImmunoResearch Labs, West Grove, PA) for 1 h at RT the following day. Nuclear staining and mounting were performed using Fluoroshield™ with DAPI (Sigma-Aldrich, St. Louis, MO).

Primary antibodies for immunostaining were guinea pig polyclonal anti-Ins (ab195956), rabbit polyclonal anti-Pdx1 (ab47267), rabbit monoclonal anti-Nkx6.1 (ab221544), mouse monoclonal anti-NeuroD1 (ab60704), rat monoclonal anti-BrdU (ab152095) purchased from Abcam; rabbit monoclonal anti-MafA (79737S) (Cell Signaling Technology, MA); and rabbit polyclonal anti-phospho-Smad-2 (Ser465/467) (Thermo Fisher Scientific, MA).

Oil-Red-O staining: PFA-fixed liver samples were dehydrated in 30% sucrose, and snap-frozen with liquid nitrogen after embedding in OCT. Frozen sections (7 μM) were used for Oil-red-O staining to monitor steatosis (Singh et al., Cell. 2018; 175(3):679-94 e22.). Briefly, sections were kept in PBS for 10 min. and washed with 60% isopropanol. Liver sections were placed in Oil-red-O (00625, Sigma-Aldrich, St. Louis, MO) solution (0.5% in 60% isopropanol) for 30 min. then rinsed with 60% isopropanol to remove the non-specific staining and counterstained with Mayer's hematoxylin (Sigma-Aldrich, St. Louis, MO).

Quantifications and Data Analysis: For quantification of β-cell proliferation, the percentage of BrdU+ per insulin+ cells were calculated, BrdU+ cells were manually quantified from at least six sections that were 100 uM apart for each pancreas. Pdx1+, MafA+, and NKX6.1+ β-cells were manually quantified from at least six sections that were 100 μM apart for each pancreas. The β-cell mass/area was quantified as previously described (El Gohary et al., Diabetes. 2014; 63(1):224-36). In brief, ten sections at 100 μM intervals from the whole pancreas were immunostained for insulin and DAPI and imaged using a confocal microscope. Captured images of entire sections were analyzed using ImageJ software. Average β-cell mass was calculated by multiplying islet (insulin-positive): pancreas area ratio with pancreatic weight.

All the data were analyzed using GraphPad Prism (GraphPad Software, San Diego, CA 92108). All values were depicted as means±SEM. The differences between the two groups in the IPGTT, perfusion study and Ca²¹ tracing study were analyzed by using area under the curve (AUC). AUC was calculated using the trapezoid method. All data were statistically analyzed by unpaired Student t-test and statistically significant differences are shown for p<0.05(*), p<0.01(**), and p<0.001(***).

### Example 9

### Additional Results

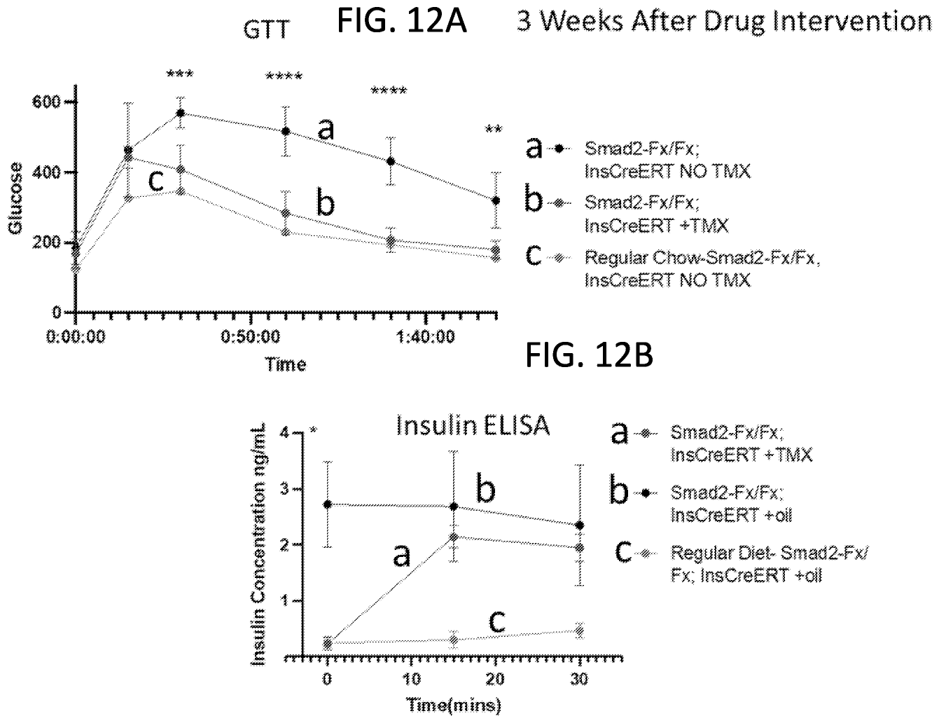

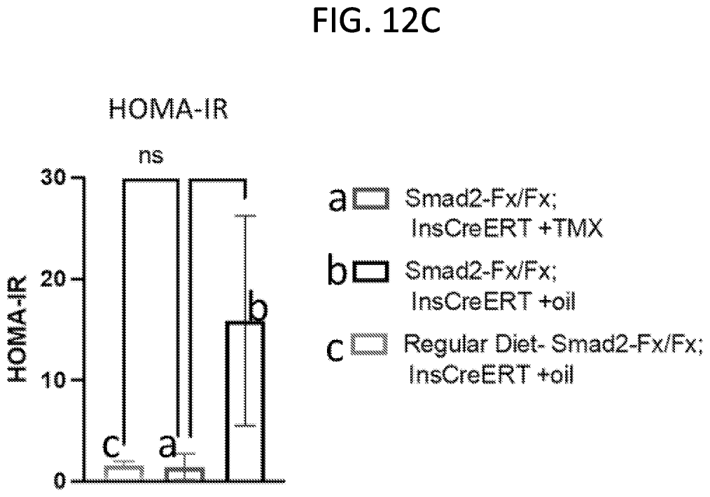

FIGS. 12A-12C show that within three weeks after deleting smad2 expression beta cells show improved glucose homeostasis. Starting at six weeks of age, Ins-cre-ERT; Smad2 fx/fx mice were fed high fat diet (60% kcal from fat, D12492; Research Diets) for 12 weeks. After twelve weeks, 75 mg tamoxifen/kg body weight dissolved in corn oil (10 mg/ml) was given intraperitoneally for five days. For the IPGTT, sixteen-hour-fasted mice were intraperitoneally injected with 2 g/kg glucose (Sigma-Aldrich). Blood glucose levels were measured at 0, 15, 30, 60, 90, and 120 min after the glucose injection using a glucometer (Contour next EZ). Insulin level from samples collected at 0, 15 and 30 minutes during IPGTT was measured using a mouse ELISA kit (ALPCO) and normalized to the DNA content. The HOMA-IR index was calculated using the following formula: HOMA-IR=[fasting glucose (mmol/1)×fasting insulin (mU/1)]+22.5. The mice are described above in Example 8.

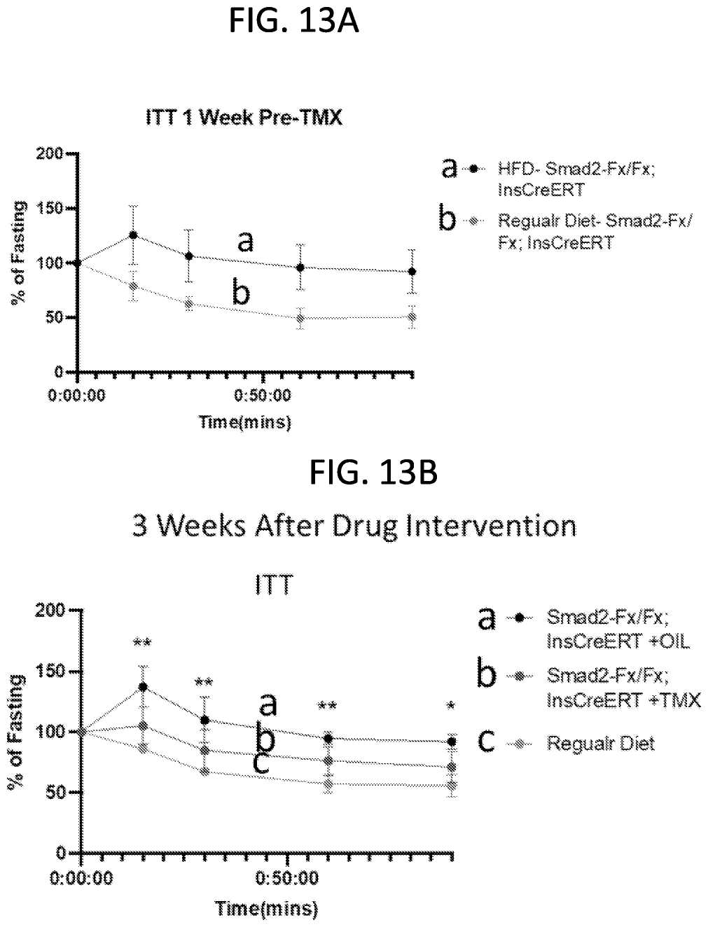

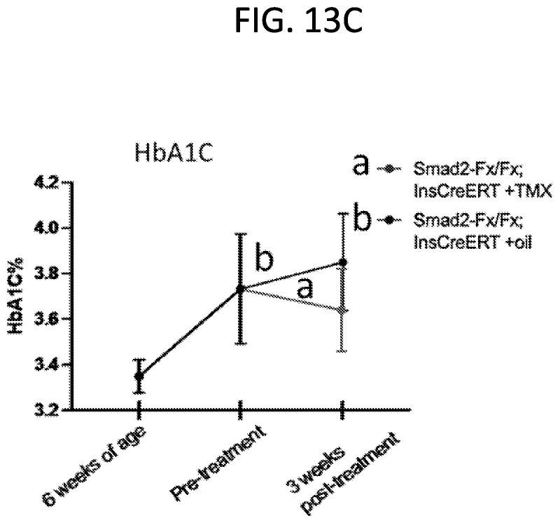

FIGS. 13A-13C show that, within three weeks after deleting smad2 expression, peripheral insulin sensitivity improved in the knockout mice. Starting 6 weeks of age, Ins-cre-ERT; Smad2 fx/fx mice were fed high fat diet (60% kcal from fat, D12492; Research Diets) for 12 weeks. After twelve weeks, 75 mg tamoxifen/kg body weight dissolved in corn oil (10 mg/ml) was given intraperitoneally for five days. For the IPGTT, sixteen-hour-fasted mice were intraperitoneally injected with 2 g/kg glucose (Sigma-Aldrich). Blood glucose levels were measured at 0, 15, 30, 60, 90, and 120 min after the glucose injection using a glucometer (Contour next EZ). For the insultin tolerance text (ITT), six-hour-fasted mice were injected i.p. with 0.75 units/kg regular insulin. Blood glucose was measured from the tail vein at 0, 15, 30, 60, and 90 minutes after insulin injection using the glucometer. Approximately 10 μl of blood was collected from the tail vein in conscious mice to measure HbA1c using the DCA Vantage Analyzer (Siemens). This system automatically measures both HbA1c and total hemoglobin, and the percent HbA1c is then calculated as follows: % HbA1c=([HbA1c]/[Total Hemoglobin])×100. The ratio is reported as the percent HbA1c. The mice are described above in Example 8.

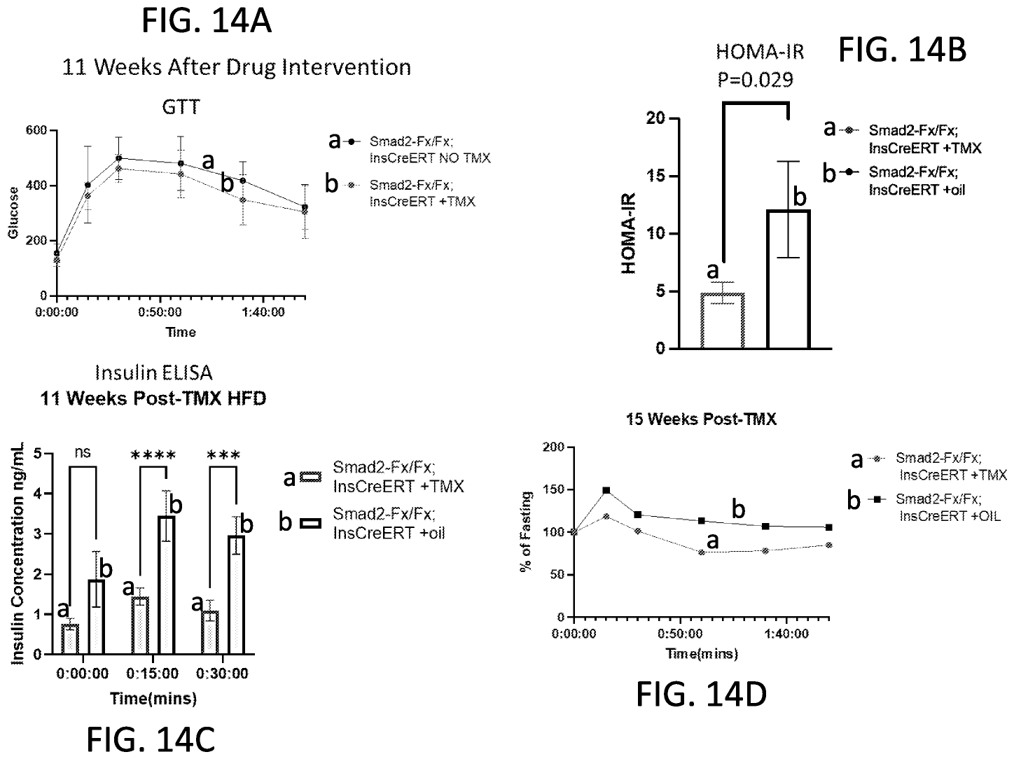

FIGS. 14A-14D show that the loss of smad2 expression maintains improved beta cell function, and insulin sensitivity for more than 11 weeks. Starting 6 weeks of age, Ins-cre-ERT; Smad2 fx/fx mice were fed high fat diet (60% kcal from fat, D12492; Research Diets) for 12 weeks. After twelve weeks, 75 mg tamoxifen/kg body weight dissolved in corn oil (10 mg/ml) was given intraperitoneally for five days. For the IPGTT, sixteen-hour-fasted mice were intraperitoneally injected with 2 g/kg glucose (Sigma-Aldrich). Blood glucose levels were measured at 0, 15, 30, 60, 90, and 120 min after the glucose injection using a glucometer (Contour next EZ). For in vivo GSIS, insulin level from samples collected at 0, 15 and 30 minutes during IPGTT was measured using a mouse ELISA kit (ALPCO) and normalized to the DNA content. For the ITT, six-hour-fasted mice were injected i.p. with 0.75 units/kg regular insulin. Blood glucose was measured from the tail vein at 0, 15, 30, 60, and 90 minutes after insulin injection using the glucometer. The mice are described above in Example 8.

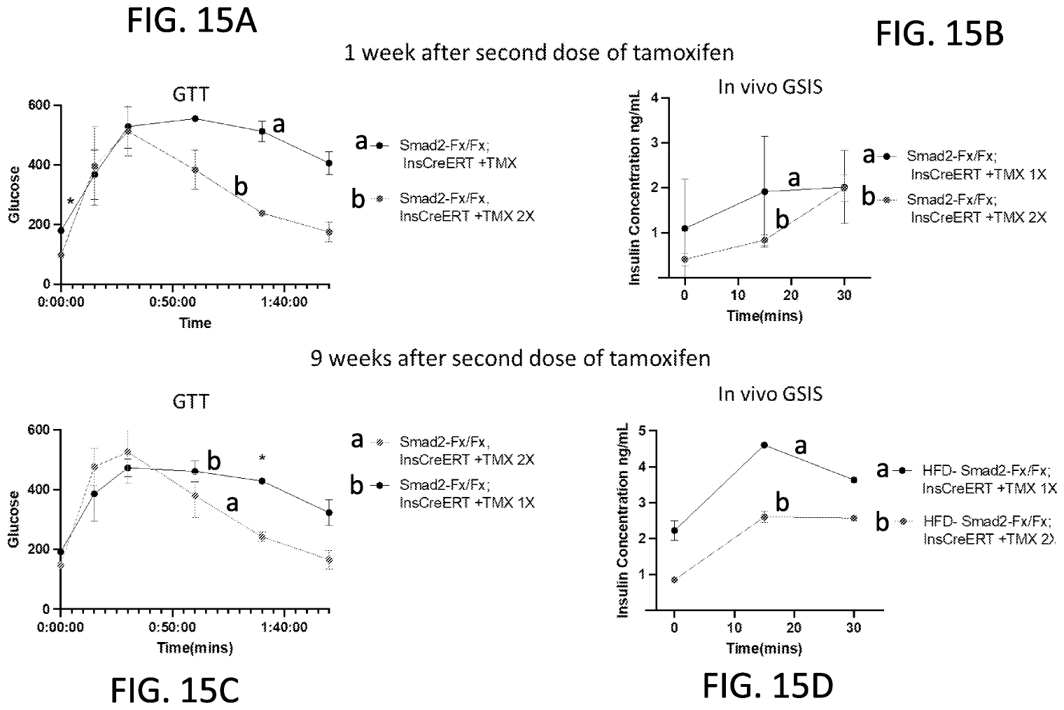

FIGS. 15A-15D show that a repeat intervention restores glucose homeostasis in smad2 knockout mice maintained on a high fat diet (HFD). After nineteen weeks of first tamoxifen regimen, Ins-cre-ERT; Smad2 fx/fx mice maintained on fed high fat diet failed a glucose tolerance test. The tamoxifen treatment was repeated at 75 mg tamoxifen/kg body weight, intraperitoneally for five days. Then an IPGTT was done one week and nine weeks after the second dose. Using the protocol described above, sixteen-hour-fasted mice were intraperitoneally injected with 2 g/kg glucose (Sigma-Aldrich). Blood glucose levels were measured at 0, 15, 30, 60, 90, and 120 min after the glucose injection using a glucometer (Contour next EZ). For in vivo GSIS, insulin level from samples collected at 0, 15 and 30 minutes during IPGTT was measured using a mouse ELISA kit (ALPCO) and normalized to the DNA content. The mice are described above in Example 8.

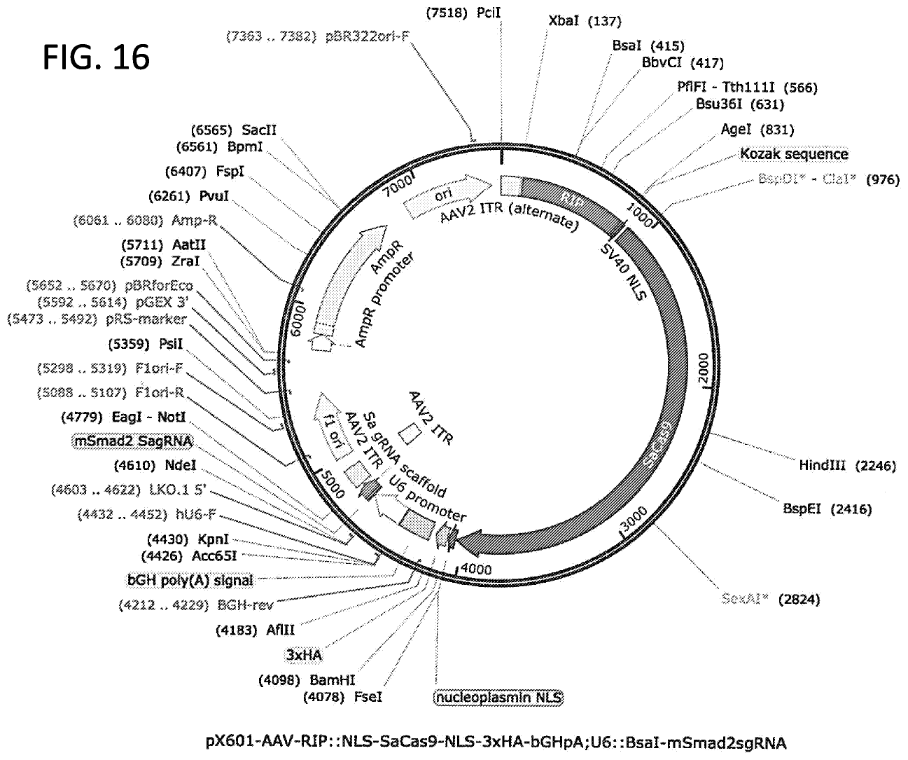

FIGS. 16A-16D show a CRISPR-Cas9 strategy to inhibit smad2 expression in cell lines. The following sequences were used.

Smad2 [SagRNA#1]
(SEQ ID NO: 24)
TCTTCAGGTTTCACACCGGA
Smad2 [SagRNA#2]
(SEQ ID NO: 25)
CTAACCCGAATGTGCACCAT
Smad2 [SagRNA#3].
(SEQ ID NO: 26)
GCATACTATGAACTAAACCA

Sa gRNA is made up of two parts: crispr RNA (crRNA), a 17-20 nucleotide sequence complementary to the target DNA, and a tracr RNA, which serves as a binding scaffold for the saCas nuclease. The crRNA targeted smad2 gene sequences are shown as SEQ ID NOs: 24-26.

Scramble [SagRNA#4]
(SEQ ID NO: 27)
GTGTAGTTCGACCATTCGTG

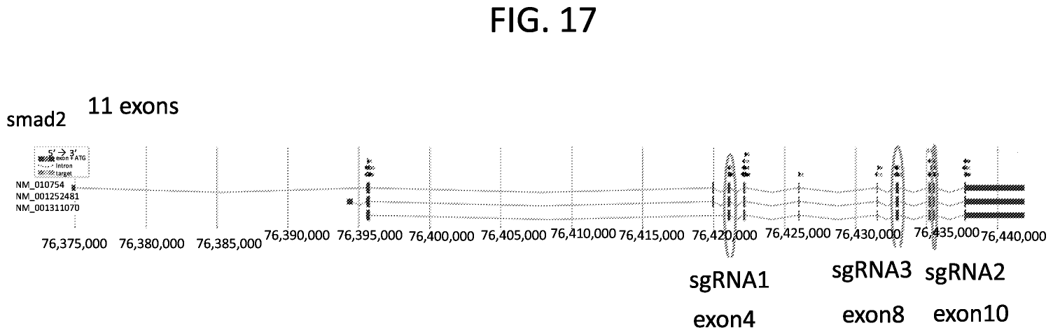

The backbone plasmid is from Addgene: #61591, and the CMV promoter was replaced with the rat insulin promoter (RIP), see FIG. 16. A CRISPR-Cas9 strategy targeting exons 4, 8 and 10 is shown in FIG. 17 using these three sgRNA constructs.

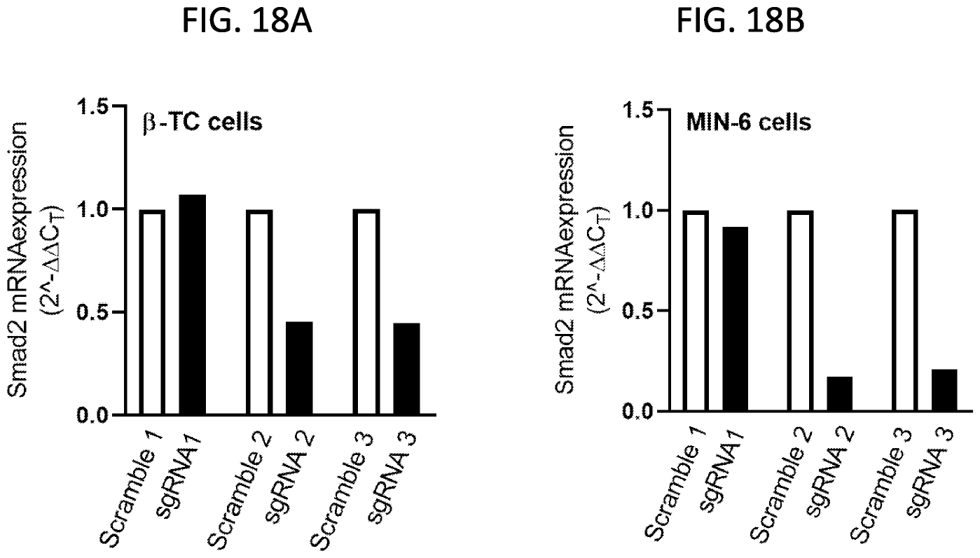

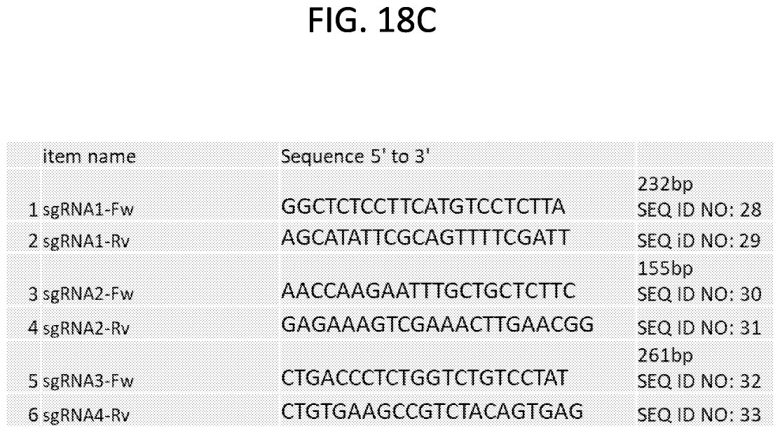

FIGS. 18A-18C show results from Crispr-Cas9 mediated knockdown of smad2 in β-TC and Min-6 cells. The MIN-6 and β-TC cell lines were purchased from the American Type Culture Collection (Manassas, VA). Each cell line was cultured in Dulbecco's modified essential medium supplemented GLUTAMAX™ Supplement, pyruvate with 10-15% fetal bovine serum and antibiotics. MIN-6 and β-TC were plated for ˜50-60% confluency in 6 well plates for 24-h. Cultured cells were infected with CRISPR-Cas9 sgRNA1-3 and CRISPR-Cas9 scramble sgRNA with 50000 multiplicity of infection (MOI) for 3 days. Genomic DNA was extracted from virus infected cultured MIN-6 and β-TC cells using PURELINK™ Viral RNA/DNA Mini Kit (Invitrogen, Carlsbad, CA). Extracted genomic DNA were amplified using the primers shown in FIG. 18C (SEQ ID NOs: 28-33). The in vitro treatment of MIN6 cells and Beta-TC cells show that the contruct and the knockout strategy was effective, and can be adapted for in vivo use.

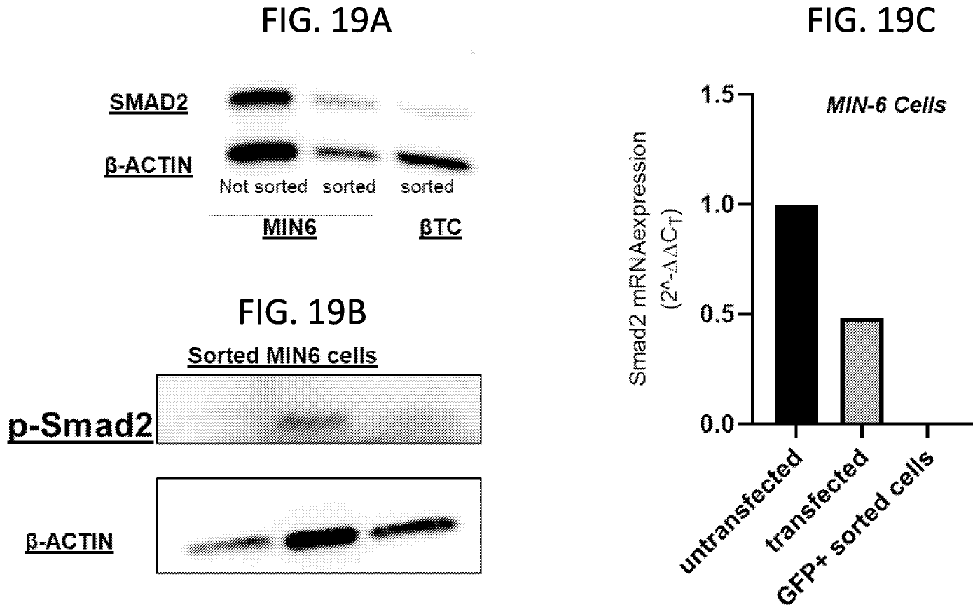

FIGS. 19A-19C show results from experiments where Min6 cells or beta-TC cells were treated with AAV6-GFP-U6-mSMAD2_shRNA for three days. A lysate was prepared from total or sorted cells. RT-PCR, and Western blot for Smad2, phospho Smad2 and Beta-actin was performed. Min6 and beta-TC cells sorted after transfection show near complete ablation of smad2 expression (FIG. 19A). Phospho smad2 expression is nearly absent in Min6 cells after AAV6-GFP-U6-mSMAD2_shRNA treatment (FIG. 19B). RT-PCR quantification of MIN6 mRNA after treated with AAV6-GFP-U6-mSMAD2_shRNA show complete absence of mRNA in sorted cells (FIG. 19C).

The following construct was used:

AAV6-GFP-U6-m-SMAD2-shRNA sequence:
(SEQ ID NO: 34)
5′-CCGGTGGTGTTCAATCGCATACTAT-CTCGAG-
ATAGTATGCGATTGAACACCA-TTTTTG-3′
and
a targeting sequence of
(SEQ ID NO: 35)
TGGTGTTCAATCGCATACTAT.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method of treating a subject with type 2 diabetes, comprising: administering to the subject a therapeutically effective amount of an inhibitor that decreases expression of Mothers Against Decapentaplegic Homolog (smad) 2, thereby treating the type 2 diabetes in the subject.

2. The method of claim 1, wherein the inhibitor is an inhibitory nucleic acid molecule.

3. The method of claim 2, wherein the inhibitory nucleic acid molecule is a small interfering RNA (siRNA) or a short hairpin RNA (shRNA).

4. The method of claim 2, comprising administering to the subject a viral vector transcribed to form the inhibitory nucleic acid molecule.

5. The method of claim 4, wherein the viral vector is a lentivirus vector, an adenovirus vector or an adeno-associated virus (AAV) vector.

6. The method of claim 5, wherein the viral vector is the AAV vector.

7. The method of claim 5, wherein the viral vector transcribed to form the inhibitory nucleic acid molecule further comprises a beta cell-specific promoter operably linked to a nucleic acid molecule encoding the inhibitory nucleic acid molecule, and
wherein the viral vector is administered to a pancreatic duct of the subject.

8. The method of claim 7, wherein the promoter is an insulin promoter.

9. The method of claim 2, wherein the inhibitory nucleic acid is a ribozyme.

10. The method of claim 2, wherein the inhibitory nucleic acid is a morpholino oligonucleotide.

11. The method of claim 1, wherein the inhibitor comprises a gRNA and a vector encoding a Cas9 nuclease.

12. The method of claim 11, comprising administering to a pancreas of the subject:
a) a beta cell-specific promoter operably linked to a nucleotide sequence encoding the Cas9 nuclease, and
b) a U6 promoter operably linked to one or more nucleotide sequences transcribed to form the gRNA, wherein the gRNA hybridizes with a smad2 gene,
wherein components (a) and (b) are located on a same or different expression vector, and
the expression vector is expressed in transformed pancreatic endocrine cells, whereby the gRNA targets the smad2 gene for cleavage by the Cas9 nuclease, thereby decreasing expression of smad2 in the pancreatic endocrine cells.

13. The method of claim 1, wherein the inhibitor or vector is administered intraductally into a pancreatic duct.

14. The method of claim 13, wherein administering intraductally comprises the use of endoscopic retrograde cholangiopancreatography (ERCP).

15. The method claim 1, comprising administering an additional agent to the subject.

16. The method of claim 15, wherein the agent is metformin.

17. The method of claim 1, wherein administering the therapeutically effective amount of the inhibitor reduces glucose intolerance in the subject.

18. The method of claim 1, wherein administering the therapeutically effective amount of the inhibitor reduces plasma glucose levels in the subject.

19. The method of claim 1, wherein the subject is human.

20. (canceled)
